(19) United States
(12) Patent Application Publication
(10) Pub. No.: US 2023/0257716 A1
(43) Pub. Date: Aug. 17, 2023

(54) METHODS ENABLING INFECTION AND DIFFERENTIATION OF HUMAN DISTAL LUNG ORGANOIDS BY SARS-COV-2 AND OTHER PATHOGENS

(71) Applicant: The Board of Trustees of the Leland Stanford Junior University, Stanford, CA (US)

(72) Inventors: Calvin Jay Kuo, Palo Alto, CA (US); Shannon Choi, Redwood City, CA (US); Manuel R. Amieva, Stanford, CA (US); Catherine A. Blish, Stanford, CA (US); Maria del Mar Margalef Catala, Redwood City, CA (US); Arjun Rustagi, Redwood City, CA (US)

(21) Appl. No.: 18/005,167

(22) PCT Filed: Jul. 16, 2021
(86) PCT No.: PCT/US2021/042086

§ 371 Date: Jan. 11, 2023

Related U.S. Application Data
(60) Provisional application No. 63/053,079, filed on Jul. 17, 2020.

(51) Int. Cl. C12N 5/071; G01N 33/50

(52) U.S. Cl. CPC C12N 5/0689; G01N 33/5082; C12N 2501/11; C12N 2501/155; C12N 2501/15; C12N 2503/04; C12N 2513/00; C12N 2533/54; C12N 2533/90; G01N 2333/08

(57) ABSTRACT

Abstract: We describe a robust human adult distal lung organoid method with a procedure for everting organoids to essentially turn them inside out. This then relocates the apical ACE2-expressing surfaces of cells to the organoid exterior, where they can then be easily infected by SARS-CoV-2 added to the tissue culture medium. Further, this method can be used for infection of any distal lung pathogen that infects apically. Alternatively, if a pathogen interacts basolaterally then eversion is not necessary, and the human adult distal lung organoids can be infected as is.

### CROSS REFERENCE TO RELATED APPLICATION

The present application claims the benefit of and priority to U.S. Provisional Patent Application No. 63/053,079 filed Jul. 17, 2020, the entire disclosure of which is hereby.

### STATEMENT REGARDING FEDERALLY SPONSORED RESEARCH

This invention was made with Government support under contract U19AI116484 awarded by the National Institutes of Health. The Government has certain rights in the invention.

### BACKGROUND

The distal lung, including terminal bronchioles and alveoli, performs essential gas exchange functions which can be significantly compromised by infectious diseases. For example, SARS-CoV-2 infection can elicit severe distal lung COVID-19 pathology with life-threatening pneumonia and respiratory failure. Further, numerous bacterial and viral pathogens can greatly afflict the distal lung. The limited understanding of COVID-19 pneumonia pathogenesis during a worldwide pandemic has highlighted a pressing need for robust in vitro culture systems allowing study of distal lung pathologies in primary human cells. Unfortunately, the human adult distal lung, either terminal bronchioles or alveoli, has been historically refractory to primary 3D tissue culture or even organoid technologies, with a notable lack of solutions allowing long-term, feeder free, chemically defined passage.

Prior culture of adult human airway progenitors has been limited to basal cells from upper airway, namely trachea or upper bronchi while alveolar type 2 cell cultures are short-lived and require feeder cells that produce undefined signals. iPSCs can be differentiated to lung epithelial lineages using feeder layers but fetal gene expression is often observed. By contrast, in the methods disclosed herein, long-term, feeder-free, chemically defined 3D organoid culture of distal human lung, can be established and are reproducible.

Further, organoid cultures often grow as cystic structures with their apical surfaces directed inwards towards a central lumen, which renders the apical aspects inaccessible to pathogens added externally to the tissue culture medium. This is particularly important for agents such as SARS-CoV-2 that interact with apical receptors such as ACE2. Because of this, SARS-CoV-2 infection of organoids from other tissues such as intestine have resorted to mechanically breaking apart organoids to allow SARS-CoV-2 to interact with the apical internal surface of the organoid.

It has not been previously possible to culture human distal lung (alveoli, terminal bronchioles) long term in a feeder free, chemically defined manner. Human alveolar type 2 (AT2) cell cultures have been reported but are short-lived, and the long-term self-renewal capacity of human AT2 cells thus remains unknown. Furthermore, existing AT2 cell culture protocols achieve only minimal expansion and require feeder cells producing unknown factors, limiting their biological characterization and screening utility. Directed differentiation of induced pluripotent stem cells (iPSCs) to AT2 cells can be limited by efficiency, feeder dependence and persistent fetal gene expression, suggesting immaturity. Here, we established long-term, feeder-free, chemically defined 3D organoid culture of distal human lung, including AT2 and basal stem cells, and applied this method to SARS-CoV-2 modeling.

The lack of long-term human distal lung culture systems has precluded functional testing of the proliferative capacity of putative human distal lung stem cell populations, which are therefore largely inferred from mouse studies.

### SUMMARY

Compositions and methods are provided for culture of distal human lung organoids; modeling viral infection of cells in such organoids; and for screening of candidate agents for treatment of such viral infection. The methods described herein relate to organoid in vitro cultures derived from human tissue of the distal lung, inclusive of alveolar and terminal bronchiolar cells. The in vitro cultured cells provide tools for a novel method for viral infection, and for investigation of the prevention and treatment of such infections.

In some embodiments, provided are feeder-free, chemically-defined culture of distal human lung progenitors as organoids, derived from distal lung tissue. The cultures are optionally clonally derived from single adult human alveolar epithelial type II (AT2) and/or KRT5⁺ basal cells. AT2 cells in the organoids exhibit AT1 transdifferentiation potential. Basal cell organoids can be characterized by progressively developed lumens lined by differentiated club and ciliated cells. These long-term, feeder-free organoid culture of human distal lung alveolar and basal stem cells, coupled with single cell analysis, identifies unsuspected basal cell functional heterogeneity and allows progenitor identification within a slowly proliferating human tissue.

Influenza virus strain H1N1 broadly injures both airway and alveolar epithelium. Both basal and AT2 organoids from human distal lung cultures are avidly infected influenza H1N1. Screening of diverse antiviral compound classes in H1N1-infected organoids has revealed differential effectiveness, demonstrating utility for scalable therapeutics analysis.

In some embodiments, organoids, e.g. basal or alveolar organoids, have everted polarity, in which differentiated club and ciliated cells are relocated from the organoid lumen to the exterior surface, thus displaying the ACE2 receptor on the outwardly-facing apical aspect. Basal and AT2 “apical-out” organoids can be infected with pathogens that utilize the ACE2 receptor, including without limitation SARS-CoV-2. Club cells are identified as a novel target population for SARS-CoV-2 infection.

In some embodiments a method is provided for everting organoids to essentially turn them inside out. This relocates the apical ACE2-expressing surfaces of cells to the organoid exterior, where they can then be easily infected by SARS-CoV-2 added to the tissue culture medium. Further, this method can be used for infection of any distal lung pathogen that infects apically. Alternatively, if a pathogen interacts basolaterally then eversion is not necessary, and the human adult distal lung organoids can be infected as is. A second consequence of the apical-basal polarity organoid eversion in suspension culture is the induction of differentiation. The basal organoids undergo a profound induction of ciliated cell differentiation, while the alveolar type 2 cell organoids undergo strong differentiation to alveolar type 1 cells. Thus, this method also allows facile generation of human ciliated and alveolar type 1 cells in suspension culture. Previously, alveolar type 1 differentiation required culture on glass surfaces, and ciliated differentiation required culture as monolayers in a two-dimensional air liquid interface. Neither of these prior methods are very scalable in contrast to our current suspension culture technique. The apical-basal polarity eversion technique allows the apical aspect of the organoid cells to be brought to the exterior of the organoid by a gentle suspension culture method, allowing apical infection without resorting to mechanical shearing of the organoids.

Using this method it has been demonstrated that SARS-CoV-2 infects of distal lung organoids, either alveoli or terminal bronchioles, and it is demonstrated that club cells in terminal bronchioles are a novel SARS-CoV-2 target cell.

Drug screening can be performed using human alveoli or distal bronchiolar tissue for SARS-CoV-2 or other pulmonary infectious pathogens, e.g. bacteria, viruses, and the like. In some aspects, a method is provided for in vitro screening for agents for their effect on cells of different tissues, including processes of viral infection initiation and treatment, and including the use of experimentally modified cultures described above. Tissue explants cultured by the methods described herein are exposed to candidate agents. Agents of interest include pharmaceutical agents, e.g. small molecules, antibodies, peptides, etc., and genetic agents, e.g. antisense, RNAi, expressible coding sequences, and the like, e.g. expressible coding sequences for candidate secreted growth factors, cytokines, receptors or inhibitors thereof, or other proteins of interest, and the like. In some embodiments the effect of candidate therapeutic agents on viral infection-related immune responses or their downstream effects is determined, for example where agents may include, without limitation, chemotherapy, monoclonal antibodies or other protein-based agents, radiation/radiation sensitizers, cDNA, siRNA, shRNA, small molecules, and the like. Methods are also provided for using the organoid culture to screen for agents that modulate tissue function.

### BRIEF DESCRIPTION OF THE DRAWINGS

FIG. 1. Clonogenic expansion of human distal lung organoids in chemically defined conditions. a. Day 14 organoid culture of dissociated unfractionated human distal lung, H&E. Cystic and solid organoids are denoted. Scale bar=100 μm. b-c, Time lapse transmission confocal images of solid (b) and cystic (c) organoids originating from single cells, scale bar=100 μm. d, Combinatorial whole-mount immunofluorescence (IF) of organoid cultures for anti-KRT5 (basal) SCGB1A1 (club) and SFTPC (AT2), scale bar=100 μm, day 32. e-g, Analysis of alveolar organoids, day 32. e, H&E image of cystic AT2 organoid, scale bar=25 μm. f, Combinatorial whole-mount fluorescence for anti-SFTPC, anti HTII-28, phalloidin and DAPI, scale bar=50 μm. g, anti-Ki67 and DAPI fluorescence of adjacent section of (e) h-j, Analysis of basal organoids, day 32. h, H&E of basal organoid, scale bar=50 μm. i, Combinatorial whole-mount fluorescence for anti-KRT5 and DAPI, scale bar=100 μm, j, Ki67 immunostaining of (i). k, Purification schema to isolate epithelial cells from distal human lung involving negative MACS bead depletion of CD45³⁰ hematopoietic cells, endothelial cells and fibroblasts, followed by positive FACS selection for EPCAM⁺ epithelium. I, Representative FACS from the purification of (k) demonstrating >99.9% EPCAM⁺ purity (orange) upon re-analysis versus unstained controls (grey). m, Proliferation of EPCAM⁺ cells purified from distal lung as in (k) after day 10 of organoid culture with specified growth factors N=Noggin, E=EGF, W=WNT3A, R=RSPO1 from 3 technical replicates, error bars=SEM, *=p<0.05 n-p, Clonality mixing studies. n, Schema of mixing studies of lentivirus-GFP-and lentivirus-mCherry-expressing cells to determine clonality. o, Representative live fluorescent imaging of resultant green and red organoids from (m), scale bar =500 pm. p, Quantitation of red, green, or chimeric, distal lung organoid cultures from two separate lung donors (1, 2) after initial and serial passaging (P1=passage 1). q-v, scRNA-seq of day 28 total distal lung organoid cultures. q, Unsupervised clustering of scRNA-seq of day 28 total distal lung organoid cultures demonstrates AT2, basal, and club cell populations, with canonical markers of these cell types (SFTPC (AT2), KRT6A/KRT5 (Basal), SCGB1A1 (club)). r, t-SNE plot of 7,285 individual cells from (q) displays the cell classes. s, Violin plots of (r). t-v, Feature plots highlight distribution and log₁₀ UMI counts corresponding to q-s.* p<0.05, ** p<0.01, two-tailed Student's t-test; ns, non-significant. For scRNA-seq analysis in q-v, a modified Kruskal-Wallis Rank Sum Test was performed to determine significance of differential marker gene expression for AT2, basal, and club, with all p-values<0.001.

FIG. 2. Long-term clonogenic culture of human basal and AT2 cells. a, Schema of FACS isolation of AT2 cells from human mixed distal lung organoids as EPCAM⁺LysoTracker⁺ AT2 cells followed by long-term clonogenic culture. b, AT2 organoid culture from (a), Brightfield day 180, scale bar=200 μm. c, H&E from (b), day 180, scale bar=50 μm. d, Transmission electron microscopy image of representative AT2 organoid from b-c, day 32, LB=lamellar body, scale bar=5 μm. e, Combinatorial IF staining for AT1 (HTI-56) and AT2 (HTII-280) cell markers in AT2 organoids as in (a), day 32, scale bar=50 μm. f, AT2 organoids from (e) dissociated into single cells and cultured on glass with DMEM/F12 and 5% fetal calf serum for 10 days, scale bar=50 μm. g, AT2 organoid proliferation with differing combinations of niche factors and PORCUPINE inhibitor C59 (1 μM) from 3 technical replicates, error bars=SEM, *=p<0.05 h, Representative image of clonal mixing studies from stroma-depleted, EPCAM⁺Lysotracker⁺ and lentivirally marked AT2 cells demonstrating presence of mCherry⁺ or GFP⁺ but not chimeric organoids carried out as in (FIG. 1n), passage 1 after lentiviral infection, scale bar=200 μm. i, scRNA-seq analysis of clonal AT2 organoids from EPCAM⁺Lysotracker⁺ cells highlighting homogeneous distribution of AT2 SFTPA1/B/C as well as mitotic (PCNA, CDK1) mRNAs but absence of AT1 (PDPN), basal (KRT5) and club (SCGB1A1) mRNAs among 2,780 single cells from enriched AT2 organoids. logo UMI counts, day 89 cumulative culture. j-k, Spontaneous lumen formation and differentiation of basal organoids in day 26 mixed distal lung culture. A spectrum of formation of interior lumens lined by acetylated tubulin⁺(AcTUB) ciliated and SCGB1A1⁺ club cells, along with the SARS-CoV-2 receptor ACE2 is observed, scale bar=20 μm. I-o, Clonogenic culture of human distal airway basal organoids. I, Spontaneous club cell differentiation within basal culture, day 38, scale bar=50 μm. m, Ciliated organoid confocal transmission image from Supplementary Video 1, scale bar=20 μm. n, H&E and immunostaining of ciliated and club cell markers of 2D air liquid interface cultures initiated from d14 Ficoll-sedimented basal cell organoids, scale bar=50 μm. o, Representative image of clonal mixing studies from stroma-depleted, Ficoll-purified and lentivirally-marked basal organoid cells demonstrating mCherry⁺ or GFP⁺ but not chimeric organoids as in FIG. 1n, passage 1 after lentiviral infection, scale bar=200 μm.

FIG. 3. scRNA-seq-based discovery and functional validation of a proliferative human TNFRSF12A^{hi} basal cell subset in organoids. a-c, scRNA-seq analysis and subclustering of KRT5⁺ basal cells from distal lung organoid culture day 28. a, Basal cells from FIG. 1r are subclustered into Basal 1 (orange), characterized by proliferation and developmental programming and Basal 2 (blue), enriched for structural, cytoskeletal and calcium binding protein gene expression, t-SNE. b, Heat map of (a). c, Feature plots of (b) highlight selective enrichment of basal marker transcripts in Basal 1 versus Basal 2. logic, UMI counts are indicated. d, Left, Violin plot of scRNA-seq analysis from FIG. 4a depicting KRT5 expression among EPCAM⁺ITGA6⁺ITGB4⁺ single cells (purple, i.e. tandem expression of all three genes) versus the remainder of cells (gray), p<0.001 Kruskal-Wallis Rank Sum Test. Middle, t-SNE visualization of TNFRSF12A and ITGA6 expression from the left panel among cells with EPCAM⁺ITGA6⁺ITGB4⁺ gene expression and subdivision by high (top quartile, orange), medium (pink) and low (bottom quartile, navy blue) mRNA expression. Right, Proliferation-associated gene expression is progressively enriched for scRNA-seq cell fractions of in EPCAM⁺ITGA6⁺ITGB4⁺ cells that are stratified for low, medium, or high expression of TNFRSF12A mRNA but for similar gradations of ITGA6 mRNA, n.s.=not significant, ***=p<0.001 Chi-square test. e-g, Prospective isolation and clonogenicity of TNFRSF12A^{hi} cells from mixed distal lung organoids. e, FACS gating strategy to subfractionate Basal 1 into TNFRSF12A^{hi} versus TNFRSF12A^{neg} from (EPCAM⁺ITGA6⁺ITGB4⁺) Basal 1 cells. Populations shown were pre-gated on live singlets. f, Representative brightfield image of TNFRSF12A^{hi} versus TNFRSF1 2Aneg fractions from (f) after 14 days of organoid culture. g, Quantitation of organoid formation in (f), each data point represents the mean of technical replicates of an organoid culture from a unique individual, ***=p<0.001 two-tailed Student's t-test.

FIG. 4. In vivo localization, prospective isolation, and clonogenic activity of the proliferative TNFRSF12A^{hi} subset of Basal 1 from intact human lung. a, Left, Representative multicolor immunofluorescence of freshly fixed human distal lung with overlap of monoclonal anti-KRT5 (red) and polyclonal anti-TNFRSF12A (green) in small airways, DAPI=blue. Middle, enlargement of the yellow boxed area from the left panel. Right, representation of the middle panel without DAPI channel, scale bar=100 μm. b, Immunofluorescence of KRT5 (red) and TNFRSF12A (green) freshly fixed human distal lung small airways from an additional individual, scale bar=100 μm. c, Representative immunofluorescence of TNFRSF12A (green), KRT5 (red), and p63 (blue) demonstrating TNFRSF12A overlap in a subset of KRT5⁺p63⁺ cells. d, Proliferation of TNFRSF12A⁺ airway basal cells in distal lung histologic sections. IF for KRT5 (red), TNFRSF12A (green) and K167 (white) with DAPI (blue). scale bar=100 μm. e, Mitotic index calculation of TNFRSF12A⁺ KRT5⁺ basal cells as in (b) from 3 biological replicates, error bars=SEM, *=p<0.05 f, Schema outlining FACS analysis from freshly fixed human distal lung with anti-KRT5 (intracellular) and monoclonal anti-TNFRSF12A (cell surface) (top), or sequential FACS schema on viable cells freshly dissociated human distal lung to isolate EPCAM⁺ITGA6⁺ITGB4⁺ cells followed by fractionation into TNFRSF12A^{hi} or TNFRSF12A^{neg} subsets. All FACS populations were pre-gated on live singlets. g, Representative whole mount KRT5 staining and quantitation of clonogenic organoid formation from FACS-isolated TNFRSF12A^{lo} and TNFRSF12A^{hu}EPCAM⁺ITGA6⁺ITGB4⁺ cells as in (f) after 14 days culture, scale bar=500 μm. Quantitation represents organoid growth per 1000 FACS isolated cells; data are from cultures from five unique individuals each with a mean of 3 technical replicates, *=<0.05. h, H&E and immunostaining of organoids generated from the TNFRSF12A^{hi} fraction of freshly dissociated EPCAM⁺ITGA6⁺ITGB4⁺ human distal lung cells. Whole mount staining for SCGB1A1 and acetylated tubulin (AcTUB) at the indicated culture time points. Scale bar=50 μm. i, H&E and immunostaining of SCGB1A1 or acetylated tubulin (AcTUB) in 2D air liquid interface cultures initiated from basal cell organoids cultured from the TNFRSF12A^{hi} fraction of EPCAM⁺ITGA6⁺ITGB4⁺ freshly dissociated human distal lung cells. Scale bar=50 μm.

FIG. 5. SARS-CoV-2 and influenza H1N1 infection of distal lung organoids. a-b, Mixed distal lung organoid modeling of H1N1 influenza infection. a, Merged transmission and GFP confocal images of purified basal (left) and purified AT2 organoids (right) 12 hours after infection with PR8-GFP H1 N1 influenza, quantified by FACS for % GFP⁺ cells. Scale bars=50 μm. High resolution images are provided in Supplementary Data. b, Viral genome quantitation over time of mixed distal lung organoid culture supernatants subjected to initial infection of wild-type H1N1 at an estimated multiplicity of infection (MOI) of 0.01, qRT-PCR, 3 biological replicates each with two technical duplicates, error bars=SEM, *=p 21 .05. c, scRNA-seq plots of ACE2and TMPRSS2 gene expression in mixed distal lung organoids from FIG. 1q-r. d, Depiction of formation of apical-out lung organoids. Left: Diagram and representative confocal microscopy showing reorganization of microfilaments (phalloidin) and acetylated microtubules (AcTUB) upon ECM removal. Scale bar=10 μm. Right: Confocal microscopy depicting differentiated cells (AcTUB³⁰ ciliated cells and SCGB1A1⁺ club cells) exposed on the apical surface upon ECM removal. Scale bar=20 μm. e, Confocal microscopy showing apical ACE2 immunofluorescence (yellow arrows) on apical-out basal organoids. Scale ba =10 μm. f, qPCR of SARS-CoV-2 unspliced genomic RNA (left) and spliced subgenomic RNA (right) from infected apical-out distal lung organoids at 72 hours post-infection. n=2 biological replicates. g, Confocal microscopy of double-stranded RNA (dsRNA) immunofluorescence on apical-out human distal lung organoids infected with SARS-CoV-2, mock vs. 48 hours post infection. Scale bar =20 μm. h, Confocal microscopy demonstrating SARS-CoV-2 nucleocapsid protein (NP) immunofluorescence on apical-out human distal lung organoids infected with SARS-CoV-2, mock vs. 96 hours post infection. Scale bar=20 μm. i, Confocal immunofluorescence analysis of SARS-CoV-2 infection of apical-out AT2 organoids with the indicated antibodies at 96 hours post-infection. Scale bar=10 μm. j, Colocalization of SARS-CoV-2 NP and SCGB1A1 immunofluorescence 96 hours post infection of apical-out distal lung organoids. Scale bar=20 μm. k, Cell type specificity of SARS-CoV-2 infection in apical-out distal lung organoids. Immunofluorescence was performed with the indicated antibodies. Top and bottom left: infected club cell adjacent to uninfected ciliated cells. Bottom right: infected cell adjacent to uninfected club cell. Inf=SARS-CoV-2 infected cell. Scale bar=10 μm. In e-k, organoids were everted prior to infection for 6-10 days (basal) and 3 days (AT2).

FIG. 6. Optimization of organoid culture of human distal lung. a, Schematic of culture initiation from human distal lung. b, Brightfield microscopy evaluation of required exogenous growth factors and automated organoid quantitation. c-d, Isolation of purified AT2 organoids. c, Representative FACS plots showing AT2 purification from unfractionated organoid cultures. d, Immunostaining of cytospin of sorted AT2 cells from (c) show high purity (100/100 cells SPC+SCGB1A1−KRT5-); scale bar=50 μM). e-g, Basal organoids in mixed culture progressively form internal lumens which is not associated with apoptosis. e, KRT5 IF, day 26 culture. f. Lumen quantitation, d12 versus d26 culture. g, Absence of apoptosis in d26 basal cell organoid internal lumen, cleaved caspase IF, from FIG. 2k, scale bar=20 μm. h-j, Isolation of purified basal cell organoids via differential sedimentation in Ficoll. h, Schema and enrichment to >90% KRT5+ cells as measured by intracellular KRT5 FACS of sedimented basal organoid cells; scale bar=100 μm. i, Serial time lapse microscopy of sedimented basal organoids reveals spontaneous cavitation within two weeks post passage or within four weeks of culture initiation; scale bar=25 pm. j, Growth factor evaluation for basal organoids after d14 sedimentation, enzymatic dissociation and clonogenic culture. Growth was not affected by the PORCUPINE inhibitor C59 (1 pM). n=3 technical replicates, error bars=SEM, *=p<0.05.

FIG. 7. scRNA-seq of human distal lung organoids reveals reproducible basal, club, and AT2 populations cultured from three individuals. a-c, unsupervised clustering of total cell populations demonstrates consistency in top differentially expressed genes corresponding to basal (KRT5/6), club (SCGB1A1), and AT2 (SFTPC) cells. The epithelial fraction from these cultures ranged from approximately 90-99% of all cells with the remainder being either fibroblasts (VIM+) or mononuclear cells (HLA+, likely alveolar macrophages). d-f, t-SNE visualization and violin plots for marker genes corresponding to each population. Note, a unique population enriched for SPRR genes, which have been described as a marker in squamous metaplasia, were exclusively found in the organoid culture of Lung 3, derived from an individual who was an active smoker.

FIG. 8. Trajectory inference with SPADE. a, SPADE plot of cells where each point represents cell states that are more related on the same or adjacent branches of a minimum spanning tree. Note: AT2 cells exist on a branch distal to basal and club cells, suggesting no lineage hierarchy between AT2, basal, and Club cells. b, SPADE plots of pooled scRNA-seq samples after excluding AT2, VIM+ and HLA+ cells support lineage relationships between basal (blue) and Club (red) populations by Club cell branches emanating from basal cells. c, SPADE plots of Basal 1, Basal 2, and Club populations. d, gene expression of SCGB1A1 shows higher expression in Club versus basal cell lineages as compared to e, KRT5. f, Median gene expression of TNFRSF12A, showing a high (orange outline) and a low (blue outline) within basal cell branches and inferring a potential lineage relationship.

FIG. 9. Human AT2 organoid cells contain lamellar bodies. a, Confocal images of a live AT2 organoid at 67 days of culture labeled with Hoescht nuclear stain and LysoTracker Red DND-99. b, Transmission electron microscopy image of representative AT2 organoid at 28 days of culture. Note apical microvilli (black arrows) and lamellar bodies (red arrows); scale bar=10 μm.

FIG. 10. Subclustering of AT2 cells. a-c, Upon configuring the parameter for the graph based cluster to create more clusters, AT2 populations were initially divided into two populations within the Lung 1 sample (a). However, the same clustering procedure did not sub-divide the AT2 populations in the Lung 2 and Lung 3 samples (b-c), likely due to fewer number of cells. Therefore, for these two samples in particular, we isolated the raw expression of the AT2 cells, and re-analyzed with Seurat and graph-based clustering was solely performed on these cells. The clustering parameter was set such that two clusters would be created and compared the population structure with that in Lung 1. d-h, scRNA-seq analysis of 2,780 cells in organoid culture derived from FACS purified AT2 cells after 89 days of organoid culture from FIG. 2. d-e, Clustering and t-SNE projection of AT2 cells demonstrates a proliferative subcluster (Cluster 1) that is defined by cell cycle genes per GSEA but does not correspond to a specific AT2 subpopulation. f-h, Feature plots of LYZ, MUC5B, and CD74 do not highlight discrete AT2 subpopulations that correspond to those seen in mixed organoid cultures, All plots are log10 expression.

FIG. 11. scRNA-seq identifies an active basal cell subpopulation across three individual patient organoid cultures. a-c, High resolution clustering analysis identifies a reproducible active basal cell subpopulation with significantly higher expression of the surface marker TNFRSF12A, the NOTCH pathway marker HES1, and the proliferation marker MKI67. Modified Kruskal-Wallis Rank Sum Test p-values: TNFRSF12A 4.15×10⁻⁸; HES1 2.4×10⁻¹⁰; MKI67 3.4×10⁻³.

FIG. 12. scRNA-seq analysis of proliferation within the Basal 1 population a, Fine resolution clustering of KRT5+ populations identifies two Basal 1 sub-clusters, Basal 1.1 and 1.2. b, Gene Ontology PANTHER overrepresentation of differentially expressed genes enriched in Basal 1.2 versus 1.1 show the majority of Basal 1.2 processes involve cell cycle (asterisks). c, scRNA-seq subfractionation of cells simultaneously expressing EPCAM, ITGA6 and ITGB4 mRNA from FIG. 3d, according to highest quartile (Hi, 76-100%), medium (Med, 26- 75%) or lowest quartile (Lo, 0-25%) mRNA expression of the indicated transcripts (TNFRSF12A, ITGA6, KRT5 and TP63). Amongst the Lo, Med and Hi fractions of each Basal 1 marker, the cell fraction positive for the indicated cell cycle mRNAs (MKI67, MYBL2, PLK1, BUB1, E2F1, FOXM1) are plotted in the bar graphs. TNFRSF12A mRNA expression strongly enriches for cell cycle mRNAs over the progression from Lo→Med→Hi, which is not observed for ITGA6, KRT5 and TP63 mRNA expression does not. For TNFRSF12A and ITGA6, Hi, Med, and Lo correspond to the top 25%, mid 50%, and bottom 25% quartiles respectively. However, for KRT5 and TP63, a significant number of cells expressed 0 UMI, so we defined Lo as cells with zero expression, and Hi and Med as the top 50% and bottom 50% of cells with non-zero expression. Thus, KRT5 has Hi (33.7%), Med (33.7%), and Lo (32.6%), and TP63 has Hi (12.8%), Med (12.8%), and Lo (74.4%).

FIG. 13. Evaluation of Basal 1 lineage relationship to Basal 2 and the influence of NOTCH signaling on Basal 1 renewal and differentiation. a, Isolation of Basal 1 and Basal 2 via differential sedimentation of KRT5+cells followed by FACS sorting of EPCAM+ITGA6+ITGB4+TNFRSF12A+ (Basal 1) versus EPCAM-ITGA6-ITGB4-TNFRSF12A-(Basal 2). b, Intracellular FACS measurement of KRT5+ protein expression in Basal 1 and 2 fractions from (a). c, representative brightfield of day 14 cultures from (a-b). d, quantitation of 3 biologic replicates from (a-c) (*** p<0.001 two tailed t-test). e, qPCR measurement of two differentially upregulated Basal 2 genes from the three scRNA-seq biological replicates (SPRR1B, TMSB4X) after prolonged culture of FACS isolated Basal 1 cells. Data are relative mean +/− SEM of cultures from three individuals, **=p<0.01. f, RNA FISH demonstrating TMSB4X and SPRR1B cellular transcripts within organoids originating from Basal 1 cells (arrows), scale bar=25 μm. g, KRT5 immunostaining and SFTPC and SCGB1A1 RNA FISH of FACS isolated TNFRSF12A^{hi} Basal 1 cells under vehicle, NOTCH agonism (JAG1 peptide), or NOTCH antagonism with the Delta-like ligand mutant 4 (DLL4E12) or the gamma secretase inhibitor DBZ; scale bar=50 μm. h, Fluorescent quantitation of resazurin dye reduction to estimate relative cellular proliferation in (a), data represent mean and SEM of cultures from five individuals, * p<0.05 two-tailed Student's t test. i, Quantitation of SCGB1A1 and SFTPCgene expression by RNA FISH in the context of NOTCH agonism or antagonism, ** p<0.01, *** p<0.001 Student's t-test.

FIG. 14. Characterization of mixed distal lung organoid culture for influenza infection modeling. a-b, Lectin staining with M. amurensis (a 2-3) and S. nigra (a 2-6) lectins or no lectin negative controls to characterize sialic acid residues which serve as surface molecules for influenza virus host cell entry. Scale bar=25 μm. c, Dose response curves for two different classes of antiviral drugs on influenza infectivity and replication. As expected, the nucleoside analog FdC demonstrated an IC50 of 340 nM as compared to zanamivir, which did not exhibit a dose response curve and an IC50, a neuraminidase antagonist which only impairs viral shedding, but not infectivity and replication. N=3 technical replicates. d, Fluorescence micrograph of multiwell screening of selected various antiviral agents after H1N1 PR8-GFP organoid infection in 48 well format. FdC=nucleoside analog 2′-deoxy-2′-fluorocytidine. Cpd=compound #.

FIG. 15. Apical-out polarization and multi-lineage differentiation of distal lung organoids upon suspension culture. a-d, Eversion and accelerated ciliary differentiation of apical-out basal organoids. a, Confocal 3D sections (top panels) and surface reconstructions (bottom panels) of apical-out lung organoids at different days after ECM removal. At day 0 (d0) microfilament (green, phalloidin) and microtubule (red, acetylated tubulin) organization is not polarized. Junctional strands (ZO-1, white) are discontinuous and found between cells inside of organoids. By day 2 in suspension (d2) ZO-1 (white) forms junctional rings in the apical periphery of each cell facing the external side of the organoids and the actin cytoskeleton forms microvilli (green) facing outward (apical-out polarity). Also at d2 some cells initiate microtubule polarization and formation of tufts of cilia. By live-cell imaging cilia become motile by day 5. By day 5 (d5) many more cells have differentiated into multiciliated respiratory epithelium with motile cilia facing outwards. Mature motile cilia can be observed for several weeks, example at day 14 (d14), (Supplemental Video 3). b, 3D confocal reconstruction of an organoid embedded in ECM consisting mostly of basal stem cells (KRTS+, white). c, As apical-out polarity is established and ciliagenesis begins, KRT5+ cells decrease in abundance. Basal cells are found underneath the polarized epithelium. d, SCGB1A1 Club cells with apical-out polarity can are present on the exterior everted surface. In all panels nuclei are stained blue with DAPI, and actin microfilament organization visualized with phalloidin (green). Scale bars=10 μm. e-g, Prolonged suspension culture of AT2 organoids (day 10 post-eversion) induces apical-out polarization and AT1 differentiation. e, Optical sections through alveolar-derived organoids after 10 days in suspension culture show decreased abundance of AT2 cells while individual cuboidal cells begin to express the AT1 marker HT1-56 (red), a transmembrane protein specific to the apical membrane of alveolar type 1 pneumocytes (AT1). f-g, Side views of alveolar organoids after 10 days of suspension culture reveal thin AT1 cells with apical junctional complexes facing outwards (apical-out) and expression of HT1-56 on the apical membrane.

### DETAILED DESCRIPTION

Using the methods disclosed herein it is demonstrated SARS-CoV-2 infection of distal lung organoids, either alveoli or terminal bronchioles, and demonstrated Club cells in terminal bronchioles as a novel SARS-CoV-2 target cell.

As mixed cultures, the distal lung organoids grow as intermingled small cystic (alveolar, SFTPC+) and larger solid (basal cell, KRT5+) colonies for greater than 6 months. The mixed cultures can be fractionated into pure basal and pure alveolar organoids. The pure basal organoids spontaneously differentiate over months to Club and ciliated lineages and crucially express ACE2, the SARS-CoV-2 receptor. The alveolar organoids possess characteristic lamellar bodies and also express ACE2, and by scRNA-seq are pure alveolar type 2 (AT2) cells. Notably, both basal and alveolar cells are readily infected with influenza H1N1-GFP reporter strains. These human distal lung organoids form the basis for SARS-CoV-2 infection.

The peripheral 1 cm of the lung, containing alveoli and distal bronchioles, is enzymatically processed and grown as submerged domes in an extracellular matrix gel. This has been performed from surgical samples and routinely yields vigorously growing mixed organoids containing both basal and alveolar colonies. Alternatively, pure alveolar or basal organoids can be grown following by HTII-280/Lysotracker FACS and sedimentation, respectively. The alveolar organoids are pure alveolar type 2 cells and the basal organoids are primarily basal cells but differentiate to club and ciliated cells. Culture occurs in submerged extracellular matrix domes (BME2) with EGF and Noggin media layered on top. To obtain apical-basal polarity reversal, the organoids are removed from the extracellular matrix and placed into suspension culture. This leads to spontaneous relocation of differentiated cells from the lumen of the organoid (where they displayed their apical aspects facing inwards towards the lumen), to the organoid exterior (where their apical aspect now faces outwards towards the surrounding tissue culture media and extracellular matrix).

Concurrently, the ACE2 receptor for SARS-CoV-2 now becomes expressed on cells lining the exterior surface of the organoid, on the outwards-facing apical aspect, allowing facile infection by SARS-CoV-2 added to the tissue culture medium. Under these conditions, both alveolar and distal bronchiolar organoids are infected, with alveolar type 2, club and other cells targeted. This method thus allows apical infection of distal lung organoid cells by SARS-CoV-2 including alveolar type 2 and club cells, which has not previously been possible, with extension to other lung pathogens including bacteria and viruses.

In the description that follows, a number of terms conventionally used in the field of cell culture are utilized extensively. In order to provide a clear and consistent understanding of the specification and claims, and the scope to be given to such terms, the following definitions are provided.

The term “cell culture” or “culture” means the maintenance of cells in an artificial, in vitro environment. It is to be understood, however, that the term “cell culture” is a generic term and may be used to encompass the cultivation not only of individual cells, but also of tissues or organs.

The term “culture system” is used herein to refer to the culture conditions in which the subject explants are grown that promote prolonged tissue expansion with proliferation, multilineage differentiation and recapitulation of cellular and tissue ultrastructure.

“Gel substrate”, as used herein has the conventional meaning of a semi-solid extracellular matrix. Gel described here in includes without limitations, collagen gel, matrigel, extracellular matrix proteins, fibronectin, collagen in various combinations with one or more of laminin, entactin (nidogen), fibronectin, and heparin sulfate; human placental extracellular matrix.

By “container” is meant a glass, plastic, or metal vessel that can provide an aseptic environment for culturing cells.

The term “explant” is used herein to mean a piece of tissue and the cells thereof originating from mammalian tissue that is cultured in vitro, for example according to the methods of the invention. The mammalian tissue from which the explant is derived may obtained from an individual, i.e. a primary explant, or it may be obtained in vitro, e.g. by differentiation of induced pluripotent stem cells.

The term “organoid” is used herein to mean a 3-dimensional growth of mammalian cells in culture that retains characteristics of the tissue in vivo, e.g. prolonged tissue expansion with proliferation, multilineage differentiation, recapitulation of cellular and tissue ultrastructure, etc. A primary organoid is an organoid that is cultured from an explant, i.e. a cultured explant. A secondary organoid is an organoid that is cultured from a subset of cells of a primary organoid, i.e. the primary organoid is fragmented, e.g. by mechanical or chemical means, and the fragments are replated and cultured. A tertiary organoid is an organoid that is cultured from a secondary organoid, etc.

The phrase “mammalian cells” means cells originating from mammalian tissue. Typically, in the methods of the invention pieces of tissue are obtained surgically and minced to a size less than about 1 mm³, and may be less than about 0.5 mm³, or less than about 0.1 mm³. “Mammalian” used herein includes human, equine, bovine, porcine, canine, feline, rodent, e.g. mice, rats, hamster, primate, etc. “Mammalian tissue cells” and “primary cells” have been used interchangeably.

“Tissue-specific stem cells” is used herein to refer to multipotent stem cells that reside in a particular tissue and are capable of clonal regeneration of cells of the tissue in which they reside, for example the ability of hematopoietic stem cells to reconstitute all hematopoietic lineages, or the ability of neuronal stem cells to reconstitute all neuronal/glial lineages. “Progenitor cells” differ from tissue-specific stem cells in that they typically do not have the extensive self-renewal capacity, and often can only regenerate a subset of the lineages in the tissue from which they derive, for example only lymphoid or erythroid lineages in a hematopoietic setting, or only neurons or glia in the nervous system.

Culture conditions of interest provide an environment permissive for differentiation, in which the complex cell system from an explant cells will proliferate, differentiate, or mature in vitro. Such conditions may also be referred to as “differentiative conditions”. Features of the environment include the medium in which the cells are cultured, any growth factors or differentiation-inducing factors that may be present, and a supporting structure (such as a substrate on a solid surface) if present.

The term “multi-lineage differentiation markers” means differentiation markers characteristic of different cell-types. These differentiation markers can be detected by using an affinity reagent, e.g. antibody specific to the marker, by using chemicals that specifically stain a cell type, etc as known in the art.

“Ultrastructure” refers to the three-dimensional structure of a cell or tissue observed in vivo. For example, the ultrastructure of a cell may be its polarity or its morphology in vivo, while the ultrastructure of a tissue would be the arrangement of different cell types relative to one another within a tissue.

The term “candidate cells” refers to any type of cell that can be placed in co-culture with the tissue explants described herein. Candidate cells include without limitations, mixed cell populations, ES cells and progeny thereof, e.g. embryoid bodies, embryoid-like bodies, embryonic germ cells.

The term “candidate agent” means any oligonucleotide, polynucleotide, siRNA, shRNA, gene, gene product, peptide, antibody, small molecule or pharmacological compound that is introduced to an explant culture and the cells thereof as described herein to assay for its effect on the explants.

The term “contacting” refers to the placing of candidate cells or candidate agents into the explant culture as described herein. Contacting also encompasses co-culture of candidate cells with tissue explants for at least 1 hour, or more than 2 hrs or more than 4 hrs in culture medium prior to placing the tissue explants in a semi-permeable substrate. Alternatively, contacting refers to injection of candidate cells into the explant, e.g. into the lumen of an explant.

“Screening” refers to the process of either co-culturing candidate cells with or adding candidate agents to the explant culture described herein and assessing the effect of the candidate cells or candidate agents on the explant. The effect may be assessed by assessing any convenient parameter, e.g. the growth rate of the explant, the presence of multilineage differentiation markers indicative of stem cells, etc. The effect of candidate cells or candidate agents on the explant can be further evaluated by assaying the explant for long-term reconstitutive activity by serial in vitro passage, as well as by in vivo transplantation.

Culture systems and methods are provided. By long term culture, it is meant continuous growth of the explant for extended periods of time, e.g. for 15 days or more, for 1 month or more, for 2 months or more, for 3 months or more, for 6 months or more, or up to a year, or more. By continuous growth, it is meant sustained viability, organization, and functionality of the tissue. For example, unless experimentally modified, proliferating cells in a tissue explant that undergoes continuous growth in the culture systems of the present application will continue to proliferate at their natural rate, while non-proliferative, e.g. differentiated, cells in the tissue explant will remain in a quiescent state. Because of this, explants cultured by the subject methods are referred to as “organoids”.

In some embodiments, tissue, i.e. primary tissue, is obtained from a mammalian organ. The tissue may be from any mammalian species, e.g. human, equine, bovine, porcine, canine, feline, rodent, e.g. mice, rats, hamster, primate, etc. The mammal may be of any age, e.g. a fetus, neonate, juvenile, adult. The following are some non-limiting examples of tissues that may be obtained for the purposes of preparing organoids:

Tissue may be obtained by any convenient method, e.g. by biopsy, e.g. during endoscopy, during surgery, by needle, etc., and is typically obtained as aseptically as possible. Upon removal, tissue is immersed in ice-cold buffered solution, e.g. PBS, Ham's F12, MEM, culture medium, etc. Pieces of tissue are minced to a size less than about 1 mm³, and may be less than about 0.5 mm³, or less than about 0.1 mm³. The minced tissue is mixed with a gel substrate, e.g. a collagen gel solution, e.g. Cellmatrix type I-A collagen (Nitta Gelatin Inc.); a matrigel solution, etc.

Explants cultured in this way may be sustained for over a year at physiological temperatures, e.g. 37° C., in a humidified atmosphere of, e.g. 5% CO₂ in air. Medium is changed about every 10 days or less, e.g. about 1, 2, or 3 days, sometimes 4, 5, or 6 days, in some instances 7, 8, 9, 10, 11 or 12 days, usually as convenient.

The continued growth of explants may be confirmed by any convenient method, e.g. phase contrast microscopy, stereomicroscopy, histology, immunohistochemistry, electron microscopy, etc. In some instances, cellular ultrastructure and multi-lineage differentiation may be assessed. Ultrastructure of the intestinal explants in culture can be determined by performing Hematoxylin-eosin staining, PCNA staining, electron microscopy, and the like using methods known in the art. Multi-lineage differentiation can be determined by performing labeling with antibodies to terminal differentiation markers, e.g. as described in greater detail below. Antibodies to detect differentiation markers are commercially available from a number of sources.

In some embodiments, the cells in the cultured explants may be experimentally modified. For example, the explant cells may be modified by exposure to viral or bacterial pathogens, e.g. to develop a reagent for experiments to assess the anti-viral or anti-bacterial effects of therapeutic agents. The explant cells may be modified by altering patterns of gene expression, e.g. by providing reprogramming factors to induce pluripotency or otherwise alter differentiation potential, or to determine the effect of a gain or loss of gene activity on the ability of cells to form an explant culture or on the ability of cells to undergo tumor transformation. The explant cells may be modified such that they are transformed with growth factors or cytokines or other genes to modulate viral infection phenotypes on immune cells or intestinal epithelial cells.

Experimental modifications may be made by any method known in the art, for example, as described below with regard to methods for providing candidate agents that are nucleic acids, polypeptides, small molecules, viruses, etc. to explants and the cells thereof for screening purposes.

As used herein, the term “infection” refers to any state in at least one cell of an organism (i.e., a subject) is infected by an infectious agent. As used herein, the term “infectious agent” refers to a foreign biological entity, i.e. a pathogen, that induces increased CD47 expression in at least one cell of the infected organism. For example, infectious agents include, but are not limited to bacteria, viruses, protozoans, and fungi. Intracellular pathogens are of particular interest. Infectious diseases are disorders caused by infectious agents. Some infectious agents cause no recognizable symptoms or disease under certain conditions, but have the potential to cause symptoms or disease under changed conditions.

Pathogens of interest include respiratory pathogens, e.g. influenza, rhinovirus, adenovirus, coronavirus such as SARS-CoV1, SARS-CoV2, MERS-CoV, etc.; tuberculosis, Legionella, Yersinia; and the like.

Cultures

Single cell suspensions of lung tissue are cultured within a droplet of collagen/laminin extracellular matrix without exogenous feeder cells. In some embodiments, proximal cell types are excluded from the culture, for example by selecting only peripheral lung tissue underlying the mesothelium. In some embodiments, to generate pure clonogenically-derived AT2 organoids, viable AT2 cells are purified from mixed distal lung organoids, e.g. by flow cytometry, and individual cells are expanded. The cells can be cultured for extended period of time, e.g. from about 3 to about 12 months, or more.

To isolate distal airway cells, lung parenchyma 1 cm from the visceral pleura is dissociated and resuspended in lung organoid media, comprising an effective dose of an EGF agonist, a BMP antagonist, and may comprise a TGF-β inhibitor. Cells are resuspended in a matrix culture.

Basal cell organoids in mixed distal lung culture initially formed solid KRT5+ masses, but developed single but occasionally multiple lumens. The basal stem cell marker KRT5 was specifically excluded from the differentiated lumen zone, but is otherwise diffusely expressed. Pure basal cell cultures can be established by using density sedimentation to remove cystic AT2 organoids, leaving behind solid basal organoids which were then disaggregated and regrown from single cell suspensions.

Single cells can be isolated from organoids and selected by flow cytometry, e.g. for expression of one or more of TNFRSF12A; EPCAM, ITGA6/ITGB4. The isolated cells can be seeded in extracellular matrix and cultured to organoids by a method as done for the initial organoids.

Unfractionated cultures containing AT2, basal, and club cell types at 2-3 weeks can be infected with a pathogen, e.g. influenza. Productivity of pandemic H1 N1 virus infection can be determined by various methods known in the art, e.g. determined by qPCR.

In some embodiments the organoids have everted polarity. 3D basal and alveolar organoids are typically oriented with the basolateral surface oriented outwards, i.e. facing the extracellular matrix substratum, which can hinder infection of the apical ACE2-expressing luminal surface. The cultures can be everted by removal from extracellular matrix gel and growth in suspension, robustly generating organoids with their apical surfaces oriented outward. Within from about 1 to about 3 days, around about 2 days, non-polarized organoids reorganize into apical-out epithelial spheroids with microvilli, apical junctions, and some motile cilia facing the organoid exterior. Everted organoids display outwardly facing club cells with apical secretory granules. SARS-CoV-2 readily infected apical-out mixed distal lung organoids, with direct SARS-CoV-2 infection of AT2 cells, and club cells as a novel target population.

The medium for culture may comprise an effective dose of an EGF agent, e.g. human EGF protein, agonist antibodies, etc.; and an effective dose of a BMP antagonist, including without limitation, NOGGIN. EGF and NOGGIN are sufficient for clonal AT2 organoid proliferation. Exogenous WNT-3A and RSPONDIN-1 did not enhance growth. The medium may comprise extracellular matrix, unless it is an everted culture. The medium may comprise an inhibitor of TGFβ.

As used herein, the term “BMP” refers to the family of bone morphogenetic proteins, which reference sequence may be found in Genbank, for example BMP-2 accession number NP_001191. Antagonists include antibodies and fragments thereof that block activity of the cognate BMP receptor.

Inhibitors (antagonists) of the BMP pathway include but are not limited to, e.g., NOGGIN, CHORDIN, LDN-193189 (4-[6-[4-(1-Piperazinyl)phenyl]pyrazolo[1,5-a]pyrimidin-3-yl]-quinoline hydrochloride), DMH1 (4-[6-[4-(1-Methylethoxy)phenyl]pyrazolo[1,5-a]pyrimidin-3-yl]-quinoline), Dorsomorphin (6-[4-[2-(1-Piperidinyl)ethoxy]phenyl]-3-(4-pyridinyl)-pyrazolo[1,5-a]pyrimidine dihydrochloride), K 02288 (3-[(6-Amino-5-(3,4,5-trimethoxyphenyl)-3-pyridinyl]phenol), ML 347 (5-[6-(4-Methoxyphenyl)pyrazolo[1,5-a]pyrimidin-3-yl]quinoline), DMH-1, antibodies to BMPs and BMP receptors, BMP inhibitory nucleic acids, and the like. In some instances, the agents, as described above include, e.g., those that are commercially available, e.g., from such suppliers such as Tocris Bioscience (Bristol, UK), Sigma-Aldrich (St. Louis, Mo.), Santa Cruz Biotechnology (Santa Cruz, Calif.), and the like.

Inhibitors of the TGF-beta pathway include but are not limited to, e.g., A-83-01 (3-(6-Methyl-2-pyridinyl)-N-phenyl-4-(4-quinolinyl)-1H-pyrazole-1-carbothioamide), D4476 (4-[4-(2,3-Dihydro-1,4-benzodioxin-6-yl)-5-(2-pyridinyl)-1H-imidazol-2-yl]benzamide), GW 788388 (4-[4-[3-(2-Pyridinyl)-1H-pyrazol-4-yl]-2-pyridinyl]-N-(tetrahydro-2H-pyran-4-yl)-benzamide), LY 364947 (4-[3-(2-Pyridinyl)-1 H-pyrazol-4-yl]-quinoline), RepSox (2-(3-(6-Methylpyridine-2-yl)-1H-pyrazol-4-yl)-1,5-naphthyridine), SB431542 (4-[4-(1,3-benzodioxol-5-yl)-5-(2-pyridinyl)-1H-imidazol-2-yl]benzamide), SB-505124 (2-[4-(1,3-Benzodioxol5-yl)-2-(1,1-dimethylethyl)-1H-imidazol-5-yl]-6-methyl-pyridine), SB 525334 (6-[2-(1,1-Dimethylethyl)-5-(6-methyl-2-pyridinyl)-1H-imidazol-4-yl]quinoxaline), SD208 (2-(5-Chloro-2-fluorophenyl)-4-[(4-pyridyl)amino]pteridine), ITD1 (4-[1,1′-Biphenyl]-4-yl-1,4,5,6,7,8-hexahydro-2,7,7-trimethyl-5-oxo-3-quinolinecarboxylic acid ethyl ester), DAN/Fc, antibodies to TGF-beta and TGF-beta receptors, TGF-beta inhibitory nucleic acids, and the like.

The lung tissue cell suspension may be contacted with agents by any convenient means.

Generally the agents are added to culture media, as described herein, within which cells of the instant disclosure are grown or maintained, such that the agent is present, in contact with the cells, at an effective concentration to produce the desired effect.

The effective concentration of an agent will vary and will depend on the agent. In addition, in some instances, the effective concentration may also depend on the cells being induced, the culture condition of the cells, other agents co-present in the culture media, etc. As such, the effective concentration of agents may range from 1 ng/mL to 10 μg/mL or more, including but not limited to, e.g., 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, 25 ng/mL, 26 ng/mL, 27 ng/mL, 28 ng/mL, 29 ng/mL, 30 ng/mL, 31 ng/mL, 32 ng/mL, 33 ng/mL, 34 ng/mL, 35 ng/mL, 36 ng/mL, 37 ng/mL, 38 ng/mL, 39 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 1-5 ng/mL, 1-10 ng/mL, 1-20 ng/mL , 1-30 ng/mL, 1-40 ng/mL, 1-50 ng/mL, 5-10 ng/mL, 5-20 ng/mL, 10-20 ng/mL, 10-30 ng/mL, 10-40 ng/mL, 10-50 ng/mL, 20-30 ng/mL, 20-40 ng/mL, 20-50 ng/mL, 30-40 ng/mL, 30-50 ng/mL, 40-50 ng/mL, 1-100 ng/mL, 50-100 ng/mL, 60-100 ng/mL, 70-100 ng/mL, 80-100 ng/mL, 90-100 ng/mL, 10-100 ng/mL, 50-200 ng/mL, 100-200 ng/mL, 50-300 ng/mL, 100-300 ng/mL, 200-300 ng/mL, 50-400 ng/mL, 100-400 ng/mL, 200-400 ng/mL, 300-400 ng/mL, 50-500 ng/mL, 100-500 ng/mL, 200-500 ng/mL, 300-500 ng/mL, 400 to 500 ng/mL, 0.001-1 μg/mL, 0.001-2 μg/mL, 0.001-3 μg/mL, 0.001-4 μg/mL, 0.001-5 μg/mL, 0.001-6 μg/mL, 0.001-7 μg/mL, 0.001-8 μg/mL, 0.001-9 μg/mL, 0.001-10 μg/mL, 0.01-1 μg/mL, 0.01-2 μg/mL, 0.01-3 μg/mL, 0.01-4 μg/mL, 0.01-5 μg/mL, 0.01-6 μg/mL, 0.01-7 μg/mL, 0.01-8 μg/mL, 0.01-9 μg/mL, 0.01-10 μg/mL, 0.1-1 μg/mL, 0.1-2 μg/mL, 0.1-3 μg/mL, 0.1-4 μg/mL, 0.1-5 μg/mL, 0.1-6 μg/mL, 0.1-7 μg/mL, 0.1-8 μg/mL, 0.1-9 μg/mL, 0.1-10 μg/mL, 0.5-1 μg/mL, 0.5-2 μg/mL, 0.5-3 μg/mL, 0.5-4 μg/mL, 0.5-5 μg/mL, 0.5-6 μg/mL, 0.5-7 μg/mL, 0.5-8 μg/mL, 0.5-9 μg/mL, 0.5-10 μg/mL, and the like.

### Utility

Organoids prepared by the subject methods may be used in basic research, e.g. to better understand the basis of disease, and in drug discovery, e.g. as reagents in screens such as those described further below, and for diagnostic purposes. Organoids are also useful for assessing the pharmacokinetics and pharmacodynamics of an agent, e.g. the ability of a mammalian tissue to absorb an active agent, the cytotoxicity of agents on primary mammalian tissue or on oncogenic mammalian tissue, etc.

### Screening Methods

In some aspects of the invention, methods and culture systems are provided for screening candidate agents or cells for an activity of interest. In these methods, candidate agents or cells are screened for their effect on cells in the organoids of the invention. Organoids of interest include those comprising unmodified cells, and those comprising experimentally modified cells.

The effect of an agent or cells is determined by adding the agent or cells to the cells of the cultured explants as described herein, usually in conjunction with a control culture of cells lacking the agent or cells. The effect of the candidate agent or cell is then assessed by monitoring one or more output parameters. Parameters are quantifiable components of explants or the cells thereof, particularly components that can be accurately measured, in some instances in a high throughput system. For example, a parameter of the explant may be the growth, differentiation, gene expression, proteome, phenotype with respect to markers etc. of the explant or the cells thereof, e.g. any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

In some embodiments, candidate agent or cells are added to the cells within the intact organoid. In other embodiments, the organoids are dissociated, and candidate agent or cells is added to the dissociated cells. The cells may be freshly isolated, cultured, genetically altered as described above; or the like. The cells may be environmentally induced variants of clonal cultures: e.g. split into independent cultures and grown into organoids under distinct conditions, for example with or without pathogen; in the presence or absence of other cytokines or combinations thereof. The manner in which cells respond to an agent, particularly a pharmacologic agent, including the timing of responses, is an important reflection of the physiologic state of the cell.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992).

Candidate agents of interest for screening also include nucleic acids, for example, nucleic acids that encode siRNA, shRNA, antisense molecules, or miRNA, or nucleic acids that encode polypeptides. Many vectors useful for transferring nucleic acids into target cells are available. The vectors may be maintained episomally, e.g. as plasmids, minicircle DNAs, virus-derived vectors such cytomegalovirus, adenovirus, etc., or they may be integrated into the target cell genome, through homologous recombination or random integration, e.g. retrovirus derived vectors such as MMLV, HIV-1, ALV, etc. Vectors may be provided directly to the subject cells. In other words, the pluripotent cells are contacted with vectors comprising the nucleic acid of interest such that the vectors are taken up by the cells.

Methods for contacting cells with nucleic acid vectors, such as electroporation, calcium chloride transfection, and lipofection, are well known in the art. Alternatively, the nucleic acid of interest may be provided to the subject cells via a virus. In other words, the pluripotent cells are contacted with viral particles comprising the nucleic acid of interest. Retroviruses, for example, lentiviruses, are particularly suitable to the method of the invention. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the subject CD33+ differentiated somatic cells are targeted by the packaged viral particles. Methods of introducing the retroviral vectors comprising the nucleic acid encoding the reprogramming factors into packaging cell lines and of collecting the viral particles that are generated by the packaging lines are well known in the art.

Vectors used for providing nucleic acid of interest to the subject cells will typically comprise suitable promoters for driving the expression, that is, transcriptional activation, of the nucleic acid of interest. This may include ubiquitously acting promoters, for example, the CMV-b-actin promoter, or inducible promoters, such as promoters that are active in particular cell populations or that respond to the presence of drugs such as tetracycline. By transcriptional activation, it is intended that transcription will be increased above basal levels in the target cell by at least about 10 fold, by at least about 100 fold, more usually by at least about 1000 fold. In addition, vectors used for providing reprogramming factors to the subject cells may include genes that must later be removed, e.g. using a recombinase system such as Cre/Lox, or the cells that express them destroyed, e.g. by including genes that allow selective toxicity such as herpesvirus TK, bcl-xs, etc

Candidate agents of interest for screening also include polypeptides. Such polypeptides may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like.

If the candidate polypeptide agent is being assayed for its ability to inhibit aggregation signaling intracellularly, the polypeptide may comprise the polypeptide sequences of interest fused to a polypeptide permeant domain. A number of permeant domains are known in the art and may be used in the non-integrating polypeptides of the present invention, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK. As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 Apr; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

If the candidate polypeptide agent is being assayed for its ability to inhibit aggregation signaling extracellularly, the polypeptide may be formulated for improved stability. For example, the peptides may be PEGylated, where the polyethyleneoxy group provides for enhanced lifetime in the blood stream. The polypeptide may be fused to another polypeptide to provide for added functionality, e.g. to increase the in vivo stability. Generally such fusion partners are a stable plasma protein, which may, for example, extend the in vivo plasma half-life of the polypeptide when present as a fusion, in particular wherein such a stable plasma protein is an immunoglobulin constant domain. In most cases where the stable plasma protein is normally found in a multimeric form, e.g., immunoglobulins or lipoproteins, in which the same or different polypeptide chains are normally disulfide and/or noncovalently bound to form an assembled multichain polypeptide, the fusions herein containing the polypeptide also will be produced and employed as a multimer having substantially the same structure as the stable plasma protein precursor. These multimers will be homogeneous with respect to the polypeptide agent they comprise, or they may contain more than one polypeptide agent.

The candidate polypeptide agent may be produced from eukaryotic produced by prokaryotic cells, it may be further processed by unfolding, e.g. heat denaturation, DTT reduction, etc. and may be further refolded, using methods known in the art. Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine. The polypeptides may have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The candidate polypeptide agent may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. Alternatively, the candidate polypeptide agent may be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

In some cases, the candidate polypeptide agents to be screened are antibodies. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be either polyclonal antibodies or monoclonal antibodies. Antibodies are typically provided in the media in which the cells are cultured.

Candidate agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by adding the agent to at least one and usually a plurality of explant or cell samples, usually in conjunction with explants not contacted with the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow-through method. Alternatively, the agents can be injected into the explant, e.g. into the lumen of the explant, and their effect compared to injection of controls.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the growth rate.

Screens for agents to prevent or treat disease. Other examples of screening methods of interest include methods of screening a candidate agent for an activity in treating or preventing a disease. In such embodiments, the explant models the disease, e.g. the explant may have been obtained from a diseased tissue, or may be experimentally modified to model the disease by, e.g., genetic mutation. Parameters such as explant growth, cell viability, cell ultrastructure, tissue ultrastructure, etc. find particular use as output parameters in such screens.

Screens to determine the pharmacokinetics and pharmacodynamics of agents. Other examples include methods of screening a candidate agent for toxicity to tissue. In these applications, the cultured explant is exposed to the candidate agent or the vehicle and its growth and viability is assessed. In these applications, analysis of the ultrastructure of the explants is also useful.

### High Throughput Screens

In some aspects of the invention, methods and culture systems are provided for screening candidate agents in a high-throughput format. By “high-throughput” or “HT”, it is meant the screening of large numbers of candidate agents or candidate cells simultaneously for an activity of interest. By large numbers, it is meant screening 20 more or candidates at a time, e.g. 40 or more candidates, e.g. 100 or more candidates, 200 or more candidates, 500 or more candidates, or 1000 candidates or more.

In some embodiments, the high throughput screen will be formatted based upon the numbers of wells of the tissue culture plates used, e.g. a 24-well format, in which 24 candidate agents (or less, plus controls) are assayed; a 48-well format, in which 48 candidate agents (or less, plus controls) are assayed; a 96-well format, in which 96 candidate agents (or less, plus controls) are assayed; a 384-well format, in which 384 candidate agents (or less, plus controls) are assayed; a 1536-well format, in which 1536 candidate agents (or less, plus controls) are assayed; or a 3456-well format, in which 3456 candidate agents (or less, plus controls) are assayed. High throughput screens formatted in this way may be achieved by using, for example, transwell inserts. Transwell inserts are wells with permeable supports, e.g. microporous membranes, that are designed to fit inside the wells of a multi-well tissue culture dish. In some instances, the transwells are used individual. In some instances, the transwells are mounted in special holders to allow for automation and ease of handling of multiple transwells at one time.

To achieve the numbers of organoids necessary to perform a high-throughput screen, a primary organoid (that is, an organoid that has been cultured directly from tissue fragments) is dissociated into a single cell suspension and replated across multiple transwells to generate secondary organoids in a multiwell format. Dissociation may be by any convenient method, e.g. manual treatment (trituration), or chemical or enzymatic treatment with, e.g. EDTA, trypsin, papain, etc. that promotes dissociation of cells in tissue. The dissociated organoid cells are then replated in transwells at a density of 10,000 or more cells per 96-well transwell, e.g. 20,000 cells or more, 30,000 cells or more, 40,000 cells or more, or 50,000 cells or more. Additional iterations of dissociation and plating may be performed to achieve the desired numbers samples of organoids to be treated with agent.

In some embodiments, the secondary (or tertiary, etc.) organoids may be cultured first, after which candidate agents or cells are added to the organoid cultures and parameters reflective if a desired activity are assessed. In other embodiments, the candidate agents or cells are added to the dissociated cells at replating. This latter paradigm may be particularly useful for example for assessing candidate agents/cells for an activity that impacts the differentiation of cells of the developing organoid. Any one or more of these steps may be automated as convenient, e.g. robotic liquid handling for the plating of explants, addition of medium, and/or addition of candidate agents; robotic detection of parameters and data acquisition; etc.

The following examples are put forth so as to provide those of ordinary skill in the art with a complete disclosure and description of how to make and use the subject invention, and are not intended to limit the scope of what is regarded as the invention.

### Experimental

Progenitor identification and SARS-CoV-2 infection in long-term human distal lung organoid cultures

The distal lung contains terminal bronchioles and alveoli that facilitate gas exchange and is affected by disorders including interstitial lung disease, cancer, and SARS-CoV-2-associated COVID-19 pneumonia. Investigations of these localized pathologies have been hindered by a lack of 3D in vitro human distal lung culture systems. Further, human distal lung stem cell identification has been impaired by quiescence, anatomic divergence from mouse and lack of lineage tracing and clonogenic culture.

Here, we developed robust feeder-free, chemically-defined culture of distal human lung progenitors as organoids derived clonally from single adult human alveolar epithelial type II (AT2) or KRT5+ basal cells. AT2 organoids exhibited AT1 transdifferentiation potential, while basal cell organoids progressively developed lumens lined by differentiated club and ciliated cells. Organoids consisting solely of club cells were not observed. Upon single cell RNA-sequencing (scRNA-seq), alveolar organoids were composed of proliferative AT2 cells; however, basal organoid KRT5+ cells contained a distinct ITGA6+ITGB4+ mitotic population whose proliferation segregated to a TNFRSF12Ah′ subfraction.

Clonogenic organoid growth was markedly enriched within the TNFRSF12A^{hi} subset of FACS-purified ITGA6+ITGB4+ basal cells from human lung or derivative organoids. In vivo, TNFRSF12A+ cells comprised -10% of KRT5+ basal cells and resided in clusters within terminal bronchioles.

To model COVID-19 distal lung disease, we everted the polarity of basal and alveolar organoids to rapidly relocate differentiated club and ciliated cells from the organoid lumen to the exterior surface, thus displaying the SARS-CoV-2 receptor ACE2 on the outwardly-facing apical aspect. Accordingly, basal and AT2 “apical-out” organoids were infected by SARS-CoV-2, identifying club cells as a novel target population. This long-term, feeder-free organoid culture of human distal lung alveolar and basal stem cells, coupled with single cell analysis, identifies unsuspected basal cell functional heterogeneity and exemplifies progenitor identification within a slowly proliferating human tissue. Our studies establish a facile in vitro organoid model for human distal lung infectious diseases including COVID-19-associated pneumonia.

The distal lung, including terminal bronchioles and alveoli, performs essential gas exchange functions which can be significantly compromised by disease. For example, SARS-CoV-2 infection can elicit severe distal lung COVID-19 pathology with life-threatening pneumonia and respiratory failure. The limited understanding of COVID-19 pneumonia pathogenesis during a worldwide pandemic has highlighted a pressing need for robust in vitro culture systems allowing study of distal lung pathologies in primary human cells.

The lack of long-term human distal lung culture systems has precluded functional testing of the proliferative capacity of putative human distal lung stem cell populations, which are therefore largely inferred from mouse studies. In mouse, lineage-tracing has enabled in vivo confirmation and mapping of ‘bifunctional’ distal lung stem cells that constitutively execute both physiologic and regenerative functions, namely secretory club cells in distal bronchioles and surfactant-producing alveolar epithelial type II (AT2) cells in alveoli. Injury-inducible murine lung populations include an alveolar progenitor that renews AT1 and AT2 cells, and distal airway basal cell-like or bronchioalveolar progenitors with airway and alveolar differentiation potential. Whether the human correlates of these mouse stem cells are functional in renewing mature lung cell lineages is largely unknown.

The cell type composition of human terminal airways differs substantially from mouse. In the human lung basal cells span the entire airway axis, while in mouse they are absent from the terminal bronchioles where club cells renew and repair the epithelium. Long-term culture of human tracheal and bronchial basal cells have demonstrated stem cell potential, and these are also presumed to function as stem cells for lower airway renewal. Human AT2 cell cultures have also been reported but are short-lived, and the long-term self-renewal capacity of human AT2 cells thus remains unknown. Furthermore, existing AT2 cell culture protocols achieve only minimal expansion and require feeder cells producing unknown factors, limiting their biological characterization and screening utility. Directed differentiation of induced pluripotent stem cells (iPSCs) to AT2 cells can be limited by efficiency, feeder dependence and persistent fetal gene expression, suggesting immaturity. Here, we established long-term, feeder-free, chemically-defined 3D organoid culture of distal human lung, including AT2 and basal stem cells, and applied this method to progenitor identification and SARS-CoV-2 modeling.

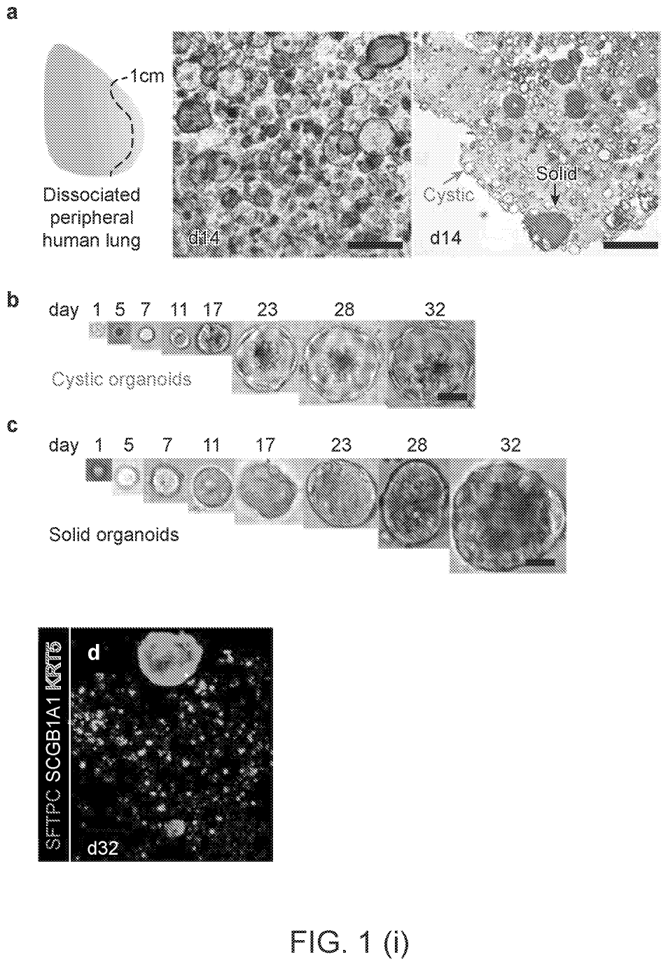

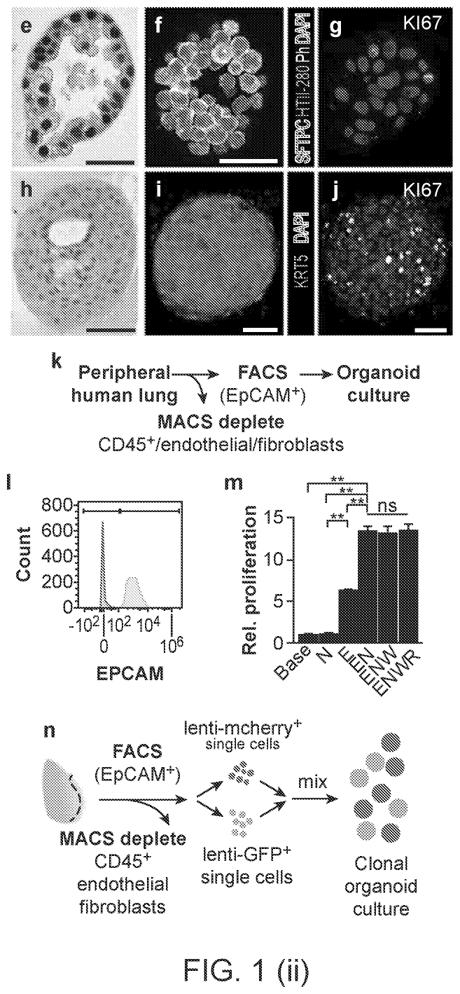

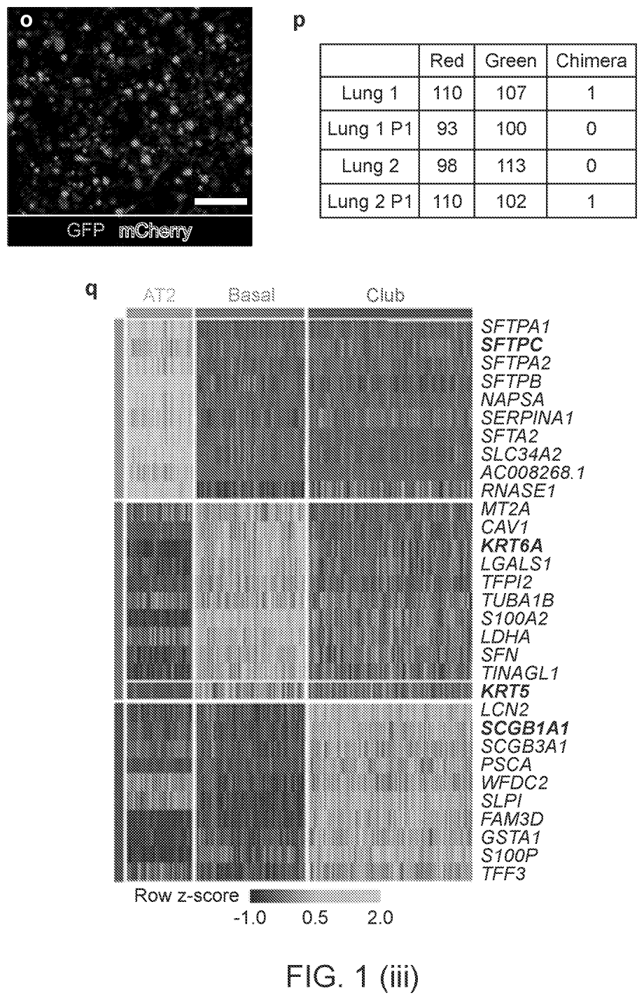

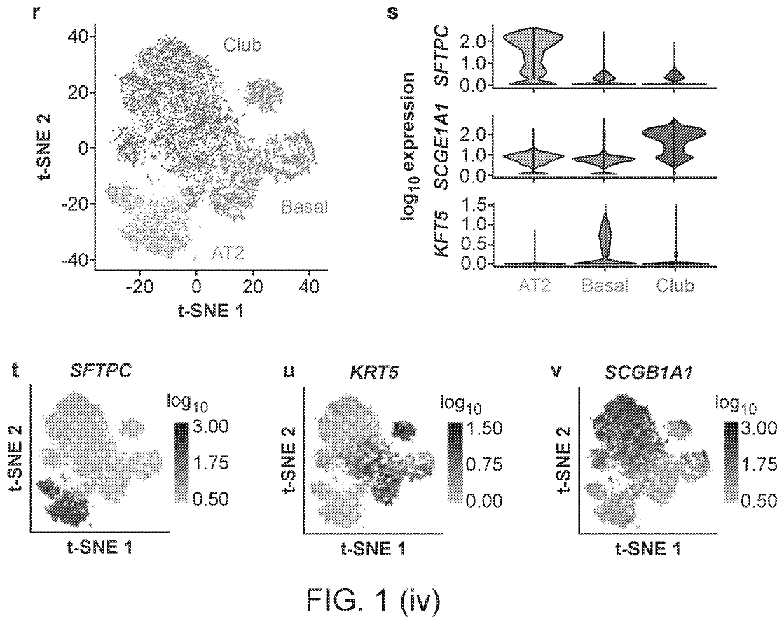

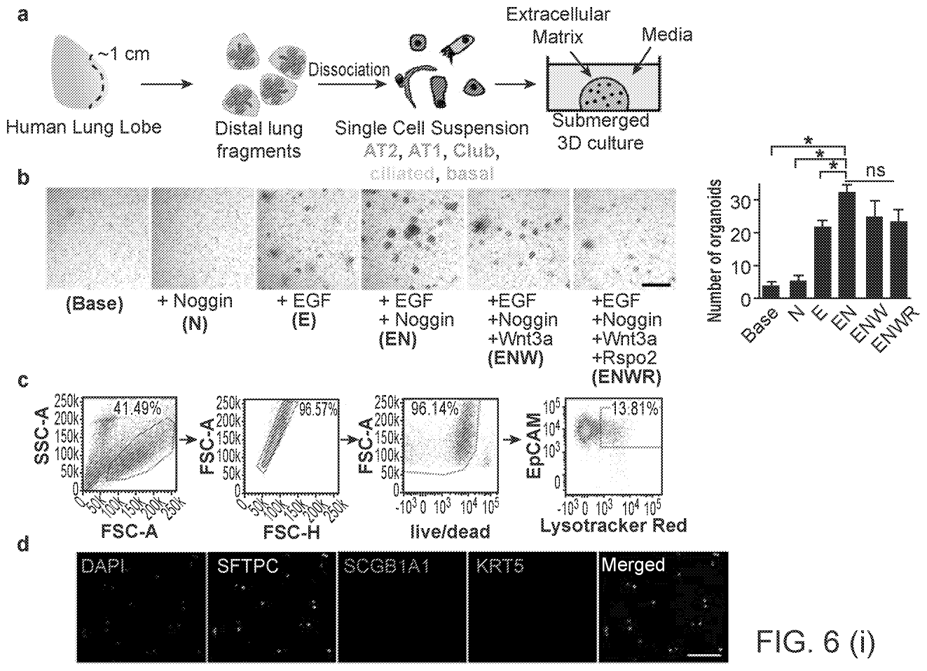

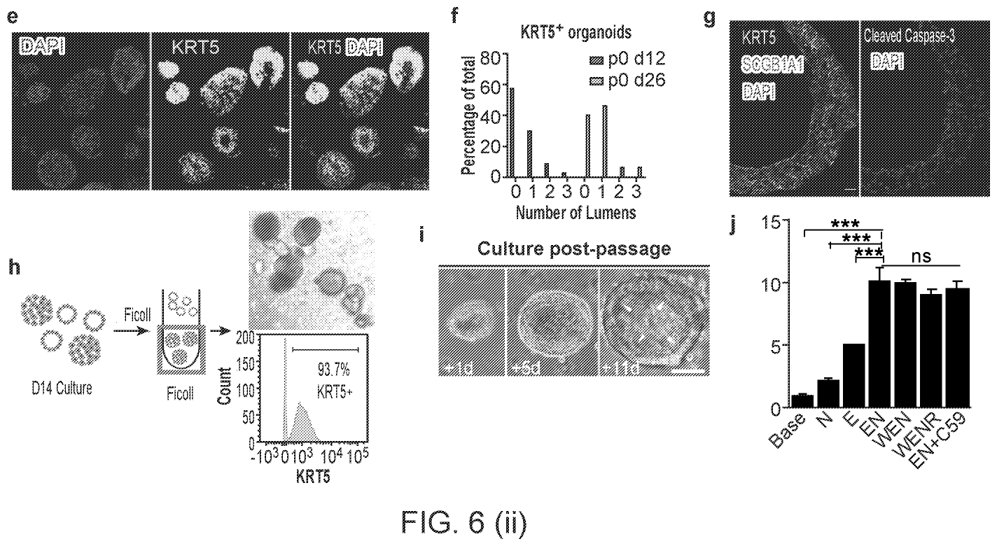

Human adult distal lung culture yields clonogenic alveolar and basal cell organoids. We empirically established defined media conditions supporting clonal expansion of distal human lung progenitors, encompassing bronchiolar and alveolar cells. To exclude proximal cell types, we used only the peripheral one centimeter of lung underlying the mesothelium, which was devoid of cartilage (FIG. 1 a). Single cell suspensions from 134 individuals (Table 1), were cultured within a droplet of collagen/laminin extracellular matrix without exogenous feeder cells. We surveyed growth factors whose cognate pathways including WNT, EGF and BMP have been implicated in lung development and disease pathogenesis. The combination of EGF and the BMP antagonist NOGGIN was optimal, without any additional growth-promoting effects of either WNT3A or R-SPONDIN1 (RSPO1) (FIG. 6a,b). Single cells underwent clonal expansion into one of two distinct organoid morphologies. Cystic organoids (FIG. 1a,b) were SFTPC⁺HT2-280⁺ and lacked KRT5, indicating AT2 cell identity (FIG. 1d-g). In contrast, solid organoids (FIG. 1a,c) expressed the basal cell marker KRT5 and lacked SFPTC indicating basal cell identity (FIG. 1d, h-j). Organoids consisting solely of SCGB1A1+ club cells were not observed (FIG. 1d).

To stringently exclude the possibility that contaminating stromal cells could be contributing unknown growth factors, we generated distal lung organoids from >99.9% EPCAM⁺ starting populations by magnetic bead depletion of fibroblasts, endothelial and hematopoietic cells followed by FACS purification of EPCAM⁺ cells, which confirmed organoid generation with only EGF and NOGGIN provision (FIG. 1 k-m). Distal lung organoids could be passaged for -6 months with basal organoids initially exhibiting 6-7 doublings every 2 weeks. Alveolar organoids expanded more slowly with an initial rate of 3-4 doublings/2 weeks but predominated over basal organoids after several months. Based on initial cell division rates , the upper limits of basal and alveolar expansion were 2¹⁹ (524,288 fold) and 2¹⁶ (65,536 fold) respectively. Organoids arose clonally as confirmed by (1) time-lapse microscopy of single cells (FIG. 1b,c) and (2) color mixing studies of disaggregated, lentivirally-transduced GFP⁺ or mCherry⁺ cells that generated entirely red or green but not chimeric organoids (FIG. 1n-p).

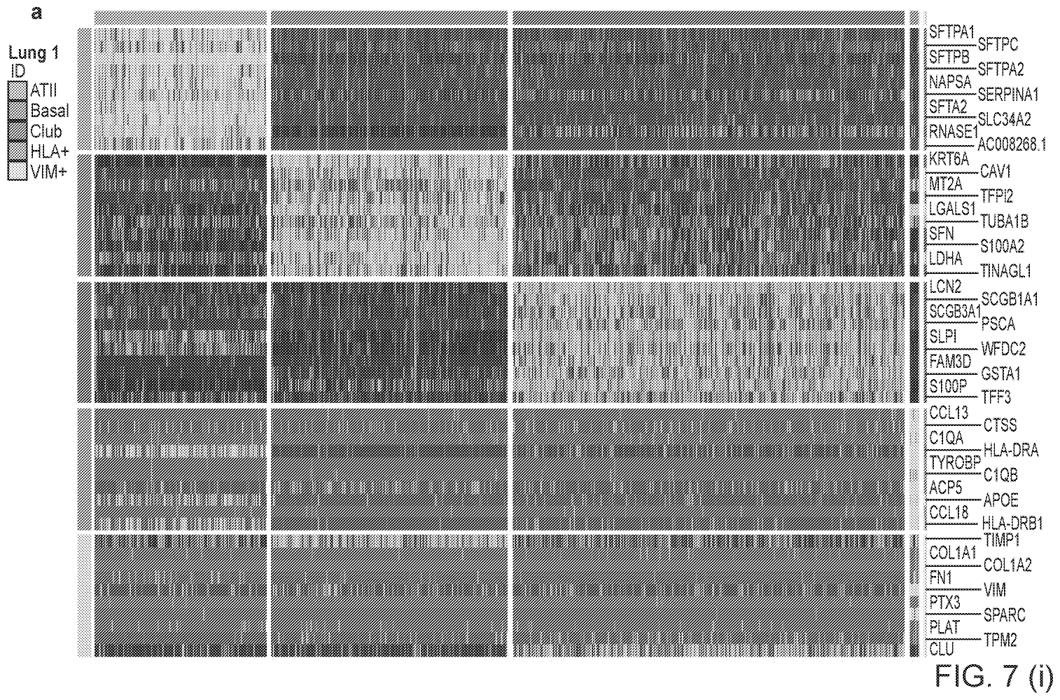

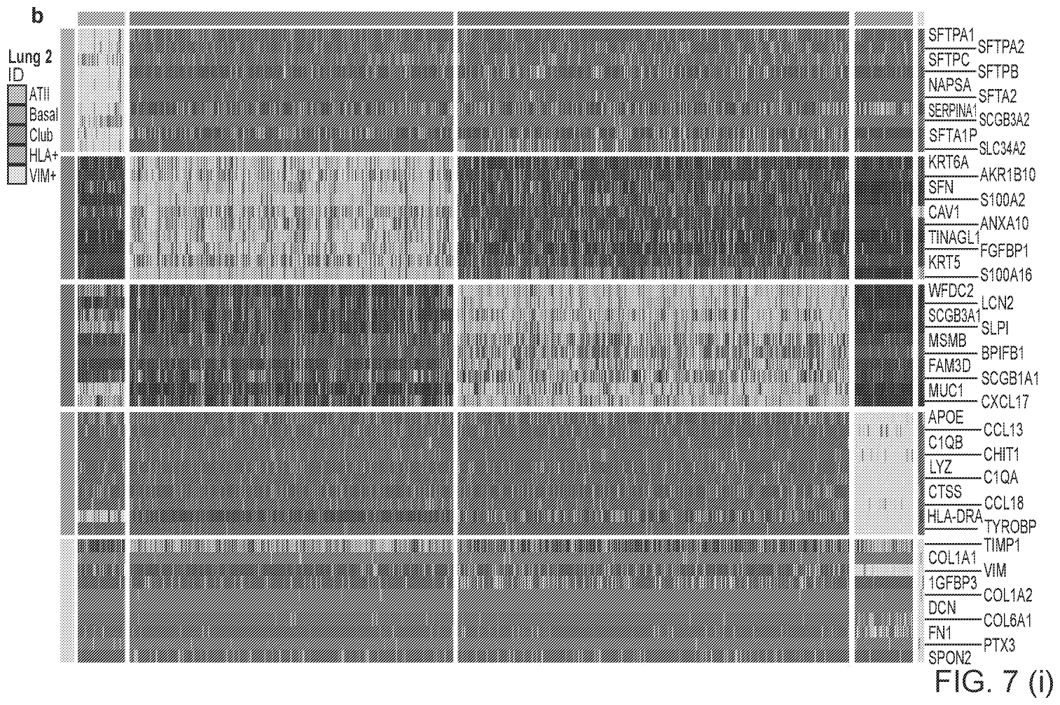

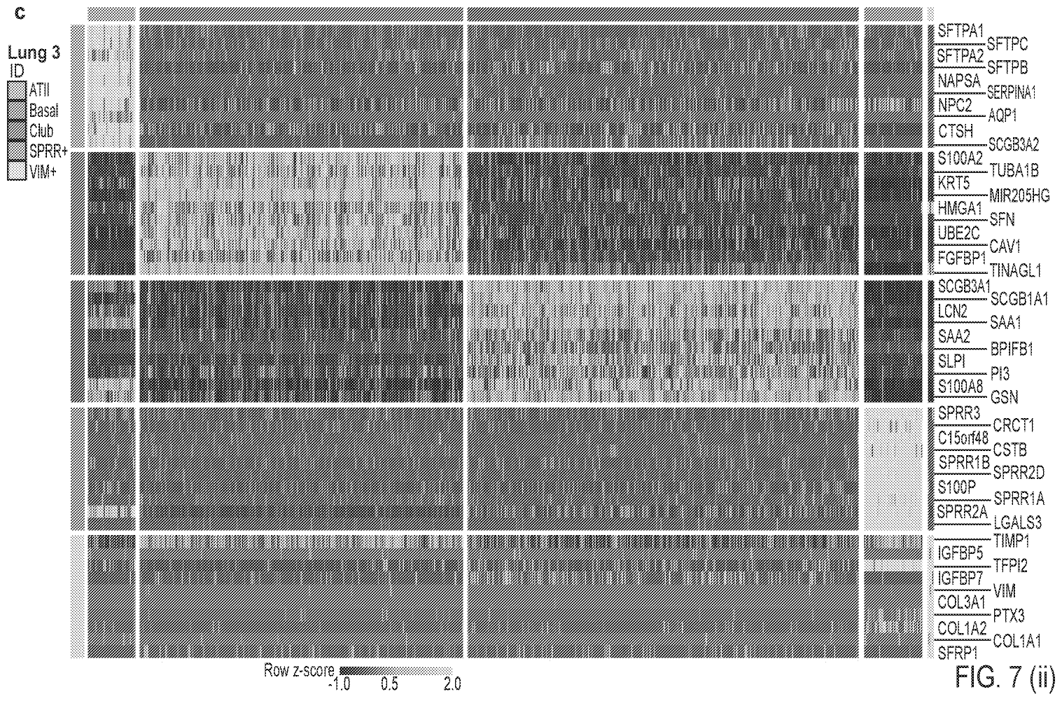

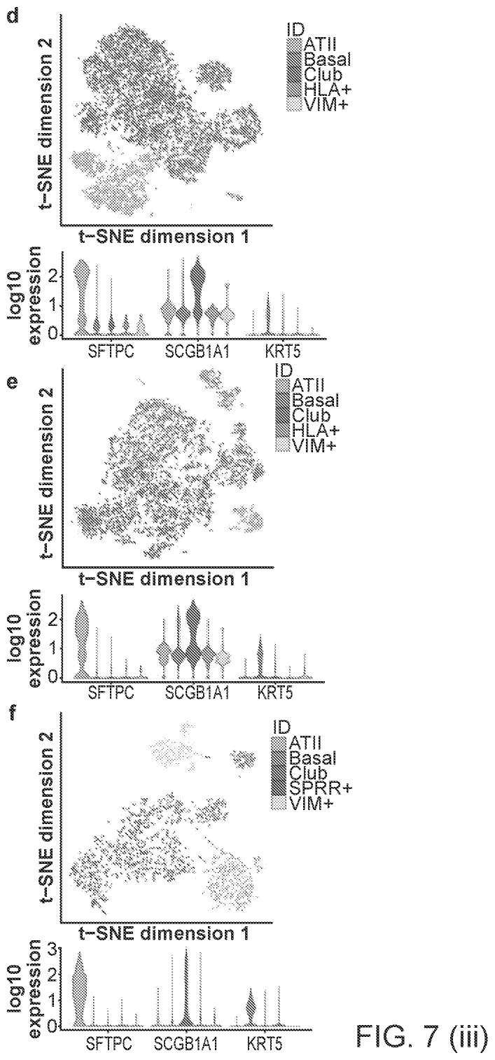

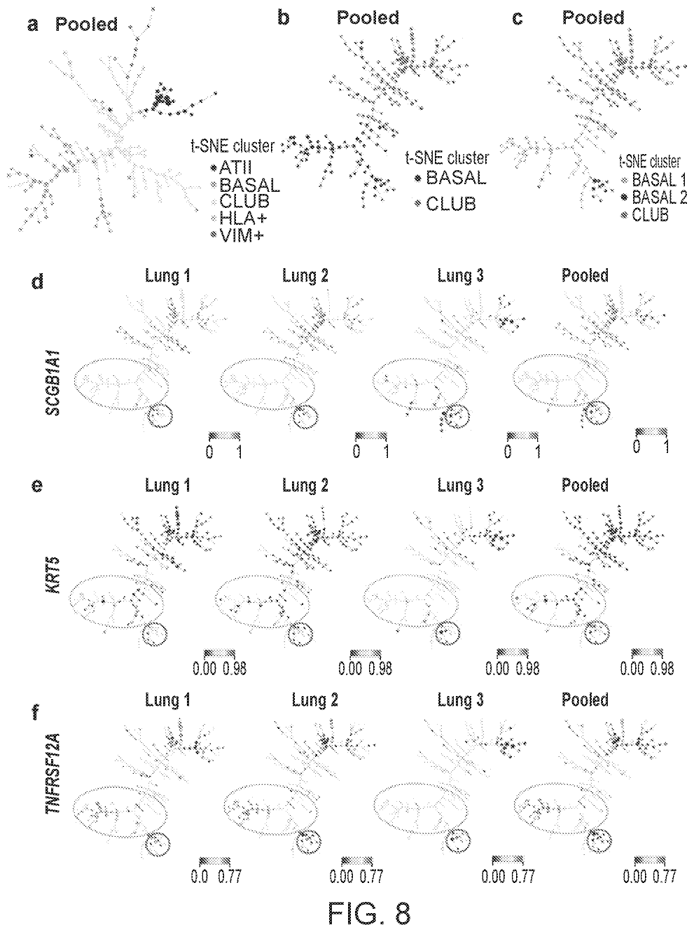

Single cell RNA-sequencing of distal lung organoids confirmed distinct SFTPC+ AT2, KRT5⁺ basal and SCGB1A1⁺ club cell populations. Cells co-expressing KRT5 and SCGB1A1 bridged the basal and club cell populations, suggesting molecular intermediates transitioning from basal into club cells (FIG. 1q-v, FIG. 7). Trajectory analysis using SPADE projected a cellular differentiation path from basal to club cell identity but not between AT2 and club or AT2 and basal cells (FIG. 8).

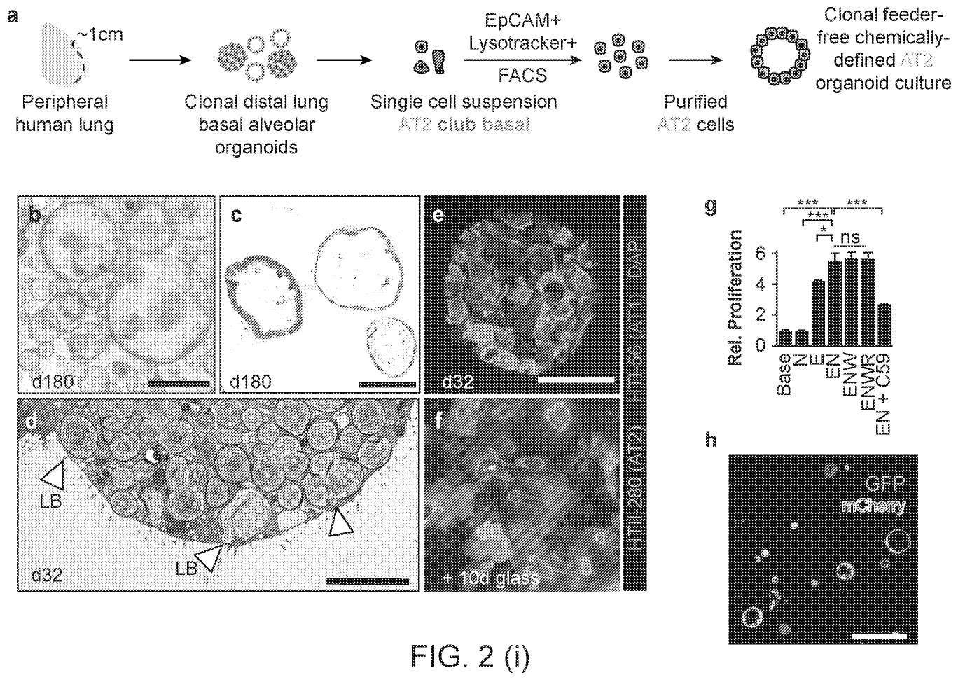

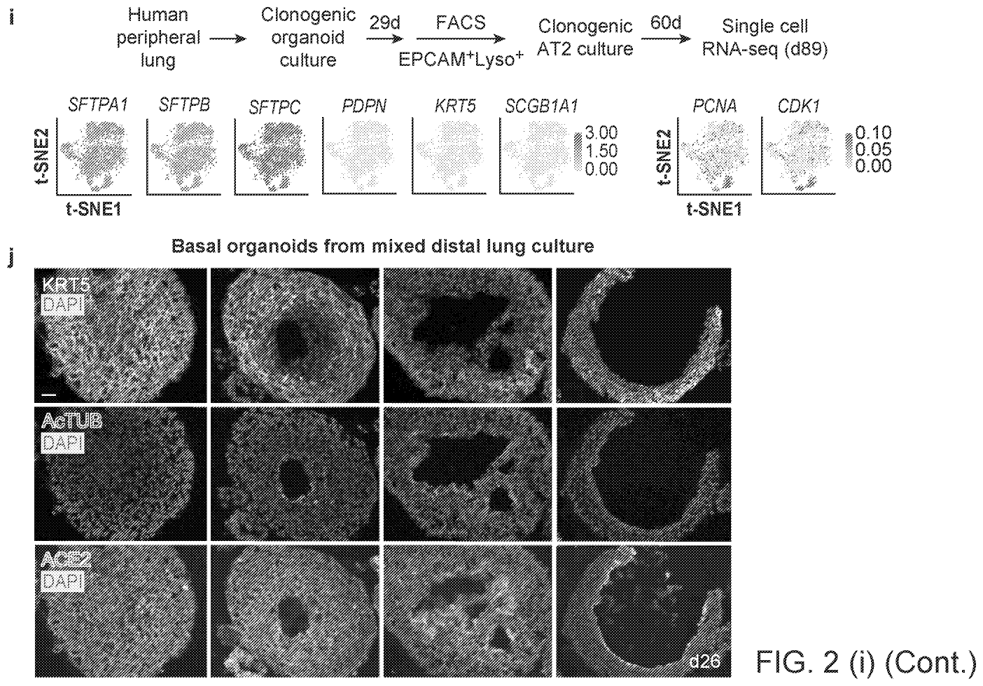

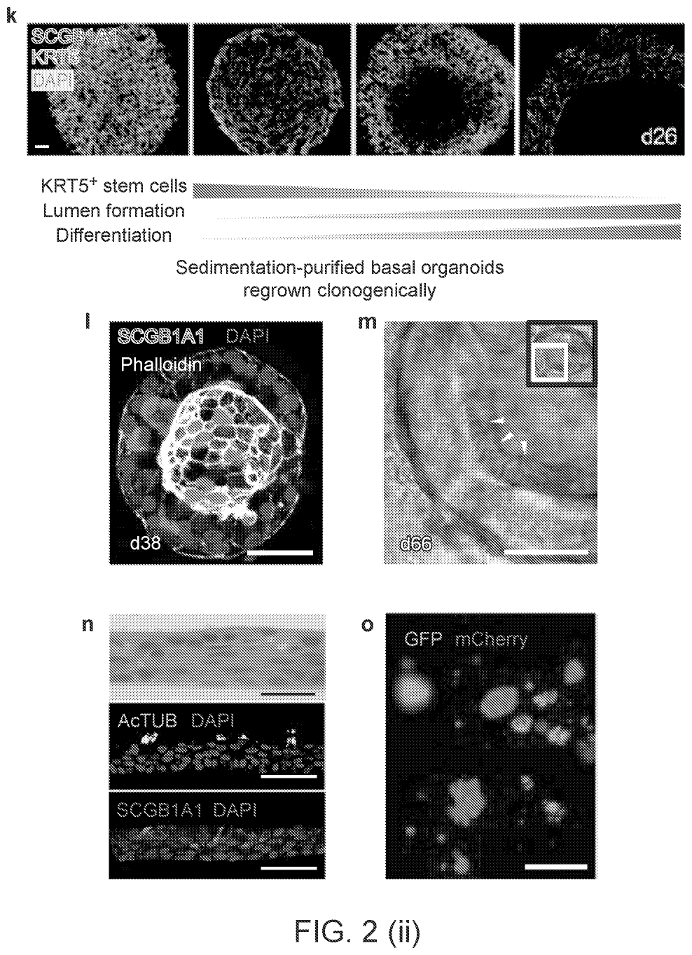

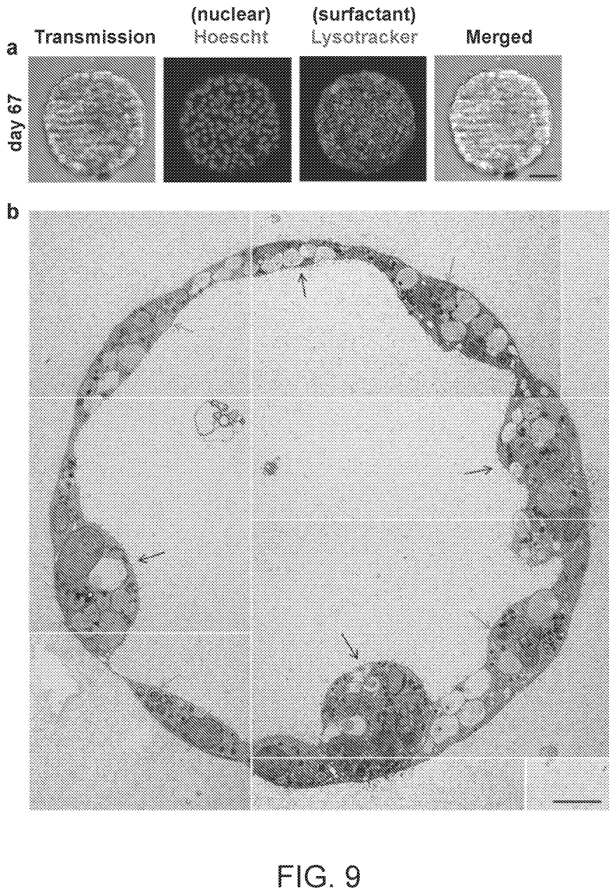

Human AT2 cells extensively renew and maintain AT1 cell transdifferentiation capacity. We further refined these methods to generate pure clonogenically-derived AT2 organoids. Viable AT2 cells were purified from mixed distal lung organoids without accompanying stromal populations using fluorescence-associated cell sorting (FACS), exploiting lamellar body uptake of the lysosomal dye LysoTracker in EPCAM⁺ cells (FIG. 2a, FIG. 6c-d, FIG. 9a). Individual EPCAM⁺LysoTracker⁺ AT2 cells progressively expanded as cystic organoids up to 180 days, exhibiting a mixture of cuboidal or more flattened morphologies reminiscent of alveoli (FIG. 2b,c). Qualitatively identical results were obtained with anti-HTII-280 (AT2 marker) purification instead of LysoTracker. Transmission electron microscopy (TEM) revealed a basal surface contacting surrounding matrix, characteristic apical microvilli, and abundant cytoplasmic lamellar bodies, characteristic of fully mature and functional AT2 cells (FIG. 2d, FIG. 9b). AT2 organoids expressed HTII-280 (FIG. 2e) and occasionally assumed an AT1-like flattened morphology with variable downregulation of AT2 cell type markers but without AT1 marker expression (data not shown). However, upon culture on a glass surface with fetal bovine serum which promotes the AT1 phenotype, AT2 cells rapidly flattened, downregulated HTII-280 and initiated AT1 HTI-56 marker expression, indicating retention of differentiation capacity after expansion (FIG. 2f). EGF and NOGGIN were sufficient for clonal AT2 organoid proliferation and exogenous WNT-3A and RSPONDIN-1 did not enhance growth. However, the PORCUPINE inhibitor C59, which blocks endogenous WNT biosynthesis, attenuated AT2 organoid growth (FIG. 2g), suggesting essential autocrine WNT signaling and recapitulating mouse AT2 cell biology. Lastly, isolated AT2 cells exhibited clonal organoid growth in lentivirus GFP/mCherry mixing studies, with 0/797 organoids demonstrating chimerism (FIG. 2h).

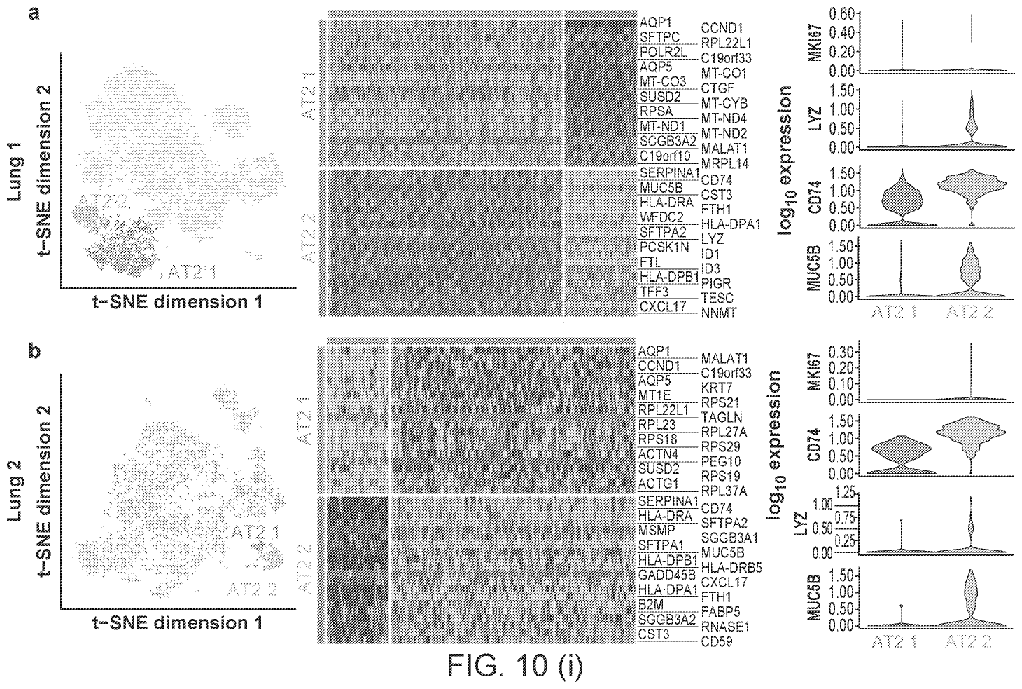

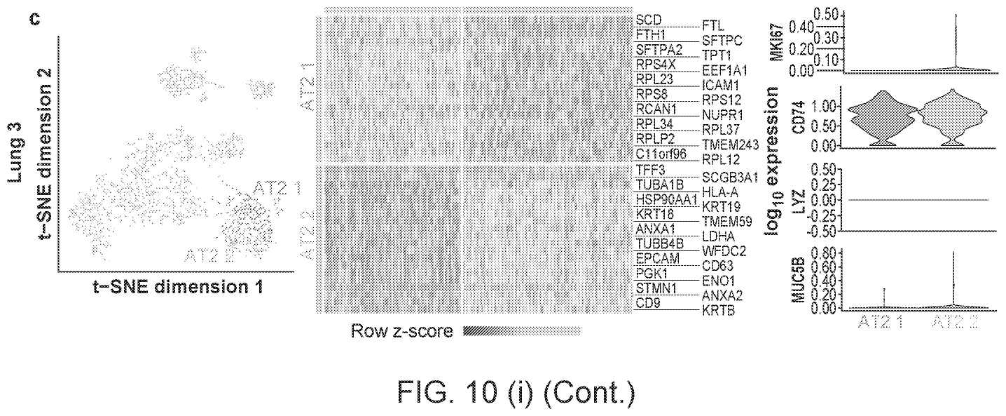

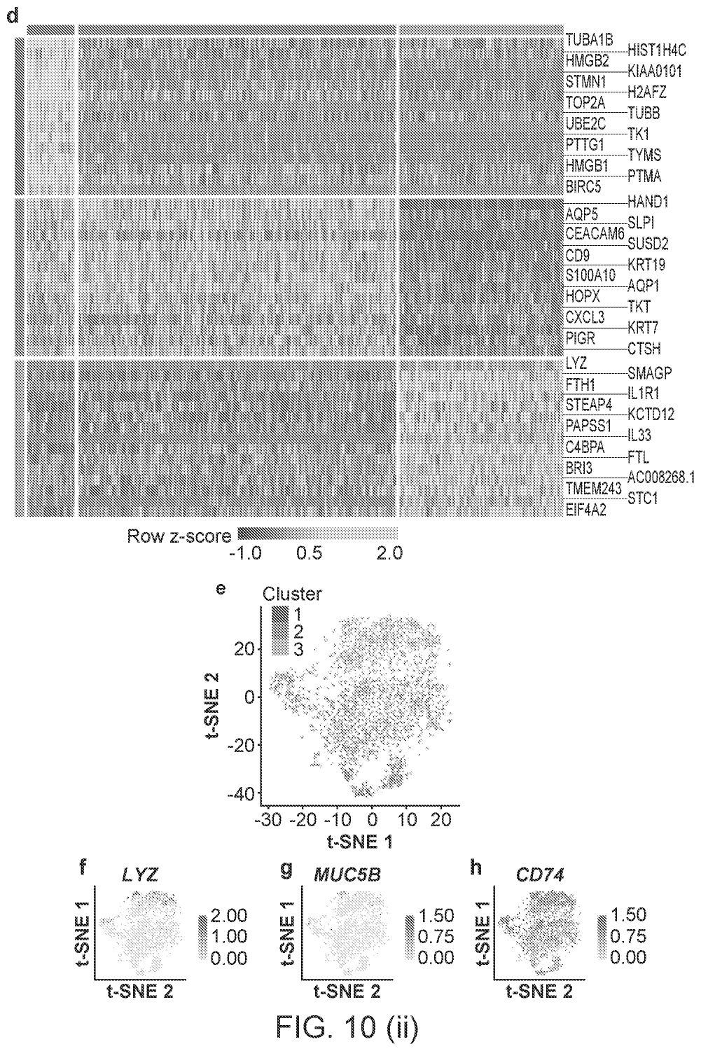

Single cell RNA-seq of mixed distal lung organoids revealed uniformly high-level expression of canonical AT2 cell markers such as SFTPC within the alveolar populations (FIG. 1q-t, FIG. 7). These data did not readily identify AT2 cell subsets within organoids, although the relatively low number of AT2 cells limited sensitivity (FIG. 7, FIG. 10a-c). We thus generated clonally derived pure alveolar organoids by culturing FACS-isolated EPCAM⁺Lysotracker⁺ cells from mixed distal lung organoids (FIG. 2i). Analysis of 2,780 single cells from purified alveolar organoids at culture day 89 (60 days post-FACS purification) re-demonstrated homogeneous AT2 marker expression including SFTPA1, SFTPB and SFTPC without basal, club, or AT1 markers (FIG. 2i). Importantly, cell cycle mRNAs such as PCNA and CDK1 were expressed by a minority of AT2 cells but did not cluster to a specific population (FIG. 2i) and a proliferative sub-cluster within AT2 organoids having distinct gene expression unrelated to cell cycle status was not detected (FIG. 10d-h).

Spontaneous differentiation of human distal airway basal cell-derived organoids. Basal cell organoids in mixed distal lung culture initially formed solid KRT5+ masses (FIG. 1a, c-d). However, by ˜1 month, approximately 50% stochastically developed single but occasionally multiple lumens; this lumen formation did not result from apoptosis (FIG. 2j, FIG. 6e-g). Lumen appearance coincided strongly with the emergence of differentiated acetylated tubulin+(AcTUB⁺) ciliated cells and SCGB1A⁺ club cells at the lumenal surface. Conversely, the basal stem cell marker KRT5 was specifically excluded from the differentiated lumen zone, but was otherwise diffusely expressed (FIG. 2j,k).

We further established pure basal cell cultures by using density sedimentation to remove cystic AT2 organoids, leaving behind solid basal organoids which were then disaggregated and regrown from single cell suspensions. After 2-4 weeks in culture post-sedimentation, proliferating basal cell organoids again progressively cavitated (FIG. 6h-i) with appearance of luminal SCGB1A1⁺ club and AcTUB⁺ ciliated cells either upon organoid culture (FIG. 2l, m) or conversion of 3D organoids to 2D air-liquid interface monolayers (FIG. 2n). Clonal outgrowth of basal cell-derived organoids was confirmed by density sedimentation followed by FACS isolation and culture of EPCAM⁺ cells and culture which exhibited monoclonality in lentivirus GFP/mCherry mixing studies with only 2/845 chimeric organoids (FIG. 2o). Under these conditions EGF and Noggin were again sufficient for maximal growth without additive effects of WNT3A, R-SPONDIN1. Unlike AT2 organoids, growth was not significantly inhibited by the PORCUPINE inhibitor C59 (FIG. 6j).

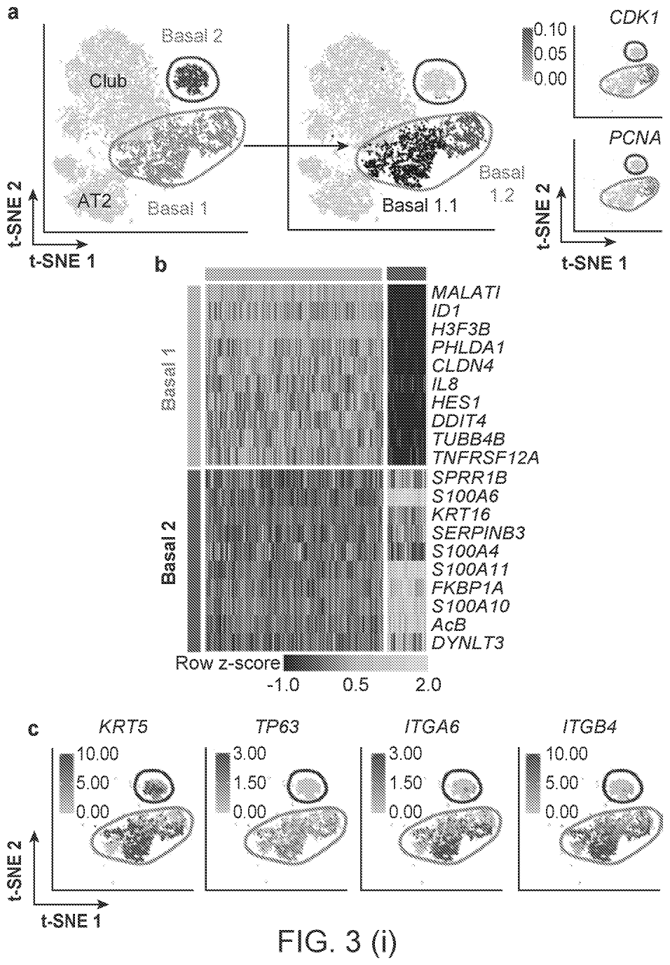

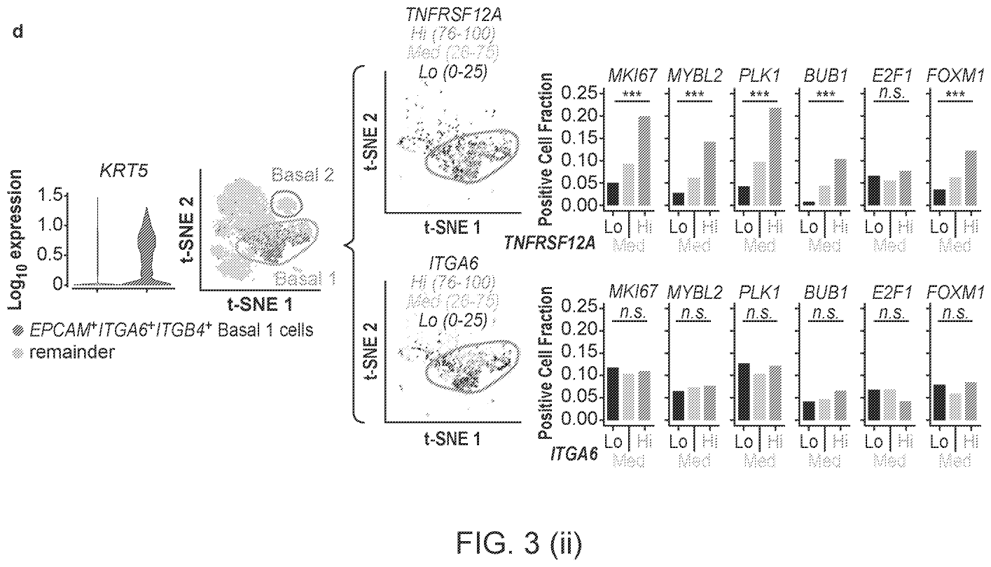

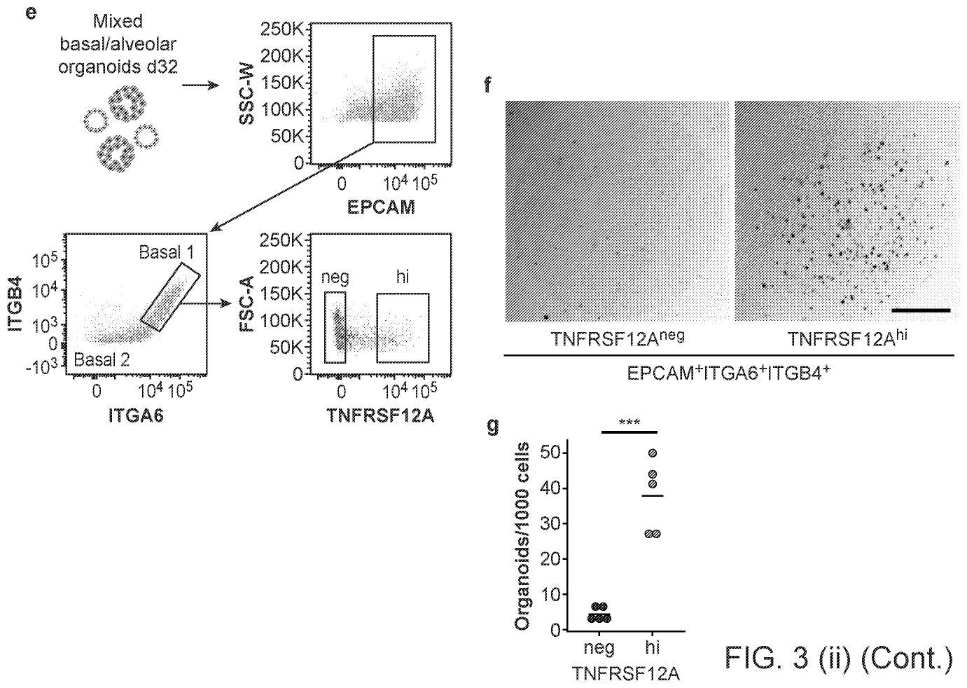

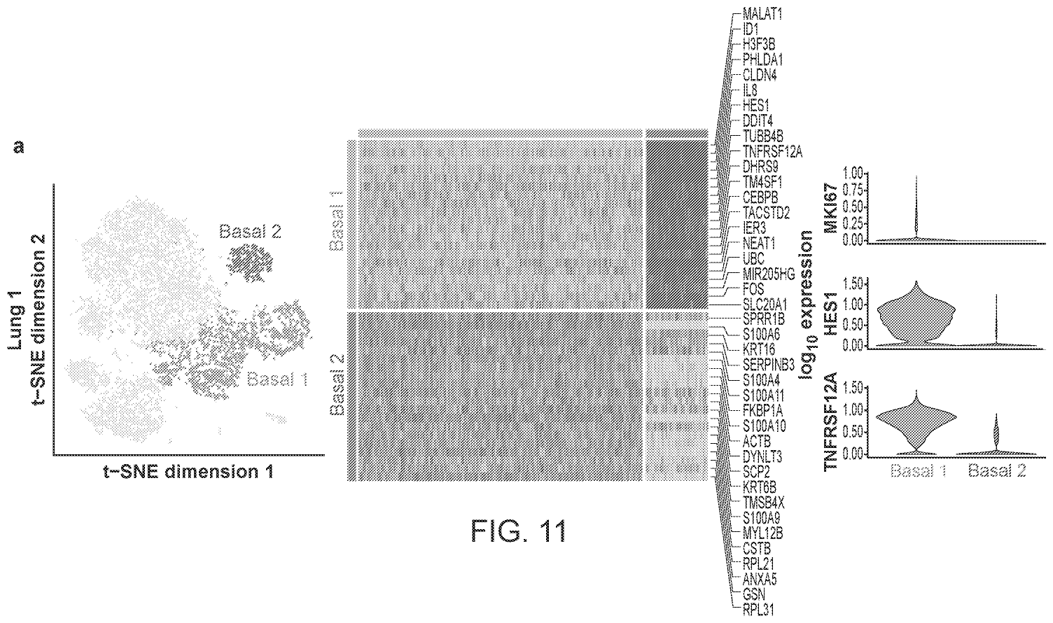

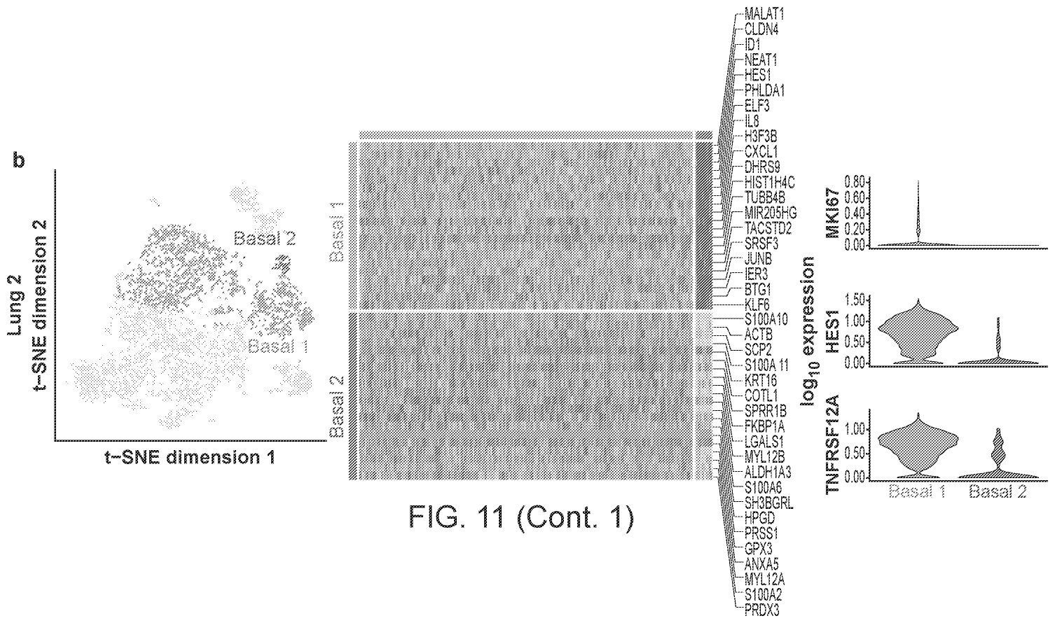

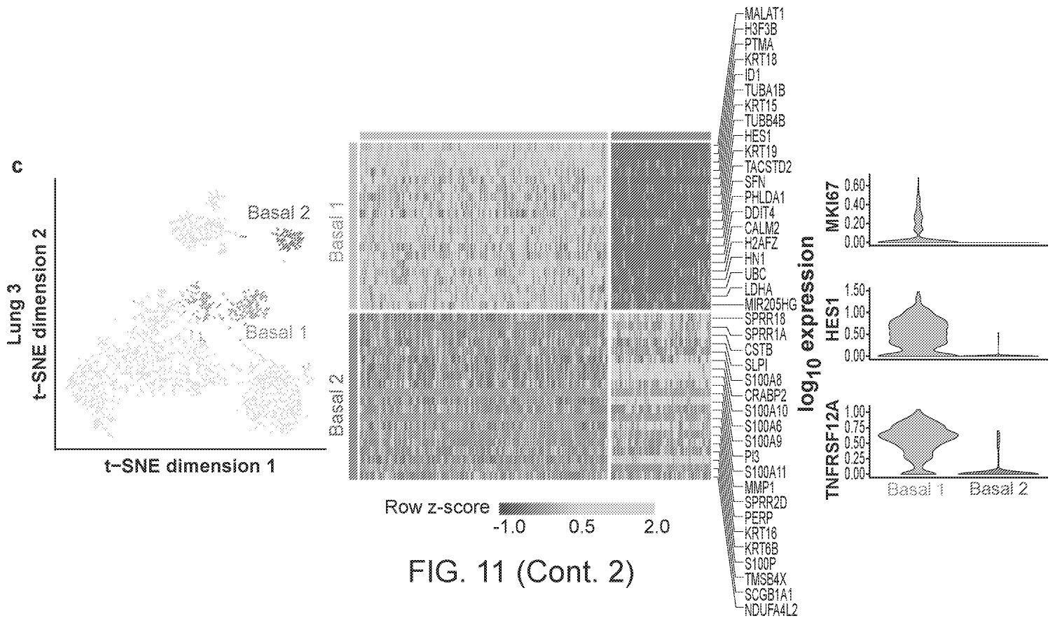

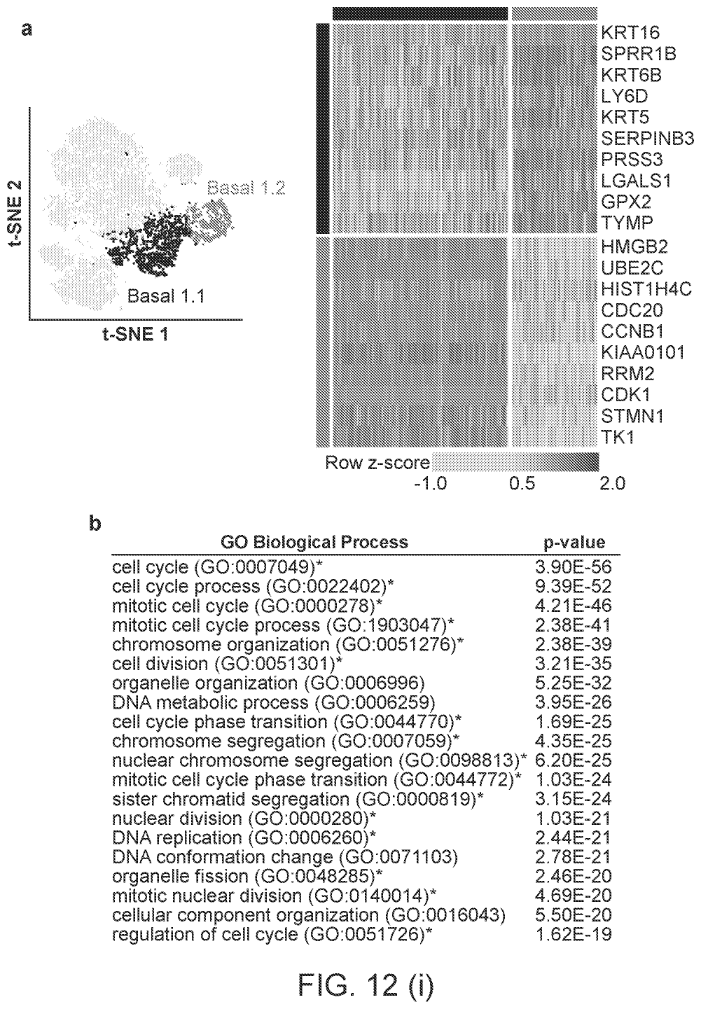

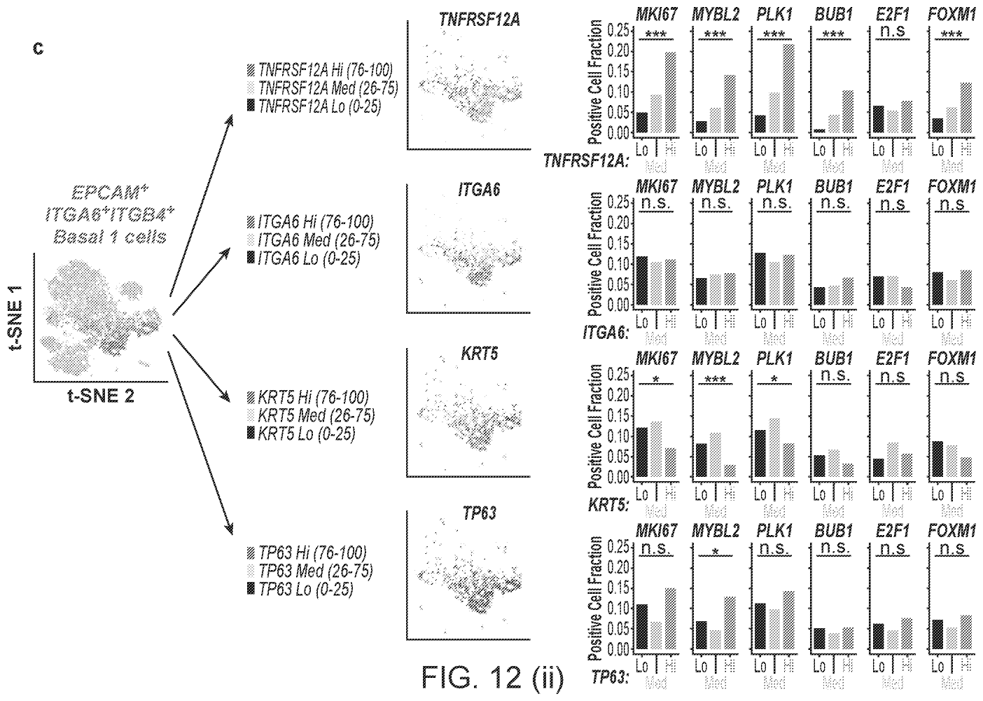

Organoid scRNA-seq reveals two molecularly distinct subtypes of human distal airway basal cells. In contrast to the homogeneity of organoid AT2 cells, scRNA-seq clustering of KRT5⁺ basal cells from multiple individuals reproducibly identified two subpopulations, designated Basal 1 and Basal 2 (FIG. 3a-b, FIG. 11). Basal 1 was enriched for differentiation and cell fate determinants such as HES1 and ID1 and included an actively cycling subpopulation expressing proliferation markers PCNA and CDK1 with significantly overrepresented GSEA cell cycle processes (FIG. 3a, FIG. 12a-b). Basal 1 but not Basal 2 included canonical lung basal cell mRNAs such as integrin α₆ (ITGA6) and TP63, as well as integrin β₄ (ITGB4) which is a binding partner for integrin α₆ and also expressed in murine Lineage Negative Epithelial Progenitors (LNEPs) (FIG. 3c). Basal 2, while lacking the above Basal 1 proliferative and cell fate markers, was enriched in vesicular transport, endoplasmic reticulum housekeeping processes and squamous markers; these transcripts were also present in Basal 1, albeit at lower levels.

TNFRSF12A marks an organoid basal cell subpopulation with enriched progenitor activity. We next examined the membrane receptor TNFRSF12A (Fn14, TweakR), one of the most differentially expressed genes in the Basal 1 cluster (FIG. 3b, Table 2), because of its potential utility for FACS sorting and since a related TNF superfamily membrane receptor gene family member, TNFRSF19, marks gastric and intestinal stem cells. Indeed, TNFRSF12A mRNA was enriched in EPCAM³⁰ ITGA6³⁰ ITGB4³⁰ Basal 1 versus Basal 2 cells (FIG. 3d). In scRNA-seq, Basal 1 EPCAM³⁰ ITGA6³⁰ ITGB4³⁰ cells could be divided into TNFRSF12A-low, -medium and -high mRNA-expressing fractions, and notably a proliferative gene module was significantly enriched in the highest (TNFRSF12A^{hi}) versus lowest quartile (TNFRSF12A^{lo}) subsets (FIG. 3d). Crucially, low, medium and high expression of the Basal 1 transcripts ITGA6 or TP63, or KRT5 did not enrich for this proliferative signature, indicating particular discriminatory utility of TNFRSF12A mRNA levels (FIG. 3d, FIG. 12c). To functionally validate this observation, we fractionated total distal lung organoids by anti-TNFRSF12A monoclonal antibody FACS into EPCAM³⁰ ITGA6³⁰ ITGB4³⁰ cells and then into TNFRSF12A^{hi} and TNFRSF12A^{neg} subsets. The incidence of TNFRSF12A^{hi} cells declined with time to comprise a minority of EPCAM³⁰ ITGA6³⁰ ITGB4³⁰ cells in late stage differentiating cultures (FIG. 3e). Notably, organoid-derived TNFRSF12A^{hi} basal cells reproducibly exhibited 4-12× greater clonogenic organoid-forming capacity than TNFRSF12A^{neg} cells in 5 out of 5 individuals (FIG. 3f,g) in parallel with >60-fold enrichment of TNFRSF12A mRNA in the former.

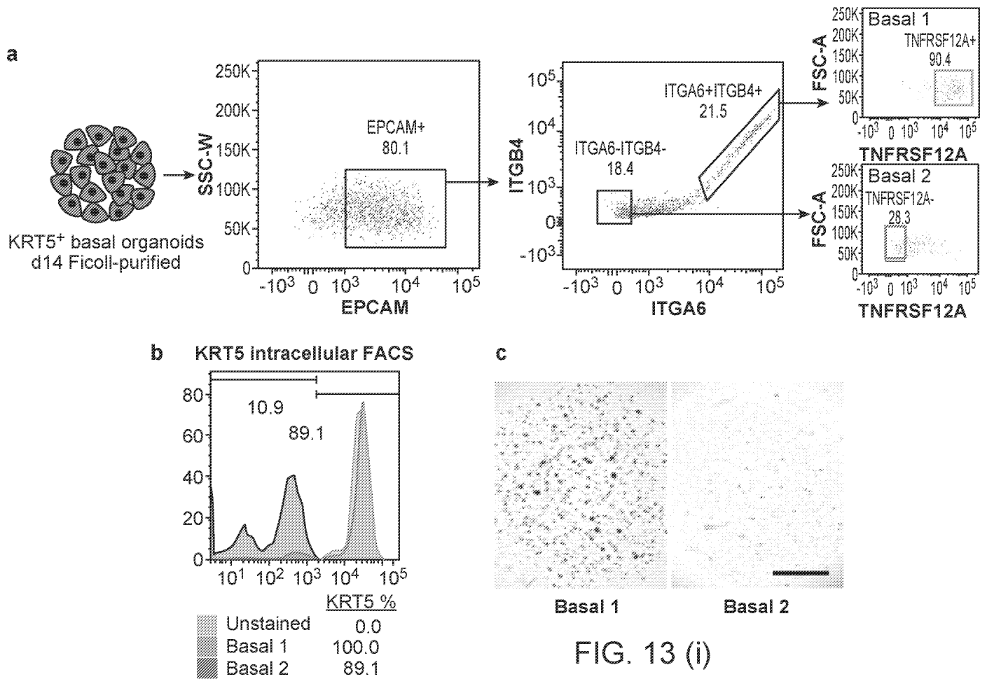

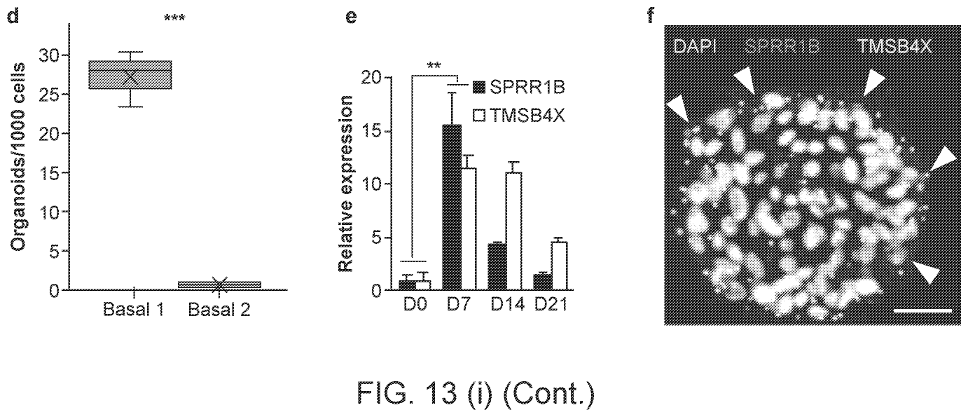

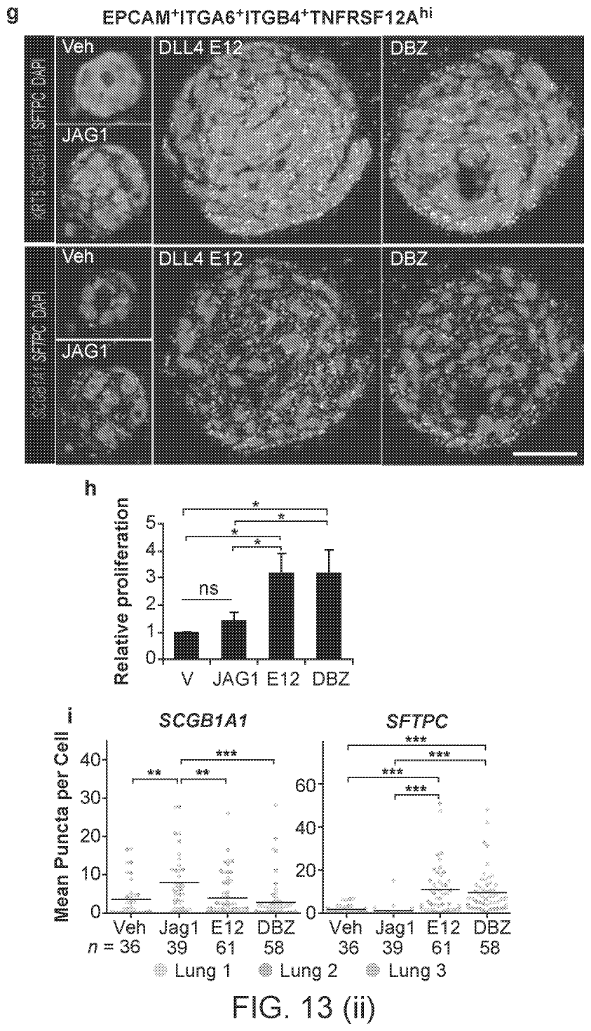

Possible lineage relationships between Basal 1 and Basal 2 were examined by FACS purification followed by clonogenic culture. Density sedimentation-purified KRT5+ basal organoids (FIG. 2l-n) were fractionated into EPCAM⁺ITGA6⁺ITGB4⁺TNFRSF12A^{hi} (Basal 1) and EPCAM⁺ITGA6⁻ITGB4⁻TNFRSF12A^{neg} (Basal 2) populations (FIG. 13a). Despite essentially homogeneous KRT5 expression in both fractions (FIG. 13b) clonogenic organoid formation was strongly enriched in Basal 1 versus Basal 2 from three separate individuals (FIG. 13c-d), indicating the lack of Basal 1 cell generation by Basal 2 cells. Basal 2-enriched loci SPRR1B and TMSB4X (FIG. 3b) were transiently induced in organoids from FACS-isolated TNFRSF12A^{hi} Basal 1 cells (FIG. 13e-f), indicating Basal 2 differentiation from Basal 1. SPADE trajectory analysis also demonstrated Basal 2 cells emanating from TNFRSF12A⁺ basal cells, independently supporting a one-way lineage route from Basal 1 to Basal 2 (FIG. 8).

The NOTCH target gene HES1 was one of the most differentially upregulated Basal 1 genes, and Basal 1-upregulated gene networks included NOTCH1, NOTCH2 and JAG1 (FIG. 3b). In basal organoids from FACS-purified TNFRSF12A^{hi}EPCAM⁺ITGA6⁺ITGB4⁺ cells, NOTCH inhibition by the gamma-secretase inhibitor DBZ or the extracellular domain of the DLL4 E12 mutant significantly increased proliferation (FIG. 13g-h), suggesting NOTCH restrains growth. NOTCH inhibition by DBZ or DLL4 E12 NOTCH signaling also stimulated incomplete alveolar TNFRSF12A^{hi} differentiation by upregulating SFTPC mRNA without lamellar body or SFTPC protein production (data not shown), mirroring the effect of Notch on mouse LNEP stem cells. Conversely, NOTCH agonism by JAG1 peptide did not affect proliferation but induced SCGB1A1, similar to reports in upper airway cells (FIG. 13i).

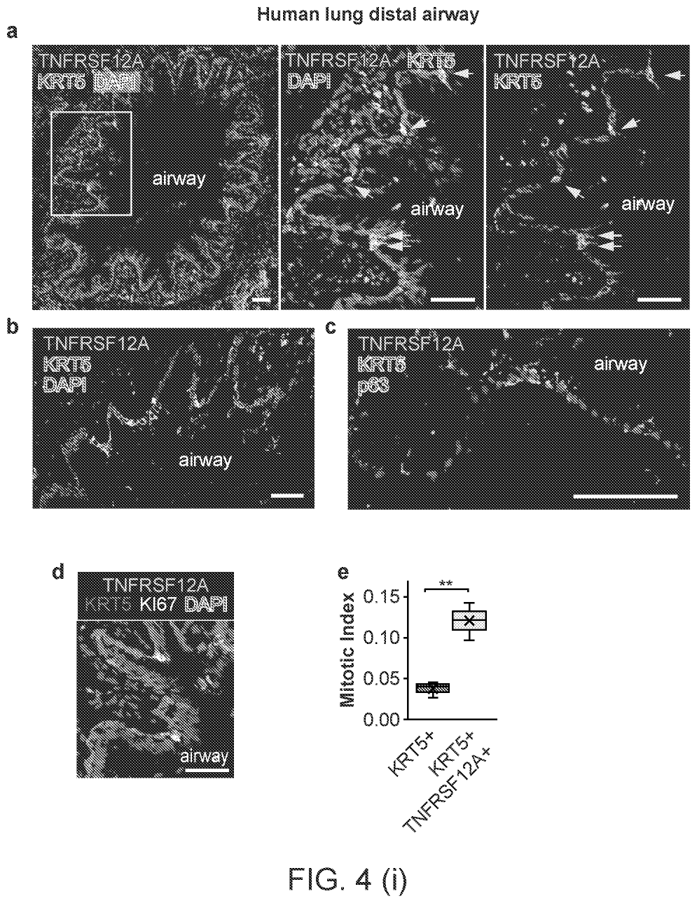

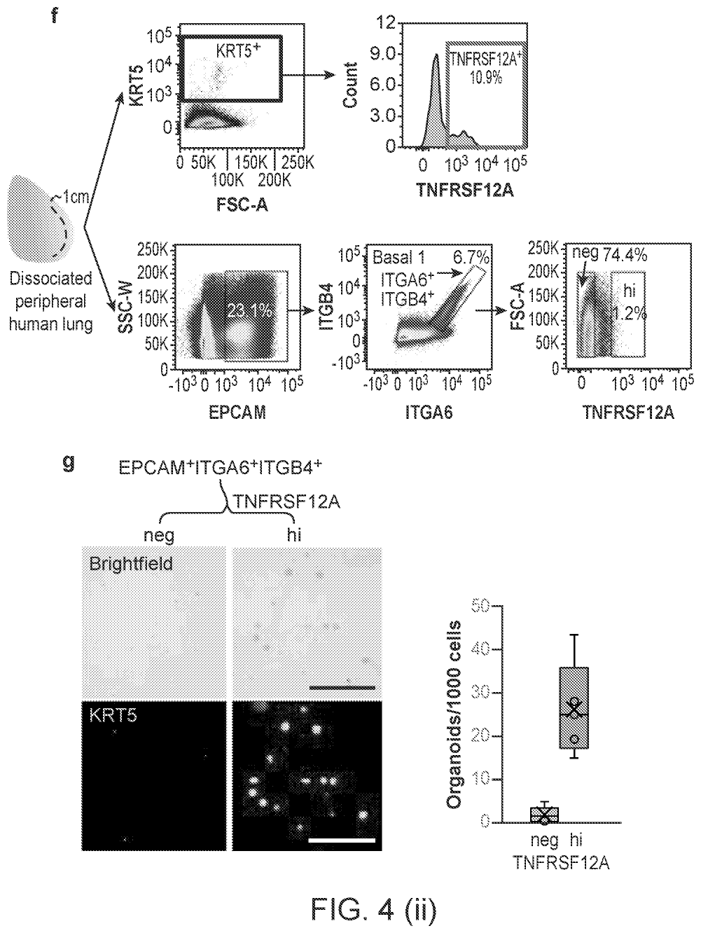

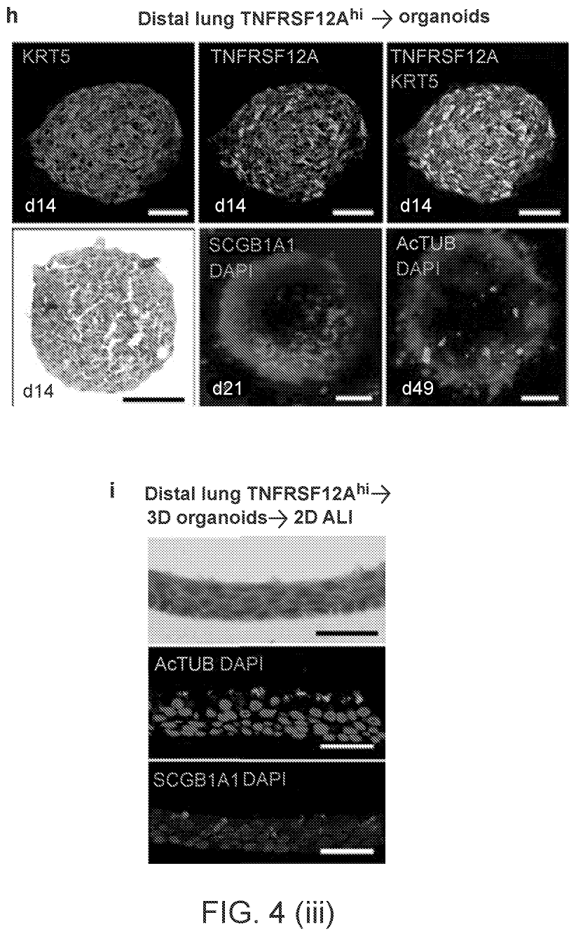

TNFRSF12A⁺ basal cells cluster within distal airways in vivo and exhibit enhanced clonogenic potential. To demonstrate the TNFRSF12A-expressing basal subpopulation in vivo we performed TNFRSF12A antibody staining of freshly fixed intact human distal lung specimens. KRT5 and/or p63 marked essentially all distal airway basal cells, but TNFRSF12A strikingly labeled a cell subset localized in sporadic clusters within the basal layer (FIG. 4a-c). KRT5+TNFRSF12A⁺ basal cells frequently, but not exclusively, resided at tips or bases of bronchiolar furrows, the latter a previously recognized niche for goblet cells. TNFRSF12A was not restricted to the basal layer and was detected in diverse lung stromal and epithelial cells, yet clearly marked a minor population of KRT5⁺/p63⁺basal cells (FIG. 4a-c). The TNFRSF12A-expressing subset of KRT5⁺ basal cells exhibited a higher mitotic index than total KRT5⁺ cells in vivo, consistent with enriched proliferative capacity in vitro (FIG. 4d-e). FACS analysis of freshly dissociated human lung confirmed TNFRSF12A expression in 10.9% of KRT5⁺ basal cells (FIG. 4f, top).

TNFRSF12A^{hi} Basal 1 cells could be prospectively isolated and cultured directly from freshly dissociated human lungs for culture without an organoid intermediate. EPCAM⁺ITGA6⁺ITGB4⁺TNFRSF12A^{hi} (i.e. TNFRSF12A^{hi} Basal 1) and EPCAM⁺ITGA6⁺ITGB4⁺TNFRSF12A^{neg} cells (i.e. TNFRSF12^{neg} Basal 1) were isolated from dissociated human lungs (FIG. 4f, bottom) and cultured clonogenically (FIG. 4g-i). Compared to TNFRSF12A^{neg} populations, TNFRSF12A^{hi} cells purified directly from distal lung exhibited a robust 15-fold increase in KRT5+ organoid formation (FIG. 4g), indicating substantial enrichment of organoid-generating capacity and recapitulating the enhanced clonogenic growth of organoid-derived TNFRSF12A^{hi} cells (FIG. 3f-g). Organoids grown from distal lung TNFRSF12A^{hi} cells also exhibited a characteristic basal histology and spontaneously differentiated to SCGB1A1⁺ club and ActTUB⁺ ciliated cells (FIG. 4h). Further, distal lung TNFRSF12A^{hi}-derived basal organoids adopted a typical stratified epithelial histology with apical ciliary and club cell differentiation upon re-plating as 2D air-liquid interface monolayers (FIG. 4i).

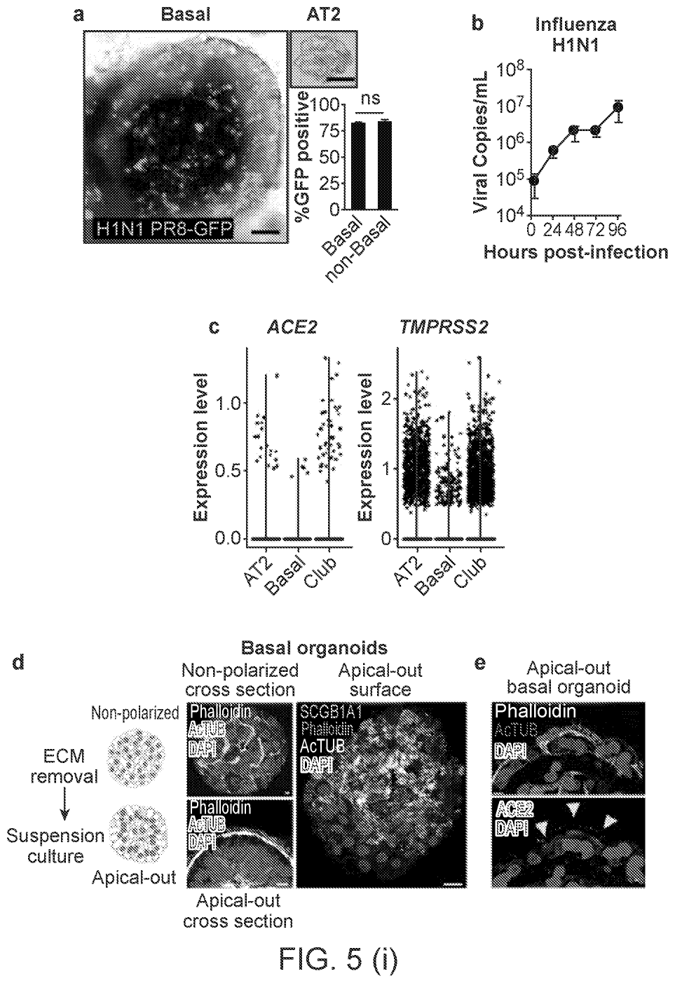

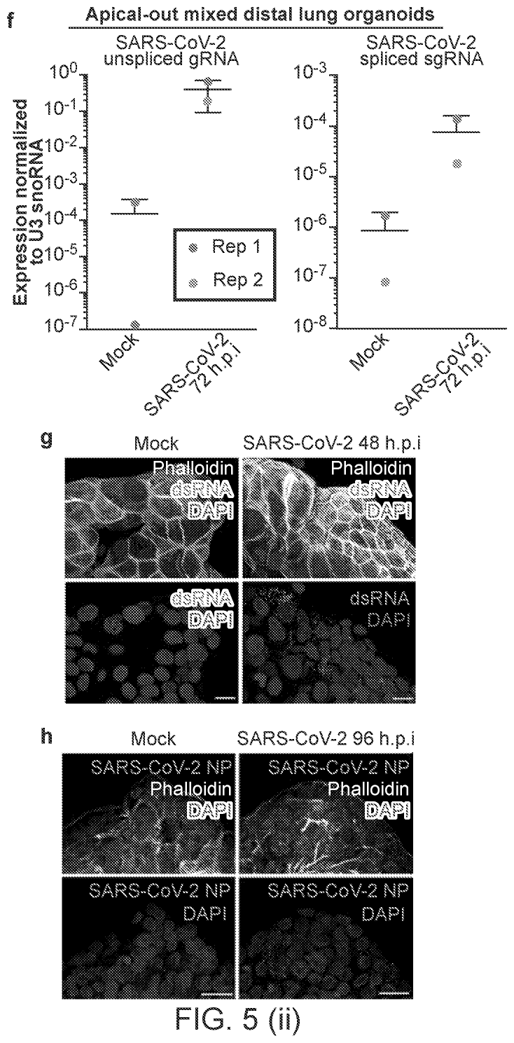

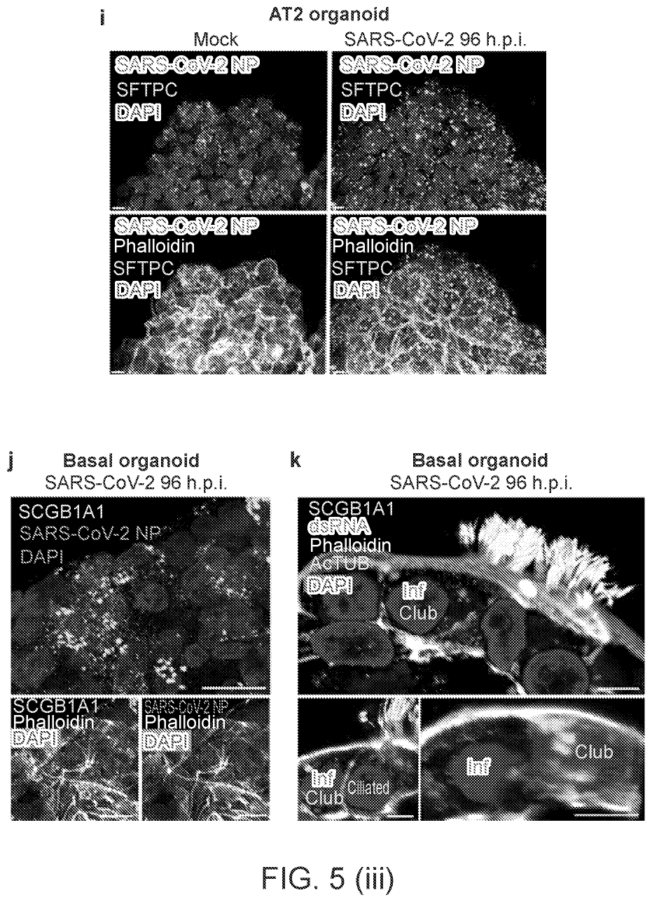

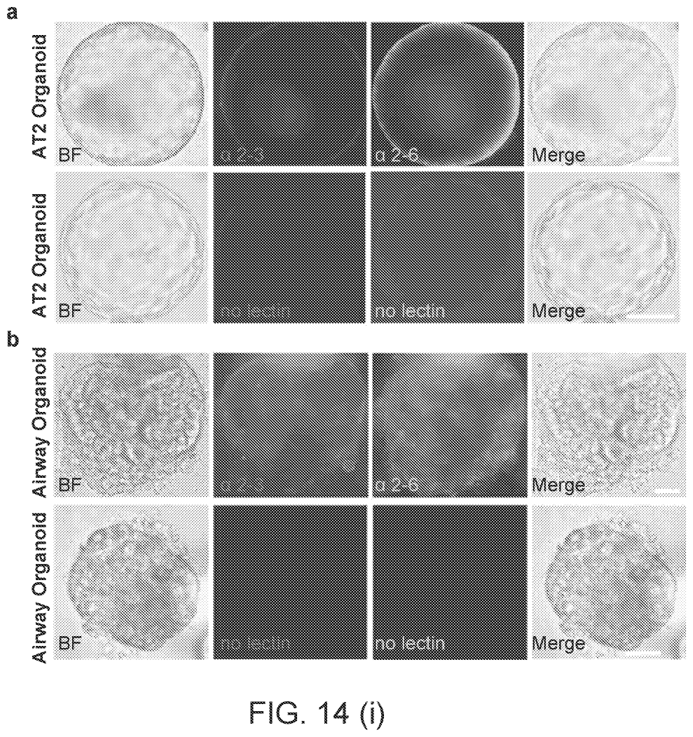

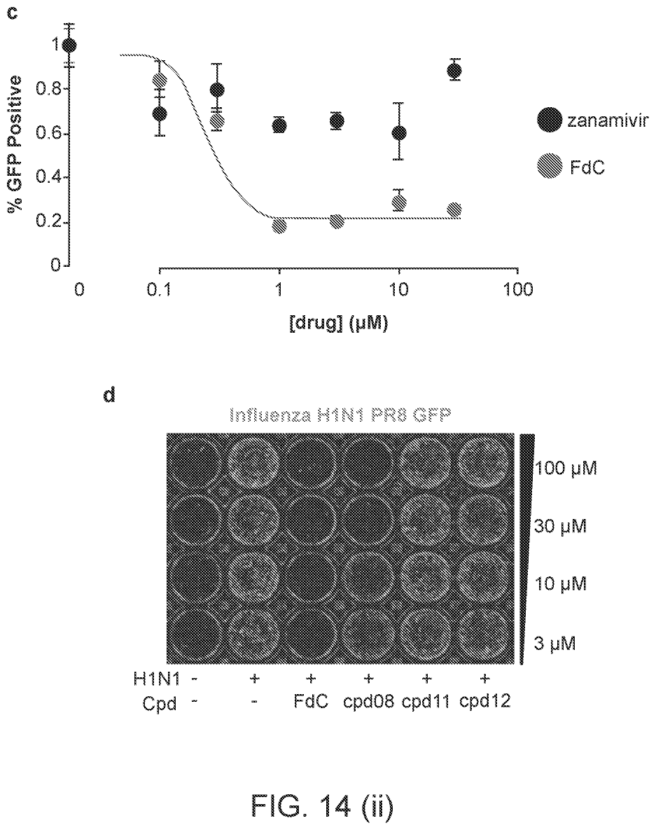

SARS-CoV-2 and influenza H1N1 infection of distal lung organoids. We next established the utility of distal lung organoids for human infectious disease modeling. Influenza virus strain H1N1 broadly injures both airway and alveolar epithelium. Both basal and AT2 organoids from human distal lung cultures were avidly infected by a recombinant influenza H1N1 PR8 strain expressing GFP upon viral replication (FIG. 5a) and viral genomic RNA accumulated in mixed organoid culture supernatants over a 96 hr time course (FIG. 5b), similar to previous reports with human lung airway organoids. Organoids also stained with lectins binding a2-3 and a2-6 sialic acid residues (FIG. 14a,b), indicating functional influenza receptors as in intact human lung. Pretreatment of lung organoids with zanamivir, which selectively targets influenza release from infected cells, did not inhibit H1N1 viral infection or replication, as shown by the lack of GFP attenuation. In contrast, the nucleoside analog 2′fluoro 2′deoxycytidine (FdC), which impairs replication across many virus families, was efficacious with EC₅₀ consistent with previous reports (FIG. 14c). Screening of diverse antiviral compound classes in H1N1-infected organoids in a 48-well format assay, quantified by GFP, revealed differential effectiveness (FIG. 14d), suggesting utility for scalable therapeutics discovery.

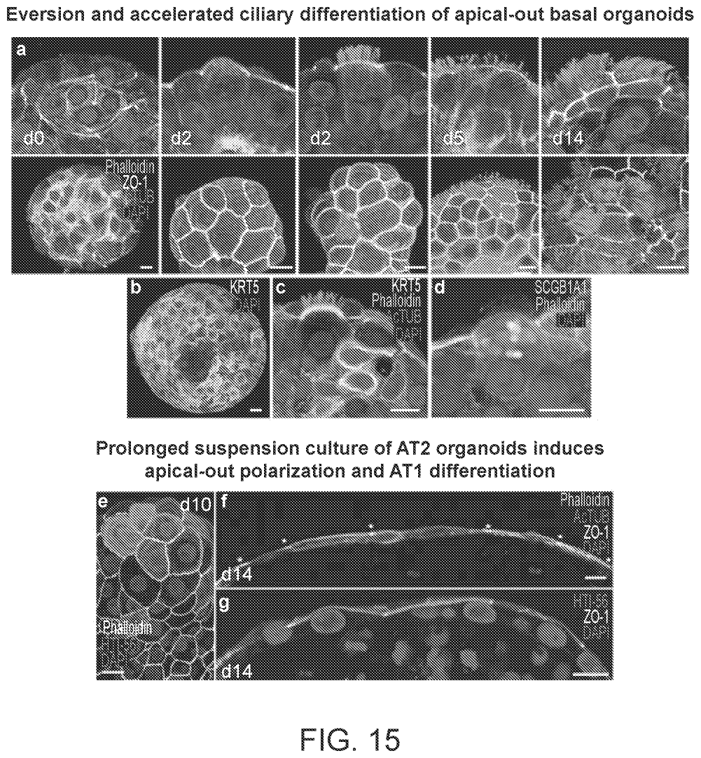

The COVID-19 pandemic is particularly significant for distal lung disease, where involvement of alveoli and terminal bronchioles elicits life-threatening pneumonia and respiratory failure. SARS-CoV-2 infects intestinal organoids, and 2D ALI monolayer cultures from upper airway, trachea and alveoli but efficient infection of distal lung tissue has not been demonstrated. In mixed distal lung organoids, scRNA-seq (FIG. 1q,r) revealed mRNA encoding the SARS-CoV-2 receptor ACE2 and the processing protease TMPRSS2 predominantly in club and AT2 cells (FIG. 5c), consistent with ACE2 expression in KRT5⁻ differentiated lumenal cells (FIG. 2j). The present long-term 3D basal and alveolar organoids are typically oriented with the basolateral surface oriented outwards, i.e. facing the extracellular matrix substratum, which could hinder SARS-CoV-2 infection of the apical ACE2-expressing luminal surface. We previously described rapid inversion of apical-basolateral polarity of gastrointestinal organoids by removal from the extracellular matrix gel and growth in suspension, robustly generating organoids with their apical surfaces oriented outward (apical-out), and thus greatly facilitating host-pathogen interactions on the lumenal surface. We adapted this method to distal lung organoids where suspension culture rapidly induced apical-out polarization and differentiation. Within 48 h non-polarized KRT5⁺ organoids reorganized into apical-out epithelial spheroids with microvilli, apical junctions, and some motile cilia facing the organoid exterior. Within 5 days, differentiation of outwardly oriented ciliated cells continued to accelerate and was progressive over weeks (FIG. 5d; FIG. 15a-c). Additionally, everted organoids displayed outwardly facing club cells with apical secretory granules (FIG. 5d; FIG. 15d); delayed appearance of AT1 cells was also observed in alveolar organoids (FIG. 15e-g). In apical-out organoids in suspension culture, ACE2 was detected on the apical membrane of cells on the external organoid surface (FIG. 5e) versus restricted to the internal differentiated apical lumen in basal organoids maintained in ECM (FIG. 2j).

Notably, SARS-CoV-2 readily infected apical-out mixed distal lung organoids. SARS-CoV-2 genomic RNA was detected at 72 h post-infection by qPCR at levels similar to the abundantly expressed ubiquitous U3 snoRNA (FIG. 5f, left). Infection was further confirmed by the presence of replication-specific SARS-CoV-2 subgenomic RNA (sgRNA) (FIG. 5f, right) and by production of infectious virions capable of plaque formation on VeroE6 cells: 35 PFU/ml from cellular lysates and 65 PFU/ml from cellular supernatants. Immunofluorescence visualization of SARS-CoV-2-infected basal organoids revealed the sequential appearance of double-stranded RNA (dsRNA) by 48h, reflecting viral genome replication (FIG. 5g), and of SARS-CoV-2 nucleocapsid protein (NP) by 96h (FIG. 5h). We then examined which organoid cell types were infected by SARS-CoV- 2 in vitro. Approximately 10% of AT2 organoids exhibited evidence of infection with prominent localization of SARS-CoV-2 NP within SFTPC-expressing cells (FIG. 5i). Similarly, SARS-CoV-2 infected -10% of basal organoids. Since basal organoids contain multiple cell types, immunostaining of dsRNA or SARS-CoV2-2 NP was overlaid with either KRT5, SCGB1A1, or AcTUB to determine infected cell identity. In 2,621 total cells representing cultures from 4 individuals, SARS-CoV-2 infection was not detected in KRT5⁺ basal or AcTUB⁺ ciliated cells (odds ratio 0, p-value <0.05), in contrast to prior studies in 2D ALI culture where SARS-CoV-2 infected upper airway ciliated cells. However, SARS-CoV-2 NP and dsRNA immunofluorescence signals were primarily present in SCGB1A1⁺ club cells (FIG. 5j-k) which were strongly associated with and accounted for 79% of NP/dsRNA-positive cells (odds ratio 19.33, p<0.0001); 21% of infected cells lacked SCGB1A1 (FIG. 5j-k). Overall, these studies indicated direct SARS-CoV-2 infection of AT2 cells, and implicated club cells as a novel target population.

The slow cellular turnover of adult lung epithelium has hampered identification of regional stem cell populations. Lack of long-term culture systems and substantial differences between mouse and human lung have particularly impaired validation of human distal lung stem cells. Here, we described a robust, feeder-free, chemically defined method for long-term human distal lung airway and alveolar clonogenic organoid growth, which was applied to progenitor discovery and infectious disease modeling.

Basal cells perform crucial stem cell functions in lung and other tissues. Although lung molecular and histochemical basal cell subsets have been reported and differential progenitor activity has been observed in mouse, proximal human airway studies suggest uniform basal cell proliferative activity in vivo. We identified two interrelated molecular subtypes of KRT5⁺ human distal lung basal cells, Basal 1 and Basal 2; although we cannot exclude squamatization in the latter, proliferation selectively localized to the former. Notably, a TNFRSF12A^{hi} fraction within the Basal 1 subset, by both mRNA and protein criteria, possessed enriched clonogenic progenitor activity, establishing functional precedent for a proliferation-enriched basal cell subtype. TNFRSF12A⁺ basal cells often but not exclusively localized to bases and tips of airway furrows, possibly representing a distinct airway progenitor niche, as proposed for goblet cells. Conceivably, TNFRSF12A or other markers could distinguish analogous human basal cell progenitor subsets in other tissues.

The differentiation of iPSC to lung epithelial lineages, while efficacious, can be constrained by efficiency, feeder dependence, and fetal gene expression, necessitating methods to expand resident adult lung stem cells. Prior human lung basal cell cultures (nasal cavity, trachea, proximal bronchi) achieved in vitro proliferation and clonal expansion, ciliated and mucous differentiation and reconstitution of denuded rat tracheal epithelium but have been limited by short term culture, feeder dependence, and restriction to upper airway. Further, intrinsic differences in basal cells and their differentiated progeny along the lung proximal-distal axis may limit generalizability of upper airway studies to distal airways and alveoli. Our distal airway basal organoids represent amongst the most significant clonal expansion of basal cells from any region of the human lung.

Culture of human adult AT2 cells has been characteristically short-lived and feeder-dependent. The present clonogenic, long-term, feeder-free and chemically defined human alveolar organoid cultures uniformly expressed the canonical AT2 genes SFPTA1/B/C, possessed characteristic lamellar bodies, functionally required autocrine WNT signaling, and transdifferentiated to AT1 cells upon culture on glass or in suspension. Variable AT2 subpopulations were marked by LYZ or MUC5B (FIG. 10-6), previously identified in mouse and implicated in Idiopathic Pulmonary Fibrosis, respectively. Murine lung progenitors with airway and alveolar epithelial differentiation capacity include LNEPs, Distal Airway Stem Cells (DASCs) and Bronchioalveolar Stem Cells (BASCs). We did not observe clonal organoids simultaneously containing both mature airway and alveolar cell types but cannot exclude such bipotential progenitors upon different culture conditions.

We also describe a facile model for SARS-CoV-2 infection of distal lung, as relevant for COVID-19-associated pneumonia and ARDS. Intestinal organoids recapitulate SARS-CoV-2 infection, 2D ALI monolayer cultures from upper airway/trachea organoids are infected by SARS-CoV-2 and a comprehensive study of 2D ALI cultures over the entire proximal-distal airway axis revealed a descending susceptibility gradient. However, these studies neither employed 3D lung organoid systems nor demonstrated efficient infection of distal lung tissue. Previously, intestinal organoids were mechanically sheared to allow apical SARS-CoV-2 infection. In contrast, we describe robust SARS-CoV-2 infection by everting distal lung organoids in suspension, rapidly creating physiologically relevant “apical-out” cultures that facilitate direct infection of the ACE2-expressing exterior apical surface that recapitulates in vivo physiology. Active SARS-CoV-2 infection of basal and AT2 organoids was evidenced by detection of spliced subgenomic viral RNA, dsRNA replication intermediates, viral nucleocapsid protein and infectious virus production. In addition to AT2 organoid infection, our results implicate SCGB1A1⁺ club cells as a novel SARS-CoV-2 distal lung target whose infection could compromise protective lung protective glycosaminoglycans, thus facilitating a vicious COVID-19 infection cycle. Although we did not observe ciliated cell infection, SCGB1A1-negative populations were also infected and are under further investigation; for example, bronchial transient secretory cells express ACE2 and TMPRSS2. Since SARS-CoV-2 infects cultured upper airway ciliated cells in vitro, cognate distal lung ciliated cells could be less susceptible to SARS-CoV-2 or their infection facilitated by alternative culture conditions. These studies extend our prior pathogen investigations in apical-out GI organoids, and the strong induction of functional ciliogenesis in apical-out suspension conditions may allow improved expansion of ciliated cells versus current 2D ALI monolayer protocols.

Overall, single cell analysis of organoid cultures, as exemplified here, represent a general strategy for human stem cell investigation in slowly proliferating tissues. The culture of progenitors for all adult distal lung epithelial lineages, including alveoli, enables human pulmonary disease modeling including neoplastic and interstitial lung diseases and allows tissue engineering and precision medicine applications. Finally, this organoid system facilitates general mechanistic and therapeutic investigations of pulmonary pathogens, including the SARS-CoV-2 distal lung infection associated with fulminant respiratory failure.

### REFERENCES

Zhu, N., et al. A Novel Coronavirus from Patients with Pneumonia in China, 2019. N Engl J Med 382, 727-733 (2020).

Wang, D., et al. Clinical Characteristics of 138 Hospitalized Patients With 2019 Novel Coronavirus-Infected Pneumonia in Wuhan, China. Jama 323, 1061-1069 (2020).

Graham, R. L. & Baric, R. S. SARS-CoV-2: Combating Coronavirus Emergence. Immunity 52, 734-736 (2020).

Rawlins, E. L., et al. The role of Scgb1a1+ Clara cells in the long-term maintenance and repair of lung airway, but not alveolar, epithelium. Cell Stem Cell 4, 525-534 (2009).

Hong, K. U., Reynolds, S. D., Giangreco, A., Hurley, C. M. & Stripp, B. R. Clara cell secretory protein-expressing cells of the airway neuroepithelial body microenvironment include a label-retaining subset and are critical for epithelial renewal after progenitor cell depletion. American journal of respiratory cell and molecular biology 24, 671-681 (2001).

Barkauskas, C. E., et aL Type 2 alveolar cells are stem cells in adult lung. J Clin Invest 123, 3025-3036 (2013).

Desai, T. J., Brownfield, D. G. & Krasnow, M. A. Alveolar progenitor and stem cells in lung development, renewal and cancer. Nature 507, 190-194 (2014).

Chapman, H. A., et al. Integrin alpha6beta4 identifies an adult distal lung epithelial population with regenerative potential in mice. J Clin Invest 121, 2855-2862 (2011).

Vaughan, A. E., et al. Lineage-negative progenitors mobilize to regenerate lung epithelium after major injury. Nature 517, 621-625 (2015).

Zuo, W., et al. p63(+)Krt5(+) distal airway stem cells are essential for lung regeneration. Nature 517, 616-620 (2015).

Ray, S., et al. Rare SOX2+ Airway Progenitor Cells Generate KRTS+ Cells that Repopulate Damaged Alveolar Parenchyma following Influenza Virus Infection. Stem cell reports 7, 817-825 (2016).

Kim, C .F., et al. Identification of bronchioalveolar stem cells in normal lung and lung cancer. Cell 121, 823-835 (2005).

Liu, Q., et al. Lung regeneration by multipotent stem cells residing at the bronchioalveolar-duct junction. Nat Genet 51, 728-738 (2019).

Hogan, B. L., et al. Repair and regeneration of the respiratory system: complexity, plasticity, and mechanisms of lung stem cell function. Cell Stem Cell 15, 123-138 (2014).

Hong, K. U., Reynolds, S. D., Watkins, S., Fuchs, E. & Stripp, B. R. Basal cells are a multipotent progenitor capable of renewing the bronchial epithelium. Am J Pathol 164, 577-588 (2004).

Rock, J. R., et al. Basal cells as stem cells of the mouse trachea and human airway epithelium. Proc Natl Acad Sci U S A 106, 1 2771-1 2775 (2009).

Mou, H., et al. Dual SMAD Signaling Inhibition Enables Long-Term Expansion of Diverse Epithelial Basal Cells. Cell Stem Cell 19, 217-231 (2016).

Kretzschmar, K. & Clevers, H. Organoids: Modeling Development and the Stem Cell Niche in a Dish. Dev Cell 38, 590-600 (2016).

Hegab, A. E., et aL Isolation and in vitro characterization of basal and submucosal gland duct stem/progenitor cells from human proximal airways. Stem Cells Transl Med 1, 719-724 (2012).

Kumar, P. A., et al. Distal airway stem cells yield alveoli in vitro and during lung regeneration following H1N1 influenza infection. Cell 147, 525-538 (2011).

Zhou, J., et al. Differentiated human airway organoids to assess infectivity of emerging influenza virus. Proc Natl Acad Sci U S A 115, 6822-6827 (2018).

Tata, P. R. & Rajagopal, J. Plasticity in the lung: making and breaking cell identity. Development 144, 755-766 (2017).

Sachs, N., et al. Long-term expanding human airway organoids for disease modeling. EMBO J (2019).

Barkauskas, C. E., et al. Lung organoids: current uses and future promise. Development 144, 986-997 (2017).

Zacharias, W. J., et aL Regeneration of the lung alveolus by an evolutionarily conserved epithelial progenitor. Nature 555, 251-255 (2018).

Dye, B. R., et al. In vitro generation of human pluripotent stem cell derived lung organoids. Elife 4(2015).

Chen, Y. W., et al. A three-dimensional model of human lung development and disease from pluripotent stem cells. Nat Cell Biol 19, 542-549 (2017).

Yamamoto, Y., et al. Long-term expansion of alveolar stem cells derived from human iPS cells in organoids. Nat Methods (2017).

Jacob, A., et al. Differentiation of Human Pluripotent Stem Cells into Functional Lung Alveolar Epithelial Cells. Cell Stem Cell 21, 472-488 e410 (2017).

Anchang, B., et al. Visualization and cellular hierarchy inference of single-cell data using SPADE. Nat Protoc 11, 1264-1279 (2016).

Yan, K. S., et al. Intestinal Enteroendocrine Lineage Cells Possess Homeostatic and Injury-Inducible Stem Cell Activity. Cell Stem Cell 21, 78-90 e76 (2017).

Van der Velden, J. L., Bertoncello, I. & McQualter, J. L. LysoTracker is a marker of differentiated alveolar type II cells. Respir Res 14, 123 (2013).

Dobbs, L. G. Isolation and culture of alveolar type II cells. Am J Physiol 258, L134-147 (1990).

Nabhan, A. N., Brownfield, D. G., Harbury, P. B., Krasnow, M. A. & Desai, T. J. Single-cell Wnt signaling niches maintain stemness of alveolar type 2 cells. Science 359, 1118-1123 (2018).

Kajiji, S., Tamura, R. N. & Quaranta, V. A novel integrin (alpha E beta 4) from human epithelial cells suggests a fourth family of integrin adhesion receptors. EMBO J 8, 673-680 (1989).

van der Flier, L. G., Haegebarth, A., Stange, D. E., van de Wetering, M. & Clevers, H. OLFM4 Is a Robust Marker for Stem Cells in Human Intestine and Marks a Subset of Colorectal Cancer Cells. Gastroenterology (2009).

Stange, D. E., et al. Differentiated troy(+) chief cells act as reserve stem cells to generate all lineages of the stomach epithelium. Cell 155, 357-368 (2013).

Whitfield, M. L., George, L. K., Grant, G. D. & Perou, C. M. Common markers of proliferation. Nat Rev Cancer 6, 99-106 (2006).

Luca, V. C., et al. Notch-Jagged complex structure implicates a catch bond in tuning ligand sensitivity. i Science 355, 1320-1324 (2017).

Pardo-Saganta, A., et al. Injury induces direct lineage segregation of functionally distinct airway basal stem/progenitor cell subpopulations. Cell Stem Cell 16, 184-197 (2015).

Rock, J. R., et aL Notch-dependent differentiation of adult airway basal stem cells. Cell Stem Cell 8, 639-648 (2011).

Mori, M., et al. Notch3-Jagged signaling controls the pool of undifferentiated airway progenitors. Development 142, 258-267 (2015).

Karaca, G., et al. TWEAK/Fn14 signaling is required for liver regeneration after partial hepatectomy in mice. PLoS One 9, e83987 (2014).

Quinton, P. M. Both Ways at Once: Keeping Small Airways Clean. Physiology (Bethesda) 32, 380-390 (2017).

Stinson, S. F., etal. Epithelial and surfactant changes in influenzal pulmonary lesions. Arch Pathol Lab Med 100, 147-153 (1976).

Manicassamy, B., et al. Analysis of in vivo dynamics of influenza virus infection in mice using a GFP reporter virus. Proc Natl Acad Sci U S A 107, 11531-11536 (2010).

Imai, M. & Kawaoka, Y. The role of receptor binding specificity in interspecies transmission of influenza viruses. Curr Opin Virol 2, 160-167 (2012).

Kumaki, Y., Day, C. W., Smee, D. F., Morrey, J. D. & Barnard, D. L. In vitro and in vivo efficacy of fluorodeoxycytidine analogs against highly pathogenic avian influenza H5N1, seasonal, and pandemic H1N1 virus infections. Antiviral research 92, 329-340 (2011).

Zang, R., etal. TMPRSS2 and TMPRSS4 promote SARS-CoV-2 infection of human small intestinal enterocytes. Sci Immunol 5(2020).

Lamers, M. M., et al. SARS-CoV-2 productively infects human gut enterocytes. Science (2020).

Zhou, J., et al. Infection of bat and human intestinal organoids by SARS-CoV-2. Nat Med (2020).

Hou, Y. J., et al. SARS-CoV-2 Reverse Genetics Reveals a Variable Infection Gradient in the Respiratory Tract. Cell (2020).

Co, J. Y., et al. Controlling Epithelial Polarity: A Human Enteroid Model for Host-Pathogen Interactions. Cell Rep 26, 2509-2520 e2504 (2019).

Watson, J. K., et al. Clonal Dynamics Reveal Two Distinct Populations of Basal Cells in Slow-Turnover Airway Epithelium. Cell Rep 12, 90-101 (2015).

Nakajima, M., etal. Immunohistochemical and ultrastructural studies of basal cells, Clara cells and bronchiolar cuboidal cells in normal human airways. Pathology international 48, 944-953 (1998).

Baldwin, F. Basal cells in human bronchial epithelium. The Anatomical record 238, 360-367 (1994).

Vieira Braga, F. A., et aL A cellular census of human lungs identifies novel cell states in health and in asthma. Nat Med 25, 1153-1163 (2019).

Hajj, R., et al. Basal cells of the human adult airway surface epithelium retain transit-amplifying cell properties. Stem Cells 25, 139-148 (2007).

Teixeira, V. H., et al. Stochastic homeostasis in human airway epithelium is achieved by neutral competition of basal cell progenitors. Elife 2, e00966 (2013).

Mou, H. M., et al. Dual SMAD Signaling Inhibition Enables Long-Term Expansion of Diverse Epithelial Basal Cells. Cell Stem Cell 19, 217-231 (2016).

Treutlein, B., et al. Reconstructing lineage hierarchies of the distal lung epithelium using single-cell RNA-seq. Nature 509, 371-375 (2014).

Seibold, M. A., et al. A common MUCSB promoter polymorphism and pulmonary fibrosis. N Engl J Med 364, 1503-1512 (2011).

Hunninghake, G.M. A new hope for idiopathic pulmonary fibrosis. N Engl J Med370, 2142-2143 (2014).

Kathiriya, J. J., Brumwell, A. N., Jackson, J. R., Tang, X. & Chapman, H. A. Distinct Airway Epithelial Stem Cells Hide among Club Cells but Mobilize to Promote Alveolar Regeneration. Cell Stem Cell 26, 346-358 e344 (2020).

Lee, J. H., et al. Anatomically and Functionally Distinct Lung Mesenchymal Populations Marked by Lgr5 and Lgr6. Cell 170, 1149-1163 e1112 (2017).

Lukassen, S., et al. SARS-CoV-2 receptor ACE2 and TMPRSS2 are primarily expressed in bronchial transient secretory cells. EMBO J 39, e105114 (2020).

Clevers, H. Modeling Development and Disease with Organoids. Cell 165, 1586-1597 (2016).

Nikolic, M. Z. & Rawlins, E. L. Lung Organoids and Their Use To Study Cell-Cell Interaction. Current pathobiology reports 5, 223-231 (2017).

Yan, K. S., et al. Non-equivalence of Wnt and R-spondin ligands during Lgr5+intestinal stem-cell self-renewal. Nature 545, 238-242 (2017).

Chang, J., et al. Gpr124 is essential for blood-brain barrier integrity in central nervous system disease. Nat Med 23, 450-460 (2017).

Nagendran, M. R., D. P.; Harbury, P. B.; Desai, T. J. Automated cell type classification in intact tissues by single-cell molecular profiling In Revision (eLife). See Supplementary Note 1 for Manuscript.

Van Lidth de Jeude, J. F., Vermeulen, J. L., Montenegro-Miranda, P. S., Van den Brink, G. R. & Heijmans, J. A protocol for lentiviral transduction and downstream analysis of intestinal organoids. J Vis Exp (2015).

Krafft, A. E., et al. Evaluation of PCR testing of ethanol-fixed nasal swab specimens as an augmented surveillance strategy for influenza virus and adenovirus identification. J Clin Microbiol 43, 1768-1775 (2005).

Carpenter, A. E., et al. CellProfiler: image analysis software for identifying and quantifying cell phenotypes. Genome Biol 7, R100 (2006).

### Methods

### Human Tissue Procurement and Processing

SARS-CoV-2 and influenza H1N1 infection of distal lung organoids. All material used in this work was approved by the Stanford School of Medicine's Institutional Review Board and performed under protocol #28908. Standard informed consent for research was obtained in writing prior to tissue procurement. Peripheral lung tissue within 1 cm of the visceral pleura was obtained from surgical discards from lobectomies. For patients with suspected lung cancer, cases with clinical T4 (American Joint Cancer Committee 6th edition) disease (e.g. features such as bronchial invasion or parenchymal satellite nodule/ metastases) were not used. Normal tissue was harvested from the lung margin most anatomically distal to palpably well-defined lesions, or from uninvolved lobes in the case of pneumonectomies. Samples with tumors containing ill-defined margins were deferred. Tissue was either processed fresh or placed at 4° C. overnight and processed the following morning.

Organoid culture. To isolate distal airway cells, lung parenchyma 1 cm from the visceral pleura was mechanically dissociated with Castro scissors, washed and incubated with 5 Units/ml porcine elastase (Worthington), 100 Kunitz Units/ml DNase I (Worthington), and Normocin (InvivoGen) resuspended in two tissue volumes of lung organoid media, comprised of Advanced DMEM/F12 (Invitrogen) supplemented with 10 mM nicotinamide, n-acetyl cysteine, 1X B27 supplement minus vitamin A, recombinant human NOGGIN (100 ng/ml, R&D Systems), recombinant human EGF (50 ng/ml, R&D Systems), and TGF-beta inhibitor A83-01 (100nM, Tocris). The tissue was then agitated for one hour at 37C and the resultant cell suspension was filtered through 100 through 40 μm cell strainers and subjected to ammonium chloride red blood cell lysis. The cell pellet was then washed and resuspended in 10 volumes of reduced growth factor Basement Membrane Extract II (Trevigen). Cells in matrix were then plated in 24-well plates in 50 microliter droplets, and warm media was added after the droplets solidified for ten minutes at room temperature Media was changed every 3-4 days and organoids were passaged every 3-4 weeks by dissociation with TrypLE. Passaging was based on ECM durability/integrity and estimated organoid confluency, judged by estimated organoid volume to volume of the ECM droplet. To rule out contamination by malignant cells, long-term cultures were systematically evaluated for the presence of dysplasia or carcinoma by a board-certified pathologist. In addition, five long-term organoid cultures (2-6 months) underwent targeted Next Generation Sequencing to detect the presence of pathogenic variants (see below). Full details are provided in Supplementary Methods.

Tandem MACS stromal depletion and EPCAM purification of distal lung cells. Distal lung was dissociated as above, and all incubation steps were carried out on ice. 10⁷ cells were incubated with Fc Block (Biolegend 422301) and diluted 1:100 in FACS buffer (2 mM EDTA and 0.2% fetal calf serum in 1X PBS pH 7.4), for ten minutes followed by APC conjugated anti-CD45 antibodies at 1 pg/ml in FACS buffer for 30 minutes, washed, and subjected to two rounds of depletion with magnetic beads according to manufacturer's protocol (Miltenyi: anti-human fibroblast 130-050-601, anti-CD31 130-091-935, anti-APC 130-090-855, LS column 130-042-401). Unlabeled cells were then centrifuged at 300×g and labeled with a cocktail of 1 pg/ml of PerCP-Cy5.5 anti-EPCAM antibody and Zombie Aqua viability stain (Biolegend 423101) diluted 1:400 from stock concentration in FACS buffer.

Organoid Cryopreservation and Recovery. For cryopreservation and recovery, ECM droplets were dissociated by pipetting in 3 volumes of PBS with 5 mM EDTA and then incubated on ice for one hour. Cells were pelleted at 300×g for 5 minutes and resuspended in freezing medium (fetal bovine serum (Gibco), 10% v/v DMSO), placed into cryovials and then into Mr. Frosty™ (Thermo Fisher) containers and stored in a −80C freezer overnight, followed by transfer to liquid nitrogen vapor phase for long term storage. Organoids were recovered by quick thaw in a 37C water bath followed by washing in organoid media and plating in ECM with organoid media plus 10 μM ROCK inhibitor Y-27632 (Tocris).

Screening exogenous growth factors in organoid culture. Distal airway cells were isolated and plated as above with the following exceptions: ADMEM/F12 was used instead of organoid medium during elastase digestion of lung tissue, cells were serially diluted and filtered through a 40 micron cell strainer and counted with a hemocytometer. 1000 viable epithelial cells (by Trypan blue exclusion, size, and morphology) per μL ECM were plated per 5 μL Matrigel droplet per well. Base media consisted of organoid media lacking A83-01, EGF, NOGGIN, WNT3A or RSPO1. EGF (final 50 ng/ml, R&D), NOGGIN (final 100 ng/ml, R&D), WNT3A (final 100 ng/ml, R&D), RSPO1 (final 500 ng/ml, Peprotech) or the PORCUPINE inhibitor C59 (final 1μM, Biogems) were added singly or in combination to base media. Images were obtained ten days after primary plating with an inverted light microscope at 5X magnification. Each condition was plated in quadruplicate and organoid formation was quantified using the analyze particle (threshold=4902 pixels) plugin in ImageJ as previously described.

Single Cell RNA-seq of unfractionated organoid cultures. Lung organoid cultures from separate individuals were dissociated 4 weeks after primary plating and subjected to droplet based scRNA-seq with the 10× Genomics Gemcode Single Cell 3′ platform with a 5 nucleotide UMI according to manufacturer's protocol. Cell capture, library preparation, and sequencing were performed as previously described.

Single cell RNA-seq of purified AT2 organoid cultures. LysoTracker+ AT2 cells from unfractionated organoids were purified by FACS and cultured for two months with one passage. These were dissociated and subjected to droplet-based scRNA-seq with the 10× Genomics Chromium Single Cell 3′ platform v2 according to the manufacturer's protocol. The library was sequenced using paired-end sequencing (26 bp Read 1 and 98 bp Read 2) with a single sample index (8 bp) on an Illumina NextSeq 500. Data preprocessing and Principle Component Analysis were carried out with CellRanger v1.2.

Electron microscopy. Organoid cultures were fixed in ECM with 2.5% glutaraldehyde in 0.1 M cacodylate buffer (pH 7.4), dehydrated, embedded in epoxy resin and visualized with a JEOL (model JEM1400) transmission-electron microscope with a LaB6 emitter at 120 kV.

Histology and immunocytochemistry. Organoids were fixed with 2% paraformaldehyde at 4 degrees Celsius overnight, paraffin embedded and sectioned (10-20 μm) as previously described. Sections were deparaffinized and stained with H&E for histological analysis. Antibodies used for immunocytochemistry staining are listed in Supplementary Methods following standard staining protocol as previously described and images were acquired on a Leica-SP8 confocal microscope.

RNA fluorescent in situ hybridization. RNA in situ hybridization was performed according to Nagendran et al.

Whole mount organoid confocal immunofluorescence microscopy. Intact, uninfected organoids were fixed in 2% paraformaldehyde in 100 mM phosphate buffer (pH 7.4) (4% paraformaldehyde for infected organoids) for one hour at room temperature, washed with PBS with 100 mM glycine, permeabilized 0.5% triton X-100 in PBS for one hour, then incubated in staining buffer (4% BSA, 0.05% Tween-20 in PBS pH7.4, 10% goat/donkey serum) for an additional hour, followed by incubation with primary antibody for 24 hours at room temperature in staining buffer. Whole mounts were then washed with PBS-T and incubated with fluorescent secondary antibodies, phalloidin and DAPI, for four hours at room temperature in staining buffer. Following additional washes, whole mounts were submerged in mounting media (VECTASHIELD, Vector Laboratories) and mounted on chambered coverslips for imaging in four channels using Zeiss LSM 700 or 900 confocal microscopes. 3D rendering of confocal image stacks was performed using Volocity Image Analysis software (Quorum Technologies Inc., Guelph, Ontario). For FIG. 5j, requiring 5 colors, cilia were distinguished by staining with two fluorescent secondary antibodies and merging the colocalized voxels into a pseudocolored channel using Volocity software. Lectin staining (FITC-Sambuca Nigrin, Vector Labs FL-1301; Biotin-Maackia Amurensis, Vector Labs FL-1301) was carried out according to manufacturer's protocol after fixation of organoids with 0.1% paraformaldehyde in PBS for 1 hour at room temperature followed by blocking with Avidin/Biotin (Vector Labs SP-2001). Biotin-Maackia Amurensis lectin was labeled with streptavidin-PE conjugate (Thermo Fisher SA10041) and after washing lectin staining was imaged in a Keyence BZ-X700.

Next generation sequencing of organoid cultures. Ten organoid cultures were sequenced using a commercial targeted resequencing assay with end-to-end coverage of 131 cancer genes and companion software (TOMA COMPASS Tumor Mutational Profiling System, Foster City, Calif.) to determine the presence of oncogenic mutations in long-term organoid cultures. Libraries were sequenced on an Illumina NextSeq 500.

Density sedimentation of basal cells. Organoid cultures within 2-3 weeks of primary plating were dissociated with 1 U/ml neutral protease (Worthington, Cat LS02100) and 100 KU of DNase I in organoid media. Basal organoids were then collected by gravity sedimentation and the supernatant was either aspirated or collected for downstream use. Basal organoids were then further fractionated on a custom Ficoll-Paque gradient (4 vol Ficoll-Paque to 1 vol PBS) and centrifuged at 300×g for 10 minutes at room temperature. The supernatant was aspirated and the organoid pellet was resuspended in 10 ml PBS in a 15 ml conical tube, collected by gravity sedimentation, and plated into ECM as described above.

FACS isolation and culture of AT2 cells. Organoids were dissociated with TrypLE followed by neutralization with 10% volume fetal calf serum, subjected to DNase at 100 kU/ml, washed with organoid media and then incubated with 100 cell pellet volumes of organoid media with 10 nM LysoTracker Red DND-99 (Thermo Fisher L7528) at 37C for 30 minutes. Cells were then washed and resuspended in FACS buffer as described above, incubated with Fc block, followed by incubation on ice with labeling cocktail consisting of 1 μg/ml of PerCP-Cy5.5 anti-EPCAM antibody and Zombie Aqua viability stain (Biolegend 423101) diluted 1:400 from stock concentration in FACS buffer. EPCAM^{hi} and LysoTracker^{hi} cells were sorted into organoid media with 10 μM Y-27632 (Tocris 1254) and cultured in ECM and media with Y-27632 for 24 hours, followed by regular media. All FACS antibodies were purchased from Biolegend.

Color mixing studies with lentivirally transduced GFP and mCherry. FACS EPCAM⁺ stromal depleted organoids at d14 were infected with lentivirus at an estimated MOI of 0.9 according to Van Lidth de Jeude et al. with third generation lentiviral vectors (PGK-GFP T2A Puro, SBI cat# CD550A-1; mCherry modified from pLentiCRISPRv1 (Addgene #49545) to incorporate an EF-1a-mCherry P2A Puro cassette, a gift from Paul Rack). 96 hours after infection, organoids were treated with puromycin at a concentration of 600 ng/ml for 48 hours to select for transduced cells. Two weeks after selection, GFP expressing organoids and mCherry expressing organoids were dissociated to single cells and mixed in a 1:1 ratio and scored as monochromatic or mixed after 28 days of each passage. The same approach was employed for purified AT2 and basal cultures after respective purification strategies from an initial FACS EPCAM⁺ stromal depleted organoid starter culture.

Flow cytometry analysis of resident basal cells from adult human lung. Adult human lung tissue was procured and dissociated as above but cells were labeled with Zombie Aqua live:dead stain as above, washed with FACS buffer, and then fixed in 2% PFA in PBS overnight at 4C. Cells were then stained using the whole mount procedure as described above with the omission of PBS glycine washing. Fixed and permeabilized cells were then incubated with 1:400 dilution of Alexa Fluor 647 conjugated mouse anti-human cytokeratin 5 antibody (Abcam) for 24 hours at 4C in permeabilization buffer. Cells were then washed with FACS buffer and labeled with PE conjugated mouse anti-human TNFRSF12A antibody (clone ITEM-4, Biolegend) for 30 minutes on ice, followed by washing and analysis on a BD Aria Fusion instrument.

FACS isolation of TNFRSF12A^{hi} and TNFRSF12A^{neg} basal cells. Single cell suspensions from either fresh human distal lung or primary organoid culture at approximately 4 weeks of culture were dissociated as above, treated with Fc Block, and incubated in FACS buffer with Zombie Aqua 1:400, 1 μg/ml PerCP-Cy5.5 anti-human EpCAM (CD326), 1 μg/ml APC anti-human ITGA6 (CD49f), 2 μg/ml FITC anti-human ITGB4 (CD104), and 1 μg/ml PE anti-human TNFRSF12A (CD266). 30 minutes after labeling the cells were washed twice with FACS buffer and sorted for EPCAM^{hi}, ITGA6/ITGB4^{hi}, TNFRSF12A^{hi} and TNFRSF12A^{neg}. >5000 cells were sorted into Eppendorf tubes with lung organoid medium and 10 μM ROCK inhibitor Y-27632. All FACS antibodies were purchased from Biolegend.

Culture of TNFRSF12A^{hi} and TNFRSF12A^{neg} basal cells. Cells were seeded in ECM and submerged in lung organoid media with 10 82 M ROCK inhibitor Y-27632. Seeding density for cells FACS isolated from organoid culture was 1000 cells per well at a density of 100 cells/μL of ECM. Seeding for cells FACS isolated from fresh human distal lung was 3000 cells per well at a density of 300 cells/μL of ECM. After 24 hours, the media was changed to remove ROCK inhibitor and additionally changed every 72 hours. Organoid formation was manually quantified 14 days post plating by two independent observers.

NOTCH manipulation in TNFRSF12A^{hi} basal cells. TNFRSF12A^{hi} basal cells were isolated and cultured as above. 24 hours after plating, media was changed to lung organoid media with either vehicle (0.1% DMSO), 1 μg/ml JAG1 peptide (Anaspec), 500 nM soluble recombinant NOTCH receptor inhibitor Delta Like Ligand 4 mutant (DLL4_{E12}), or 1 μM gamma secretase inhibitor DBZ (Tocris). After 14 days of culture, biochemical estimation of proliferation was carried out by resazurin reduction assay (Alamar Blue, Thermo Fisher) for 16 hours according to manufacturer's protocol and resazurin reduction was measured via fluorescence readout on a Biotek Synergy H1 plate reader according to the manufacturer's protocol. Reference blank consisted of Alamar Blue reagent incubated in parallel media without cells. Expression and purification of the NOTCH receptor inhibitor DLL4_{E12}was performed as previously described.

H1N1 organoid influenza assay. Unfractionated cultures containing AT2, basal, and club cell types at 2-3 weeks were infected in triplicate with PR8 strain of H1N1 modified to express GFP upon viral replication after 24 hours of pretreatment with antiviral compounds. ECM was dispersed by addition of 5 mM EDTA in PBS, followed by washing and inoculation with GFP-reporter virus at an estimated MOI of 1 in media containing either vehicle or antivirals. After 12 hours (one influenza infection cycle), intact organoid GFP expression was visualized by either fluorescence microscopy with a Keyence BZ-X700 automated microscope, or dissociated to single cell, fixed with 0.1% PFA in PBS followed by flow cytometry quantitation of GFP⁺ cells. Antiviral dose response curves were generated using four-parameter nonlinear regression curve fitting with GraphPad Prism 7 (GraphPad Software, San Diego, CA). H1N1 tropism was assessed in a manner similar to above with the exception of Ficoll sedimented basal cell fraction versus non-basal fractions were dissociated to single cells, counted, and infected with an estimated MOI of 1 in organoid media for one hour at 37° C., followed by washing and reseeding into ECM, cultured for 16 hours, followed by dissociation and flow cytometry analysis as above.

Quantifying H1N1 infection productivity. Productivity of pandemic H1N1 virus infection (A/California/07/2009) was determined by qPCR according to Zhou et al but with the following modifications. Organoids at 6 weeks of culture were removed from ECM with 1 U/ml neutral protease, washed with media, and reseeded 1:1 in 24 well plate wells 10% ECM and organoid media for 24 hours. Virus was added at an estimated MOI of 0.01 and incubated for 2 hours at 37° C. The supernatant was removed and wells were washed thrice with media and incubated with 1 ml of media per well. 250 μL aliquots of cell culture supernatant were harvested at 2, 24, 48, 72 and 96 hour time points with an equal volume of media replaced for each aliquot. Virus was quantified by qPCR according to Krafft et al.

Suspension culture to generate apical-out polarity in lung organoids. Lung organoids grown embedded in 50 μl ECM-droplets were transferred to suspension culture as described in Co et al with some modifications. Briefly, ECM-embedded organoids were dislodged gently by pipetting using sterile LoBind tips (Eppendorf 22493008) and placed in 15 ml LoBind conical tubes (Eppendorf 30122216) containing ice-cold 5 mM EDTA in PBS. The ratio of EDTA solution to ECM and the time of solubilization is important to optimally release intact organoids from the matrix. 5 ml of EDTA solution are used per ECM-droplet (3 ECM droplets/15 ml conical) rotating for 1 h at 4° C. on a rotating platform. Organoids were centrifuged at 200×g for 3 min at 4° C. and the supernatant was removed. The pellet was re-suspended in growth media in ultra-low attachment 6-well tissue culture plates (Corning Costar 3471). Suspended organoids were incubated at 37° C. with 5% CO2 for different times (range 0-30 days) to characterize apical-out polarity, ciliogenesis, and differentiation, and to prepare apical-out organoids for infection experiments with SARS-CoV-2.

SARS-CoV2 infection of human distal lung organoids. VeroE6 cells were obtained from ATCC and maintained in supplemented DMEM with 10% FBS. SARS-CoV-2 (USA-WA1/2020) was passaged in VeroE6 cells in DMEM with 2% FBS. Titers were determined by plaque assay on VeroE6 cells using Avicel (FMC Biopolymer) and crystal violet (Sigma), viral genome sequence was verified, and all infections were done with passage 3 virus. Organoids were counted and passaged into suspension media for 6-8 days and then resuspended in virus media or an equal volume of mock media, at a MOI of 1 relative to total organoid cells in the sample, and then incubated at 37° C. under 5% CO2 for 2 hours. Organoids were then plated in suspension in EN media (apical-out organoids). At the indicated timepoints, organoids were washed with EN media and PBS and either resuspended in TRIzol LS (Thermo Fisher), freshly-made 4% PFA in PBS, or 250 μL EN media. Cells resuspended in EN media were lysed by freezing at −80. Culture supernatants were preserved in TRIzol LS or added directly to plaque assay monolayers. All SARS-CoV-2 work was performed in a class II biosafety cabinet under BSL3 conditions at Stanford University.

qPCR analysis of SARS-CoV-2 RNA. RNA from SARS-CoV-2-infected organoids was extracted by adding 750 μl TRIzol (Thermo Fisher Scientific), incubating at 55 ° C. for 5 min and then adding 150 μl chloroform. After mixing each sample by vortexing for 7 s, the samples were incubated at 25 ° C. for 5 min and then centrifuged at 12,000 r.p.m. for 15 min at 4 ° C. The aqueous layer was carefully removed from each sample, mixed with two volumes of 100% ethanol and purified using an RNA Clean & Concentrator-25 kit (Zymo Research) as per the manufacturer's instructions. All RNA samples were DNase treated with the Turbo DNA-free kit (Thermo Fisher Scientific). The Brilliant II SYBR Green QRT-PCR 1-Step Master Mix (VWR) was used to convert RNA to cDNA and amplify specific RNA regions on the CFX96 Touch real-time PCR detection system (Bio-Rad). RT reaction was performed for 30 min at 50 ° C., 10 min at 95 ° C., followed by two-step qPCR with 95 ° C. for 10 seconds and 55 ° C. for 30 seconds, for a total of 40 cycles. Two primer sets were used, either to amplify non-spliced SARS-CoV-2 genomic RNA (gRNA) spanning nucleotide positions 14221-14306, or spliced SARS-CoV-2 sgRNA.

Quantitation of SCGB1A1 and SFTPC mRNA expression in TNFRSF12A^{hi} basal cells. FACS-isolated basal cells cultured in the above conditions were fixed in 2% paraformaldehyde in PBS for one hour at room temperature, embedded in HistoGel (Thermo Fisher HG-4000-012), dehydrated and paraffin embedded en bloc. One hundred serial sections were obtained at 10 micron thickness, and immunocytochemistry and in situ hybridization were performed at each 100 micron level. Confocal images were acquired in a blinded manner and organoids were defined as a cluster of 3 or greater DAPI nuclei. Channels were acquired using identical parameters for DAPI, Alexa 488 (SFTPC RNA in situ hybridization), Texas Red (SCGB1A1 RNA in situ hybridization), and Alexa 647 (KRTS immunostaining). Z-stacks were collected and images were processed in ImageJ and maximum intensity Z projections were used to quantitate SCGB1A1 and SFTPC RNA in situ in Cell Profiler using the RNA Proximity Ligase Assay counting pipeline.

TNFRSF12A immunostaining of intact distal lung. Optimal staining of human distal lung tissue was achieved from specimens fixed within 30 minutes of primary surgical resections in 4% paraformaldehyde in PBS. Specimens were incubated in fixative overnight at 4° C., transferred to 30% sucrose, and embedded into OCT. 10 μm thick frozen sections were cut, subjected to citrate based antigen retrieval (Vector labs) at 70° C. for 30 minutes, followed by blocking for one hour with 10% goat serum in IF wash buffer as described above. Mouse anti-TNFRSF12A (clone ITEM-4, Biolegend) was utilized for (FIG. 4f-i) and polyclonal rabbit anti-TNFRSF12A (ThermoFisher PA5-20275) was used for FIG. 4a-e, h).

Live-imaging and confocal microscopy of immobilized apical-out lung organoids. Live organoids were held between two coverslips in a viewing chamber (Lab-Tek II two-chambered coverglass) and filmed using a Nikon TE2000E microscope using differential interference contrast (DIC) microscopy with a 63× objective. Samples were kept at 37° C. with 5% CO2 during imaging. Digital videos were collected by a Hamamatsu high-resolution ORCA-285 digital camera and rendered using OpenLab 5.5.2 software (Improvision). After recording, samples were fixed and stained without removal from the chambers and transferred to the confocal microscope for immunofluorescence microscopy.

Computational and Statistical Analysis of the scRNA-seq Data. scRNA-seq analysis was initially carried out on the data set with the greatest number of cells (Lung 1, 7,285 cells), and the same analysis were repeated on Lung 2 (4,512 cells) and Lung 3 (3,364 cells).

Data Preprocessing. Sample multiplexing, barcode processing, RNA-seq alignment, and 3′ gene counting were performed with the Cell Ranger Software Suitei (version 1.1). We used Seurat, an R toolkit developed for single cell analysis, to perform basic quality control of the data. Cells with less than 200 UMIs were filtered out, and genes expressed in less than 3 cells were filtered out. To further improve the quality of the data, we used the workflow recommended by Seurat on droplet-based scRNA-seq data to regress out (i) the effects of the total number of UMIs and (ii) the percentage mitochondrial gene counts. In addition, as suggested by the workflow, we used the gene dispersion analysis3 implemented in Seurat to select highly variable genes, retaining genes with logarithmic (base 10) mean expression between 0.05 and 5 and with logarithmic dispersion greater than 0.2. The quality control resulted in 7285 single cells and 1938 highly variable genes for unbiased analysis.

Unbiased Clustering and Visualization. First, we applied principle component analysis (PCA) to reduce the dimension of the data. The implementation of PCA in Seurat scaled the data to zero mean and unit variance on the log transformed data. The principle components (PCs) were sorted by their ability to explain the variance in the gene expression matrix. In order to select the number of PCs, we used the jackstraw procedure4, which is a built-in boot-strap procedure implemented in Seurat that resamples 1% of the data, re-runs PCA, and scores each PC accordingly. As part of the jackstraw procedure, the p value for each PC is computed by a proportion test comparing the number of genes with a p-value (testing the significance of association between a gene with the PC) below a particular threshold (default given by 10-5), compared with the number of genes expected under a uniform distribution of the gene p-values. We selected the top 23 PCs based on the increase from 10-23 to 10-19. We did not notice significant difference downstream with more PCs as they did not seem to capture additional signals we could interpret.

We applied unsupervised clustering to partition cell populations into groups to identify meaningful structures in the data. In particular, we applied a graph based clustering technique on the top 23 PCs (also implemented in Seurat), tailored for high-dimensional single-cell data. The method embeds cells in a K-nearest neighbor (KNN) graph and partitions the graph into highly interconnected cell communities5. The clustering algorithm requires a resolution parameter that determines the number of distinct cell populations. Varying the parameter from 0.1 to 0.5 yields 5 to 11 clusters representing cell identities defined at different resolutions.

To visualize the data, we used the t-Stochastic Neighbor Embedding (t-SNE) algorithms, as often done with scRNA-seq data, given its ability to capture non-linear relationships. We applied t-SNE on the significant PCs to visualize the cells in 2-D. In the t-SNE 2-D mapping (also referred to as the t-SNE space), each point corresponds to a cell, and cells that share similar global gene expression appear closer together in the 2-D map and cells that are different appear further apart. Furthermore, t-SNE enables us to visualize the expression levels of each cell for given genes, as well as groupings by technical batches or by clustering results.

Major Cell-type Annotation. The clustering algorithm is tunable and can result in grouping at different resolutions, or simply different number of groups. Therefore, we considered the clusters provided at a low resolution and a high resolution to determine the appropriate number of major cell types. In order to identify the major cell types that are present among the clusters, we performed differential gene analysis to identify cluster specific gene markers. We enumerated genes that are most up regulated in a given population relative to all the other populations. In particular, we computed the average of the log₁₀, expression of each gene among the cells assigned to a given cluster, and compared this average value to the average log₁₀, expression of the remaining cells in the data after normalization and size correction implemented in sSeq₇. After analyzing the top genes for each high-resolution cluster, we merged clusters that shared highly expressed genes and annotated 5 major populations, including basal cells, ATII cells and club cells.

Testing Canonical Marker Genes. Based on our experimental setup and staining experiments, we expected at least three populations (ATII, club and basal) to be present in the data. We wanted to see if the canonical markers for these cell types are significantly differentially expressed across the populations. However, because the expression levels of these same genes were part of the data used to identify cell clusters, naïve tests comparing the expression levels of these markers across clusters are not valid.

To work around this issue, we studied how the clusters we identify are dependent on the canonical cell type markers. First, we repeated the unsupervised clustering analyses using the exact same procedure described above on a scRNA-seq data excluding measurements for each of the marker genes. This yields clustering assignments that are “independent” of the marker gene (given the correlation that exists among the expression of many genes, we are not referring here to stochastic independence). We compared the low-resolution clusters (parameter 0.1 for the graph based clustering) obtained with and without each of the marker genes. After verifying that the clustering results were highly consistent according to multiple clustering concordant metrics, we used Kruskal-Wallis Rank Sum Test to test if a known marker gene is differentially expressed across all populations based on the clusters that did not utilize information of the marker gene (with the null hypothesis that the mean expression of a given gene is the same across all groups.)

Exploratory Analysis of Basal Cells. We found the basal cell population to be highly heterogeneous, so we analyzed the genes that appeared to be differentially expressed among the subpopulations. First, we identified from both t-SNE and clustering two distinct clusters that corresponded to a subpopulation of quiescent basal cells and a subpopulation of non-quiescent basal cells. To identify signatures of novel cell populations, we also searched for enriched markers that are surface markers. We intersected the most up-regulated or down-regulated genes with genes annotated with surface marker proteins (GO: 0031224, intrinsic component of membrane, Table 2). Among the identified genes, we found the gene TNFRSF12A to be highly differentially expressed and one that would be amenable to FACS purification. In addition, we noticed HES1 was also ranked as one of the top genes differentiating the non-quiescent basal population from the quiescent basal population.

Because TNFRSF12A and HES1 were identified by ranking differentially expressed genes in only one sample, we conducted the exact same pipeline on two independent biological samples, where we also easily identified the non-quiescent and quiescent basal cells. We took the gene rankings for the three samples and applied robust rank aggregations for the rank of each gene among the list. For each gene, the algorithm looks at how the gene is ranked in each list and compares this with the rankings to the baseline case where all the gene lists are randomly shuffled. As a result, each gene can be assigned a p-value, which we report as the significance of its rank.

Associating TNFRSF12A with Proliferative Markers among Basal Cells. To understand the role of TNFRSF12A, we investigated associations between expression levels of TNFRSF12A and known proliferative markers both experimentally and computationally. Experimentally, the canonical basal cells were FACS sorted by the simultaneous presence of ITGA6, ITGB4 and EPCAM, followed by separating this triple-positive population into TNFRSF12A_{neg} and TNFRSF12Aₕᵢ fractions which were independently cultured to measure cell growth. To computationally mimic this approach and explore its outcome we selected all cells in scRNA-seq that expressed basal markers, and divided the cells into three groups defined by the top (TNFRSF12Aₕᵢ), bottom (TNFRSF12Aₗₒ) and middle two (TNFRSF12A_{med}) quartiles of TNFRSF12A mRNA expression. Finally, we tested if the presence of a proliferative gene signatures (MKI67, MYBL2, PLK1, BUB1, E2F1, FOXM1) was associated with the these three groups using a Chi-square test for independence.

Computational Reproducibility. We provide a step-by-step guide on the scRNA-seq analysis used for this manuscript in R markdown html format using the R knitr package.

SPADE Analysis. To explore and visualize the differentiation hierarchy between the cells in the data, we used a similar approach based on SPADE (Spanning Tree Progression of Density-normalized events) which uses a minimum spanning tree (MST) to represent the hierarchical relationship between cells. Cells within or in nearby branches on the tree are hierarchically more related compared to distant cells. The tree was optimized from a set of 42 genes comprised of a mixture of 15 highly expressed cluster-specific genes derived from the top 3 genes enriched in each of the 5 major t-SNE clusters described above in addition to 27 high variable genes derived using the robust statistics of variability known as median absolute deviation (MAD) defined as median of the absolute deviations from the data's median. For each gene, the MAD score is estimated from the non-zero count data using R. The 27 high variable genes were derived from combining top genes with high MAD scores from cells within and between clusters using a supervised approach.

Running SPADE. SPADE analysis was performed using the R implementation₁₁, that runs on Mac OS X and requires an FCS data file input format created from asinh-transformed gene expression counts with cofactor of 5. The SPADE analysis involves running the following 4 major steps: (1) density-dependent down-sampling of single-cell data, (2) agglomerative clustering, (3) joining clusters with a minimum spanning tree and (4) up-sampling to map all cells onto the final output tree. Each node and node size in the MST output represents a median marker expression and the number of cells within that node respectively. SPADE was run using the default settings on an initial number of 200 clusters but with no density dependent down-sampling. To investigate the sensitivity of the tree layout, we performed 3 different random seeded SPADE analyses on the data set and visually compared the SPADE trees.

SPADE Analysis of Club and Basal cells. To investigate in more detail the hierarchical relationship between Basal and Club cells alone, a SPADE tree derived from using the same set of 42 genes described above was applied. The result is summarized in FIG. 8 which suggests potential intermediate states (outlined in purple) taking place in addition to the main Basal to Club transition (continuum from blue to red states) identified by visualizing the median expression profiles (FIG. 8) for some major cluster specific and transition genes on the SPADE tree.

Next Generation Sequencing of Organoid Cultures. To verify the absence of cancerous cells in organoid culture, genomic DNA was extracted from five long-term organoid cultures (range 4-8 weeks) from different individuals and sequenced using a commercial targeted resequencing assay for 130 cancer genes and software (TOMA tumor profiling system, Foster City, Calif.).

Organoid RNA Extraction, cDNA Synthesis, and qRT-PCR. Mixed human distal lung organoids (p0, day 28) were FACS sorted into TNFRSF12A-negative, -medium and -high fractions. RNA was extracted using the Arcturus PicoPure kit (Applied Biosystems, KIT0204). Extracted RNA was reverse-transcribed to cDNA using the iScript cDNA synthesis kit (Bio-Rad,1708841). SsoAdvanced PreAmp Supermix (Bio-Rad, 1725160) was used for pre-amplification. The following Taqman Gene Expression Assay were used (Thermo Fisher): GAPDH as endogenous control (Hs02786624_g1), Krt5 (Hs00361185_m1), ITGA6 (Hs01041011_m1), ITGB4 (Hs00236216_m1), TNFRSF12A (Hs00171993_m1) along with TaqMan™ Universal Master Mix II, no UNG (Applied Biosystems, 4440040). All samples were run in triplicate on Applied Biosystems 7900HT Fast Real-Time PCR System with cycle conditions: 50°C. for 2 min, 95°C. for 10 min, followed by 40 cycles of 95°C. for 15 sec, and 60°C. for 1 min. Fold change of gene expression of TNFRSF12A-negative, -medium and -high fractions compared to the TNFRSF12A-negative sample was calculated using the ΔΔCt method and presented in FIG. 14i.

RNA FISH using Proximity Ligation- in situ Hybridization (PLISH). RNA FISH on paraffin embedded sections was performed according to the protocol by Nagendran et al. for SFTPC and SCGB1A1.

Lung Organoid Media Components

| Component | Vendor | Cat # | [Final] | |
|---|---|---|---|---|
| HEPES | Gibco | 15630-080 | 1 | mM |
| GlutaMAX-1 (100x) | Gibco | 35050-061 | | |
| NIC (Nicotinamide) | Sigma | N3636 | 10 | mM |
| NAC (N-acetylcysteine) | Sigma | A9165-5G | 1 | mM |
| B-27 supplement (50x) | Gibco | 125870-01 | 1X | |
| A83-01 | Tocris | 2939 | 500 | nM |
| Pen/Strep Glutamine | Gibco | 10378016 | | |
| Human EGF | R&D | 236-EG-01M | 50 | ng/mL |
| Human Noggin | R&D | 6057-NG/CF | 100 | ng/mL |

| | | |
|---|---|---|
| Advanced DMEM/F-12 | Thermo | 12634-028 |
| | Fisher | |

Additional Medium Additives

| Component | Vendor | Cat # | [Final] | |
|---|---|---|---|---|
| Wnt-3a | R&D | 5036-WN | 100 | ng/mL |
| R-spondin 1 | Peprotech | 120-38 | 500 | ng/mL |
| WNT C-59 | Biogems | 1243913 | 1 | μM |

Immuno- and Lectin- fluorescence Reagents

| Anti-body | Vendor | Catalog # | Dilution |
|---|---|---|---|
| Mouse anti- | Terrace Biotech | TB-27AHT2-280 | 1:100 |
| HTII-280 | | | |
| Mouse anti- | Terrace Biotech | TB-29AHT1-56 | 1:100 |
| HTI-56 | | | |
| Rabbit anti- | Santa Cruz | sc-13979 | 1:100 |
| SFTPC | Biotechnology | | |
| Mouse anti- | Sigma Aldrich | T7451 | 1:100 |
| Acef. Tubulin | | | |
| Rabbit anti- | AbCam | Ab193895 | 1:400 |
| Cytokeratin 5 | | | |
| Mouse anti- | Santa Cruz | Sc-365992 | 1:100 |
| SCGB1A1 | | | |
| Goat anti-ACE2 | AbCam | AF933 | 1.100 |
| Sheep anti- | Novus | AF4218 | 1:100 |
| SCGB1A1 | | | |
| Rabbit | Invitrogen | 617300 | 1:100 |
| anti-ZO-1 | | | |
| Mouse anti- | Sinobiological | 40143-MM05 | 1:100 |
| SARS-CoV2 NP | | | |
| Mouse anti- | Scicons | 10010200 | 1:100 |
| dsRNA | | | |
| Rabbit anti- | Cell Signaling | 5335 | 1:100 |
| acetyl-α- | | | |
| tubulin | | | |
| Blocking | Jackson | 017-000-121 | 1:10 |
| Serum | Immmunoresearch | 005-000-121 | |
| Secondary | Jackson | 711-165-152 | 1:400 |
| Antibodies | Immunoresearch | 711-095-152 | |
| | | 715-165-151 | |
| | | 715-095-151 | |
| Anti-mouse | Invitrogen | A-21042 | 1:400 |
| IgM | | | |
| Anti-mouse | Invitrogen | A-21422 | 1:400 |
| IgG | | | |
| Alexa Fluor | Thermo Fisher | A22285 | 1:100 |
| 660 Phalloidin | | | |
| Mouse anti- | Biolegend | Clone ITEM-4 | 1:100 |
| TNFRSF12A | | 314102 | |
| Rabbit anti- | Thermo Fisher | PA5-20275, lot | 1:100 |
| TNFRSF12A | | TD2561104 | |

qRT-PCR Reagents

| | Reagent | Vendor | Catalog # |
|---|---|---|---|
| RNA Isolation | PicoPure ™ RNA | Applied Biosysterns | KIT0214 |
| | Isolation Kit | | |
| cDNA Synthesis | iScript ™ cDNA | Bio-Rad | 1708841 |
| | Synthesis Kit | | |
| Pre-amplification | SsoAdynnced ™ | Bio-Rad | 1725160 |
| | PreAmp Supermix | | |
| Taqman Gene | GAPDH | Thermo Fisher | Hs02786624_g1 |
| Expression Assay | | | |
| | ITGA6 | Thermo Fisher | Hs01041011 m1 |
| | ITGB4 | Thermo Fisher | Hs00236216_m1 |
| | KRT5 | Thermo Fisher | Hs00361185_m1 |
| | TNFRSF12A | Thermo Fisher | Hs00171993_m1 |
| COV2 RT-PCR Assay | nCOV N1 FWD | IDT | 10008321 |
| | nCOV_N1 REV | IDT | 10006822 |
| | nCOV N1 Probe | IDT | 10006823 |
| | TaqMan ™ Universal | Applied Biosystems | 4440040 |
| | Master Mix II, no UNG | | |

qRT-PCR Primers

| | Sequence 5′→3′ |
|---|---|
| SARS unspliced FWD | TGACTTCACGGAAGAGAGGTT |
| SARS unspliced REV | AACAGTTAACACAATTTGGGTGG |
| Human U3 FWD | CGTGTAGAGCACCGAAAACC |
| Human U3 REV | AACAGTTAACACAATTTGGGTGG |

1. A method for culture of human distal lung organoid cultures, comprising
obtaining a human peripheral lung tissue sample;
dissociating the peripheral lung tissue into single cells;
culturing the cells in culture medium comprising extracellular matrix and an effective concentration of factors, for a period of time sufficient to form organoids.

2. The method of claim 1, wherein the organoids are everted by the process of:
removing organoids from extracellular matrix culture;
placing the organoids in suspension culture, thereby leading to relocation of differentiated cells from the lumen of the organoid, to the organoid exterior.

3. The method of claim 1, wherein the organoids are viable in culture for a period of at least 3 months.

4. The method of claim 1, wherein the organoids comprise one or more of human AT2 cells, KRT5+ basal cells, and club cells.

5. The method of claim 1, wherein the factors in the culture medium comprise epidermal growth factor (EGF) and a BMP antagonist.

6. The method of claim 5, wherein the BMP antagonist is NOGGIN protein.

7. The method of claim 5, wherein the medium further comprises an inhibitor of TGF-β.

8. The method of claim 1, wherein the peripheral lung tissue is lung parenchyma from the visceral pleura.

9. The method of claim 1, further comprising the step of isolating single cells from the organoid.

10. The method of claim 9, wherein the isolated single cells are cultured to an organoid.

11. The method of claim 1, further comprising infecting the organoid with a respiratory pathogen.

12. The method of claim 11, wherein the pathogen is influenza virus.

13. The method of claim 11, wherein the pathogen is SARS-CoV-2 and the organoid has been everted.

14. A method for screening a candidate agent for an effect on a mammalian tissue, the method comprising:
contacting a candidate agent with an organoid culture according to claim 1, and determining the effect of the agent on the organoids in the culture.

15. The method of claim 14, wherein the candidate agent is an anti-viral therapeutic agent, and the organoid has been infected with a respiratory virus.
